(19) United States
(12) Patent Application Publication
(10) Pub. No.: US 2023/0321146 A1
(43) Pub. Date: Oct. 12, 2023

(54) CHIMERIC POLYPEPTIDES AND METHODS OF USING THE SAME

(71) Applicant: REFUGE BIOTECHNOLOGIES, INC., Menlo Park, CA (US)

(72) Inventors: Zhifen Yang, Menlo Park, CA (US); Lingyu Li, Millbrae, CA (US)

(21) Appl. No.: 17/979,541

(22) Filed: Nov. 2, 2022

Related U.S. Application Data
(63) Continuation of application No. 17/708,536, filed on Mar. 30, 2022, now abandoned.
Application No. 17/708,536 is a continuation of application No. 17/407,600, filed on Aug. 20, 2021, now abandoned.
Application No. 17/407,600 is a continuation of application No. PCT/US2020/019358, filed on Feb. 21, 2020.
(60) Provisional application No. 62/867,120, filed on Jun. 26, 2019.
(60) Provisional application No. 62/809,477, filed on Feb. 22, 2019.

(51) Int. Cl. A61K 35/17; C07K 14/725; A61P 35/00

(52) U.S. Cl. CPC A61K 35/17; A61P 35/00; C07K 14/7051

(57) ABSTRACT

The present disclosure provides a chimeric polypeptide comprising at least one heterologous nuclear export signal linked to an adaptor protein of a receptor. The adaptor protein may be a Linker for Activation of T cell (LAT). The receptor may comprise a chimeric receptor.

### CROSS-REFERENCE

This application is a continuation of U.S. Patent Application No. 17/708,536, filed Mar. 30, 2022, which is a continuation of U.S. Pat. Application No. 17/407,600, filed Aug. 20, 2021, which is continuation of International Patent Application No. PCT/US20/19358, filed Feb. 21, 2020, which claims the benefit of U.S. Pat. Application No. 62/809,477, filed Feb. 22, 2019 and U.S. Pat. Application No. 62/867,120, filed Jun. 26, 2019, each of which is entirely incorporated herein by reference.

### SEQUENCE LISTING

[0001.1] The instant application contains a Sequence Listing which has been submitted electronically in XML format and is hereby incorporated by reference in its entirety. Said XML copy, created on Jun. 14, 2023, is named 50489-718_303_SL.xml and is 14,222 bytes in size.

### BACKGROUND

Regulation of cell activities can involve the binding of a ligand to a membrane-bound receptor comprising a ligand binding domain and a signaling domain. Formation of a complex between a ligand and the ligand binding domain can result in a conformational and/or chemical modification in the receptor which can result in a signal transduced within the cell. In some situations, a portion of the receptor of the signaling domain or adjacent to the signaling domain is phosphorylated (e.g., trans- and/or auto-phosphorylated), resulting in a change in its activity. These events can be coupled with secondary messengers and/or the recruitment of co-factor moieties (e.g., proteins). In some instances, the change in such portion of the receptor results in binding to other signaling moieties (e.g., adaptor proteins, co-factor proteins, and/or other receptors). These other signaling moieties can be activated and then carry out various functions within a cell.

In some examples, T cell receptor (TCR) utilizes Linker for Activation of T-cells (LAT) as one of the signaling moieties to be activated and to carry out various functions of the cell (e.g., an immune cell, such as a T cell).

### SUMMARY

In an aspect, the present disclosure provides a chimeric polypeptide comprising at least one heterologous nuclear export signal (NES) linked to an adaptor protein of a receptor.

In some embodiments, the receptor comprises a transmembrane receptor or nuclear membrane receptor. In some embodiments, the receptor comprises a transmembrane receptor and nuclear membrane receptor. In some embodiments, the receptor comprises a chimeric antigen receptor (CAR) or a T cell receptor (TCR). In some embodiments, the receptor comprises a chimeric antigen receptor (CAR) and a T cell receptor (TCR). In some embodiments, the at least one heterologous NES is linked to C-terminus or N-terminus of the adaptor protein. In some embodiments, the at least one heterologous NES is linked to C-terminus and N-terminus of the adaptor protein. In some embodiments, the adaptor protein is flanked by a first heterologous NES and a second heterologous NES.

In some embodiments, the adaptor protein comprises a fragment thereof and/or a functional variant thereof. In some embodiments, the adaptor protein is selected from the group consisting of: 14-3-3 beta, 14-3-3 gamma, 14-3-3 epsilon, 14-3-3 eta, 14-3-3 sigma, 14-3-3 theta, 14-3-3 zeta, ALX, APBA2, APBA3, APPL, BAP31, BCAP, BLNK, BRDG1, CARD9, CBL, CD2AP, CHIP/STUB1, CIDEA, CIDEC, CIN85/SH3KBP1, CRADD, Crk, CrkL, DAB2, DAP10/HCST, DAPP1, Daxx, DFF40/CAD, DFF45/ICAD, DISC1, DOCK1, DOCK2, DOCK3, DOK3, DOK7, Erbin, FADD, FLIP, FRS2, GAB2, GAB3, GAPDH, GAPDH-2, GBL, GRAP2, GRB2, GRB7, GULP1/CED-6, Importin alpha 2/KPNA2, Importin alpha 3/KPNA4, Importin alpha 5/KPNA1, Importin beta/KPNB1, IRS1, IRS2, a Linker for Activation of T cells (LAT), LNK/SH2B3, Mena, MIG2, MyD88, NCK1, NOD1, NTAL, Numb, p130Cas, p62/SQSTM1, PAG1, PARD3/Par3, Paxillin, PDHX, PINCH1, Ras-GAP, RIAM/APBB1IP, SAM68, SH2B1, SH2D1A, SH2D2A, SHANK2, SHB, SHC1, SIT1, SOCS-5, SOCS-6, SOCS-7/Nck/NAP4, STAM-1, STI1, SWAP70, TANK, TAPP1, Tollip, TRADD, TRAF-1, TRAF-2, TRAF-3, TRAF-4, TRAF-5, TRAF-6, TRAM/TICAM2, TRIF/TICAM1, TRRAP, UBASH3B/STS1, Vav-1, ARRB2, c-Cbl, GADS, Lck, SLP-76, ZAP-70, Sos1, plc-γ1, PI3K, DAG, a fragment thereof, a functional variant thereof, and a combination thereof.

In some embodiments, the adaptor protein comprises the LAT. In some embodiments, the LAT comprises at least one isoform of the LAT.

In some embodiments, upon introduction of the chimeric polypeptide into a cell comprising the receptor, the chimeric polypeptide prolongs or enhances signaling of the receptor in the cell, as compared to the adaptor protein without the at least one heterologous NES. In some embodiments, the at least one heterologous NES (i) enhances translocation of the adaptor protein into a membrane of the cell, (ii) reduces displacement of the adaptor protein from a membrane of the cell during the signaling of the receptor, (iii) reduces degradation of the adaptor protein during the signaling of the receptor, or (iv) stabilizes the adaptor protein in a membrane of the cell, as compared to the adaptor protein without the at least one heterologous NES. In some embodiments, the at least one heterologous NES enhances translocation of the adaptor protein into a membrane of the cell. In some embodiments, the at least one heterologous NES reduces displacement of the adaptor protein from a membrane of the cell during the signaling of the receptor.

In some embodiments, the at least one heterologous NES comprises a polynucleotide pattern comprising LxxLxL, LxxxLxL, or LxxxLxxLxL, wherein each L is a hydrophobic amino acid residue. In some embodiments, the at least one heterologous NES comprises a polynucleotide pattern comprising two or more of LxxLxL, LxxxLxL, and LxxxLxxLxL, wherein each L is a hydrophobic amino acid residue. In some embodiments, the hydrophobic amino acid residue is selected from the group consisting of leucine, isoleucine, valine, phenylalanine, and methionine. In some embodiments, a sequence of the at least one NES is LALKLAGLDI (SEQ ID NO: 2) or LQLPPLERLTL (SEQ ID NO:3). In some embodiments, a sequence of the at least one NES is LALKLAGLDI (SEQ ID NO: 2) and LQLPPLERLTL (SEQ ID NO:3).

In some embodiments, a portion of the chimeric polypeptide encoding the adaptor protein comprises at least one mutation, as compared to a wild-type adaptor protein of the cell or a different type of cell. In some embodiments, upon introduction of the chimeric polypeptide into a cell comprising the receptor, the at least one mutation (i) reduces displacement of the adaptor protein from a membrane of the cell during signaling of the receptor, or (ii) reduces ubiquitination of the adaptor protein during signaling of the receptor, as compared the wild-type adaptor protein without the at least one mutation. In some embodiments, upon introduction of the chimeric polypeptide into a cell comprising the receptor, the at least one mutation reduces displacement of the adaptor protein from a membrane of the cell during signaling of the receptor. In some embodiments, upon introduction of the chimeric polypeptide into a cell comprising the receptor, the at least one mutation reduces ubiquitination of the adaptor protein during signaling of the receptor, as compared the wild-type adaptor protein without the at least one mutation. In some embodiments, upon introduction of the chimeric polypeptide into a cell comprising the receptor, the at least one mutation (i) reduces displacement of the adaptor protein from a membrane of the cell during signaling of the receptor, and (ii) reduces ubiquitination of the adaptor protein during signaling of the receptor, as compared the wild-type adaptor protein without the at least one mutation.

In some embodiments, the at least one mutation is at one or more cysteine residues of the wild-type adaptor protein. In some embodiments, the at least one mutation comprises (i) deletion of the one or more cysteine residues, or (ii) substitution of the one or more cysteine residues with one or more different amino acid residues that are natural or synthetic. In some embodiments, the at least one mutation comprises (i) deletion of the one or more cysteine residues, and (ii) substitution of the one or more cysteine residues with one or more different amino acid residues that are natural or synthetic.

In some embodiments, the at least one mutation is at one or more lysine residues of the wild-type adaptor protein. In some embodiments, the at least mutation comprises (i) deletion of the one or more lysine residues, or (ii) substitution of the one or more lysine residues with one or more different amino acid residues that are natural or synthetic. In some embodiments, the at least mutation comprises (i) deletion of the one or more lysine residues, and (ii) substitution of the one or more lysine residues with one or more different amino acid residues that are natural or synthetic. In some embodiments, the wild-type adaptor protein is a human wild-type adaptor protein.

In some embodiments, the chimeric polypeptide further comprises at least one additional polypeptide, wherein upon introduction of the chimeric polypeptide into a cell, a charge, size, and/or position of the at least one additional polypeptide relative to the chimeric polypeptide is sufficient to inhibit or reduce interaction between the adaptor protein and a cellular component of the cell. In some embodiments, the at least one additional polypeptide reduces degradation of the adaptor protein in the cell, as compared to the adaptor protein without the at least one additional polypeptide. In some embodiments, the at least one additional polypeptide is disposed at an intracellular portion of the chimeric polypeptide. In some embodiments, the at least one additional polypeptide is disposed at or adjacent to (i) C-terminus or (ii) N-terminus of the chimeric polypeptide. In some embodiments, (i) the at least one additional polypeptide is flanked by the adaptor protein and the at least one heterologous NES, or (ii) the at least one heterologous NES is flanked by the adaptor protein and the at least one additional polypeptide. In some embodiments, the cellular component comprises a nucleic acid, a polynucleotide, an amino acid, a polypeptide, lipid, a carbohydrate, a small molecule, an enzyme, a ribosome, a proteasome, a variant thereof, or any combination thereof.

In some embodiments, the chimeric polypeptide further comprises a recognition moiety that is specifically recognized by an antibody. In some embodiments, upon introduction of the chimeric polypeptide into a cell, contacting of the recognition moiety by the antibody promotes or enhances (i) antibody-dependent cellular cytotoxicity, or (ii) complement-dependent cytotoxicity of the cell. In some embodiments, the recognition moiety comprises epidermal growth factor receptor (EGFR) or a fragment thereof. In an example, the fragment of EGFR may comprise at least one domain of the EGFR (e.g., at least 1, 2, 3, 4, 5, or more domains of the EGFR fused as a single polypeptide). In some embodiments, the antibody comprises at least one toxin capable of inducing death of the cell.

In another aspect, the present disclosure provides a polynucleotide encoding any one of the subject chimeric polypeptides. In another aspect, the present disclosure provides an expression cassette comprising the polynucleotide, wherein the polynucleotide is operatively linked to a regulatory sequence. In another aspect, the present disclosure provides a composition comprising at least the expression cassette, wherein the at least the expression cassette is in a pharmaceutically acceptable carrier. In another aspect, the present disclosure provides a composition comprising any one of the subject chimeric polypeptides, wherein any one of the subject chimeric polypeptides is in a pharmaceutically acceptable carrier. In another aspect, the present disclosure provides a kit comprising the composition. In another aspect, the present disclosure provides a cell comprising any one of the subject chimeric polypeptides. In some embodiments, the cell further comprises the receptor. In some embodiments, the cell is a host cell.

In another aspect, the present disclosure provides a system for regulating signaling of a receptor in a cell, comprising: a chimeric polypeptide comprising an adaptor protein of a receptor linked to at least one heterologous nuclear export signal (NES), wherein, upon introducing the system into the cell, the chimeric polypeptide prolongs or enhances the signaling of the receptor, as compared to the adaptor protein without the at least one heterologous NES.

In some embodiments, the at least one heterologous NES (i) enhances translocation of the adaptor protein into a membrane of the cell, (ii) reduces displacement of the adaptor protein from a membrane of the cell during the signaling of the receptor, (iii) reduces degradation of the adaptor protein during the signaling of the receptor, or (iv) stabilizes the adaptor protein in a membrane of the cell, as compared to the adaptor protein without the at least one heterologous NES. In some embodiments, the at least one heterologous NES enhances translocation of the adaptor protein into a membrane of the cell. In some embodiments, the at least one heterologous NES reduces displacement of the adaptor protein from a membrane of the cell during the signaling of the receptor. In some embodiments, the membrane of the cell comprises transmembrane and/or nuclear membrane of the cell. In some embodiments, he at least one heterologous NES comprises a polynucleotide pattern comprising LxxLxL, LxxxLxL, and/or LxxxLxxLxL, wherein each L is a hydrophobic amino acid residue. In some embodiments, the hydrophobic amino acid residue is selected from the group consisting of leucine, isoleucine, valine, phenylalanine, and methionine. In some embodiments, a sequence of the at least one NES is LALKLAGLDI (SEQ ID NO: 2) or LQLPPLERLTL (SEQ ID NO:3). In some embodiments, a sequence of the at least one NES is LALKLAGLDI (SEQ ID NO: 2) and LQLPPLERLTL (SEQ ID NO:3).

In some embodiments, the adaptor protein is selected from the group consisting of: 14-3-3 beta, 14-3-3 gamma, 14-3-3 epsilon, 14-3-3 eta, 14-3-3 sigma, 14-3-3 theta, 14-3-3 zeta, ALX, APBA2, APBA3, APPL, BAP31, BCAP, BLNK, BRDG1, CARD9, CBL, CD2AP, CHIP/STUB 1, CIDEA, CIDEC, CIN85/SH3KBP1, CRADD, Crk, CrkL, DAB2, DAP10/HCST, DAPP1, Daxx, DFF40/CAD, DFF45/ICAD, DISC1, DOCK1, DOCK2, DOCK3, DOK3, DOK7, Erbin, FADD, FLIP, FRS2, GAB2, GAB3, GAPDH, GAPDH-2, GBL, GRAP2, GRB2, GRB7, GULP1/CED-6, Importin alpha 2/KPNA2, Importin alpha 3/KPNA4, Importin alpha 5/KPNA1,Importin beta/KPNB1, IRS1, IRS2, a Linker for Activation of T cells (LAT), LNK/SH2B3, Mena, MIG2, MyD88, NCK1, NOD1, NTAL, Numb, p130Cas, p62/SQSTM1, PAG1, PARD3/Par3, Paxillin, PDHX, PINCH1, Ras-GAP, RIAM/APBB1IP, SAM68, SH2B1, SH2D1A, SH2D2A, SHANK2, SHB, SHC1, SIT1, SOCS-5, SOCS-6, SOCS-7/Nck/NAP4, STAM-1, STI1, SWAP70, TANK, TAPP1, Tollip, TRADD, TRAF-1, TRAF-2, TRAF-3, TRAF-4, TRAF-5, TRAF-6, TRAM/TICAM2, TRIF/TICAM1, TRRAP, UBASH3B/STS1, Vav-1, ARRB2, c-Cbl, GADS, Lck, SLP-76, ZAP-70, Sos1, plc-γ1, PI3K, DAG, a fragment thereof, a functional variant thereof, and a combination thereof. In some embodiments, the adaptor protein comprises the LAT. In some embodiments, the LAT comprises at least one isoform of the LAT.

In some embodiments, the at least one heterologous NES is linked to C-terminus and/or N-terminus of the adaptor protein. In some embodiments, the adaptor protein is flanked by a first heterologous NES and a second heterologous NES.

In some embodiments, the chimeric polypeptide prolongs and/or enhances proliferation of the cell, as compared to the adaptor protein without the at least one heterologous NES.

In some embodiments, the adaptor protein comprises at least one mutation as compared to a wild-type adaptor protein, wherein the at least one mutation (i) reduces displacement of the adaptor protein from a membrane of the cell during the signaling of the receptor, or (ii) reduces ubiquitination of the adaptor protein during the signaling of the receptor, as compared the wild-type adaptor protein without the at least one mutation. In some embodiments, the adaptor protein comprises at least one mutation as compared to a wild-type adaptor protein, wherein the at least one mutation reduces displacement of the adaptor protein from a membrane of the cell during the signaling of the receptor. In some embodiments, the adaptor protein comprises at least one mutation as compared to a wild-type adaptor protein, wherein the at least one mutation reduces ubiquitination of the adaptor protein during the signaling of the receptor, as compared the wild-type adaptor protein without the at least one mutation. In some embodiments, the adaptor protein comprises at least one mutation as compared to a wild-type adaptor protein, wherein the at least one mutation (i) reduces displacement of the adaptor protein from a membrane of the cell during the signaling of the receptor, and (ii) reduces ubiquitination of the adaptor protein during the signaling of the receptor, as compared the wild-type adaptor protein without the at least one mutation.

In some embodiments, the at least one mutation is at one or more cysteine residues of the wild-type adaptor protein. In some embodiments, the at least mutation comprises (i) deletion of the one or more cysteine residues, or (ii) substitution of the one or more cysteine residues with one or more different amino acid residues that are natural or synthetic. In some embodiments, the at least mutation comprises (i) deletion of the one or more cysteine residues, and (ii) substitution of the one or more cysteine residues with one or more different amino acid residues that are natural or synthetic.

In some embodiments, the at least one mutation is at one or more lysine residues of the wild-type adaptor protein. In some embodiments, the at least mutation comprises (i) deletion of the one or more lysine residues, or (ii) substitution of the one or more lysine residues with one or more different amino acid residues that are natural or synthetic. In some embodiments, the at least mutation comprises (i) deletion of the one or more lysine residues, and (ii) substitution of the one or more lysine residues with one or more different amino acid residues that are natural or synthetic.

In some embodiments, the wild-type adaptor protein is a human wild-type adaptor protein.

In some embodiments, the chimeric polypeptide further comprises at least one additional polypeptide, wherein a charge, size, or position of the at least one additional polypeptide relative to the chimeric polypeptide is sufficient to inhibit or reduce interaction between the adaptor protein and a cellular component. In some embodiments, the at least one additional polypeptide reduces degradation of the adaptor protein in the cell, as compared to the adaptor protein without the at least one additional polypeptide. In some embodiments, the at least one additional polypeptide is disposed at an intracellular portion of the chimeric polypeptide. In some embodiments, the at least one additional polypeptide is disposed at or adjacent to C-terminus of the chimeric polypeptide. In some embodiments, (i) the at least one additional polypeptide is flanked by the adaptor protein and the at least one heterologous NES, or (ii) the at least one heterologous NES is flanked by the adaptor protein and the at least one additional polypeptide. In some embodiments, the at least one additional polypeptide is flanked by the adaptor protein and the at least one heterologous NES. In some embodiments, the at least one heterologous NES is flanked by the adaptor protein and the at least one additional polypeptide. In some embodiments, the cellular component comprises a nucleic acid, a polynucleotide, an amino acid, a polypeptide, lipid, a carbohydrate, a small molecule, an enzyme, a ribosome, a proteasome, a variant thereof, or any combination thereof.

In some embodiments, the chimeric polypeptide further comprises a recognition moiety that is specifically recognized by an antibody. In some embodiments, contacting of the recognition moiety by the antibody promotes or enhances (i) antibody-dependent cellular cytotoxicity, or (ii) complement-dependent cytotoxicity of the cell. In some embodiments, the recognition moiety comprises epidermal growth factor receptor (EGFR) or a fragment thereof. In some embodiments, the antibody comprises at least one toxin capable of inducing death of the cell.

In some embodiments, the receptor comprises a ligand binding domain specific for a ligand, and wherein the receptor is activated upon binding of the ligand to the ligand binding domain. In some embodiments, the ligand is an extracellular ligand. In some embodiments, the extracellular ligand is an antigen presented on a target cell of the cell. In some embodiments, the antigen is membrane bound or non-membrane bound. In some embodiments, the chimeric polypeptide prolongs or enhances cytotoxicity of the cell against the target cell, as compared to the adaptor moiety without the at least one heterologous NES. In some embodiments, the target cell comprises a tumor cell or a cancer cell. In some embodiments, the chimeric polypeptide reduces a size of or obliterates a tumor, as compared to the adaptor moiety without the at least one heterologous NES.

In some embodiments, the receptor is heterologous to the cell. In some embodiments, the heterologous receptor comprises a chimeric antigen receptor (CAR). In some embodiments, the CAR comprises at least a portion of a Notch receptor, a G-protein coupled receptor (GPCR), an integrin receptor, a cadherin receptor, a receptor tyrosine kinase, a death receptor, or an immune receptor. In some embodiments, the immune receptor comprises a T cell receptor (TCR). In some embodiments, the receptor is endogenous to the cell. In some embodiments, the endogenous receptor comprises a Notch receptor, a G-protein coupled receptor (GPCR), an integrin receptor, a cadherin receptor, a receptor tyrosine kinase, a death receptor, or an immune receptor. In some embodiments, the immune receptor comprises a T cell receptor (TCR).

In some embodiments, the system further comprises (i) a gene modulating polypeptide (GMP) comprising an actuator moiety linked to a cleavage recognition site, wherein the actuator moiety regulates the expression of the target polynucleotide in the cell, and (ii) a cleavage moiety capable of cleaving the cleavage recognition site of the GMP, wherein activation of the receptor induces the cleavage moiety to cleave the cleavage recognition site, to effect regulating expression of the target polynucleotide in the cell. In some embodiments, the receptor comprises the GMP and the chimeric polypeptide comprises the cleavage moiety. In some embodiments, the chimeric polypeptide comprises the GMP and the receptor comprises the cleavage moiety. In some embodiments, the system further comprises an additional polypeptide that comprises (i) the GMP and (ii) a coupling polypeptide configured to bind the receptor or a downstream signaling moiety of the receptor in response to the activation of the receptor. In some embodiments, the receptor comprises the cleavage moiety. In some embodiments, the chimeric polypeptide comprises the cleavage moiety. In some embodiments, the system further comprises an additional polypeptide that comprises (i) the cleavage moiety and (ii) a coupling polypeptide configured to bind the receptor or a downstream signaling moiety of the receptor in response to the activation of the receptor. In some embodiments, the receptor comprises the GMP. In some embodiments, the chimeric polypeptide comprises the GMP.

In another aspect, the present disclosure provides a polynucleotide encoding any one of the subject systems. In another aspect, the present disclosure provides an expression cassette comprising the polynucleotide, wherein the polynucleotide is operatively linked to a regulatory sequence. In some embodiments, the regulatory sequence is endogenous or exogenous to the cell. In another aspect, the present disclosure provides a composition comprising one or more polynucleotides that encode any one of the subject systems. In some embodiments, the one or more polynucleotides are in a pharmaceutically acceptable carrier. In another aspect, the present disclosure provides a kit comprising the composition.

In another aspect, the present disclosure provides an isolated host cell expressing any one of the subject systems. In some embodiments, the host cell is an immune cell. In some embodiments, the immune cell is a lymphocyte. In some embodiments, the lymphocyte is a T cell. In some embodiments, the T cell is selected from the group consisting of: Cytotoxic T cell, Natural Killer T cell, Regulatory T cell, and T helper cell. In some embodiments, the host cell is a hematopoietic stem cell or an Induced pluripotent stem cell (iPSC).

In another aspect, the present disclosure provides a method of enhancing signaling of a receptor in a cell, comprising: expressing a system in the cell, wherein the system comprises a chimeric polypeptide comprising an adaptor protein of a receptor linked to at least one heterologous nuclear export signal (NES), wherein the chimeric polypeptide enhances the signaling of the receptor, as compared to the adaptor protein without the at least one heterologous NES, and wherein the enhanced signaling of the receptor is evidenced by (i) enhanced viability of the cell, (ii) enhanced proliferation of the cell, (iii) enhanced intracellular signaling of the cell, (iv) enhanced cytotoxicity against a target cell, and/or (v) enhanced ability to reduce a size of or obliterate a tumor. In some embodiments, the enhanced signaling is evidenced by enhanced viability of the cell. In some embodiments, the enhanced signaling is evidenced by enhanced proliferation of the cell. In some embodiments, the enhanced signaling is evidenced by enhanced intracellular signaling of the cell. In some embodiments, the enhanced signaling is evidenced by enhanced cytotoxicity against a target cell. In some embodiments, the target cell is a tumor cell or a cancer cell. In some embodiments, the enhanced signaling is evidenced by enhanced ability to reduce a size of or obliterate a tumor. In some embodiments, the enhanced signaling of the receptor is measured during and/or subsequent to activation of the receptor.

In another aspect, the present disclosure provides a method of increasing half-life of an adaptor protein of a receptor in a cell, comprising: expressing a system in the cell, wherein the system comprises a chimeric polypeptide comprising the adaptor protein of the receptor linked to at least one heterologous nuclear export signal (NES), wherein the increase in the half-life of the adaptor protein linked to the at least one heterologous NES, as compared to the adaptor protein without the at least one heterologous NES, is evidenced by (i) increased amount of the adaptor protein that is membrane bound in the cell and/or (ii) higher steady state amount of the adaptor protein in the cell. In some embodiments, the increase in the half-life of the adaptor protein linked to the at least one heterologous NES is evidenced by the increased amount of the adaptor protein that is membrane bound in the cell. In some embodiments, the increase in the half-life of the adaptor protein linked to the at least one heterologous NES is evidenced by the higher steady state amount of the adaptor protein in the cell. In some embodiments, the half-life of the adaptor protein is measured prior to, during, and/or subsequent to activation of the receptor.

In some embodiments, the at least one heterologous NES (i) enhances translocation of the adaptor protein into a membrane of the cell, (ii) reduces displacement of the adaptor protein from a membrane of the cell during the signaling of the receptor, (iii) reduces degradation of the adaptor protein during the signaling of the receptor, or (iv) stabilizes the adaptor protein in a membrane of the cell, as compared to the adaptor protein without the at least one heterologous NES. In some embodiments, the at least one heterologous NES enhances translocation of the adaptor protein into a membrane of the cell. In some embodiments, the at least one heterologous NES reduces displacement of the adaptor protein from a membrane of the cell during the signaling of the receptor. In some embodiments, the membrane of the cell comprises transmembrane and/or nuclear membrane of the cell.

In some embodiments, the at least one heterologous NES comprises a polynucleotide pattern comprising LxxLxL, LxxxLxL, and/or LxxxLxxLxL, wherein each L is a hydrophobic amino acid residue. In some embodiments, the hydrophobic amino acid residue is selected from the group consisting of leucine, isoleucine, valine, phenylalanine, and methionine. In some embodiments, a sequence of the at least one NES is LALKLAGLDI (SEQ ID NO: 2) or LQLPPLERLTL (SEQ ID NO:3). In some embodiments, a sequence of the at least one NES is LALKLAGLDI (SEQ ID NO: 2) and LQLPPLERLTL (SEQ ID NO:3).

In some embodiments, the adaptor protein is selected from the group consisting of: 14-3-3 beta, 14-3-3 gamma, 14-3-3 epsilon, 14-3-3 eta, 14-3-3 sigma, 14-3-3 theta, 14-3-3 zeta, ALX, APBA2, APBA3, APPL, BAP31, BCAP, BLNK, BRDG1, CARD9, CBL, CD2AP, CHIP/STUB 1, CIDEA, CIDEC, CIN85/SH3KBP1, CRADD, Crk, CrkL, DAB2, DAP10/HCST, DAPP1, Daxx, DFF40/CAD, DFF45/ICAD, DISC1, DOCK1, DOCK2, DOCK3, DOK3, DOK7, Erbin, FADD, FLIP, FRS2, GAB2, GAB3, GAPDH, GAPDH-2, GBL, GRAP2, GRB2, GRB7, GULP1/CED-6, Importin alpha 2/KPNA2, Importin alpha 3/KPNA4, Importin alpha 5/KPNA1, Importin beta/KPNB1, IRS1, IRS2, a Linker for Activation of T cells (LAT), LNK/SH2B3, Mena, MIG2, MyD88, NCK1, NOD1, NTAL, Numb, p130Cas, p62/SQSTM1, PAG1, PARD3/Par3, Paxillin, PDHX, PINCH1, Ras-GAP, RIAM/APBB1IP, SAM68, SH2B1, SH2D1A, SH2D2A, SHANK2, SHB, SHC1, SIT1, SOCS-5, SOCS-6, SOCS-7/Nck/NAP4, STAM-1, STI1, SWAP70, TANK, TAPP1, Tollip, TRADD, TRAF-1, TRAF-2, TRAF-3, TRAF-4, TRAF-5, TRAF-6, TRAM/TICAM2, TRIF/TICAM1, TRRAP, UBASH3B/STS1, Vav-1, ARRB2, c-Cbl, GADS, Lck, SLP-76, ZAP-70, Sos1, plc-γ1, PI3K, DAG, a fragment thereof, a functional variant thereof, and a combination thereof. In some embodiments, the adaptor protein comprises the LAT. In some embodiments, the LAT comprises at least one isoform of the LAT.

In some embodiments, the at least one heterologous NES is linked to C-terminus and/or N-terminus of the adaptor protein. In some embodiments, the adaptor protein is flanked by a first heterologous NES and a second heterologous NES.

In some embodiments, the adaptor protein comprises at least one mutation as compared to a wild-type adaptor protein, wherein the at least one mutation (i) reduces displacement of the adaptor protein from a membrane of the cell during the signaling of the receptor, or (ii) reduces ubiquitination of the adaptor protein during the signaling of the receptor, as compared the wild-type adaptor protein without the at least one mutation.

In some embodiments, the at least one mutation is at one or more cysteine residues of the wild-type adaptor protein. In some embodiments, the at least mutation comprises (i) deletion of the one or more cysteine residues, or (ii) substitution of the one or more cysteine residues with one or more different amino acid residues that are natural or synthetic. In some embodiments, the at least mutation comprises (i) deletion of the one or more cysteine residues, and (ii) substitution of the one or more cysteine residues with one or more different amino acid residues that are natural or synthetic.

In some embodiments, the at least one mutation is at one or more lysine residues of the wild-type adaptor protein. In some embodiments, the at least mutation comprises (i) deletion of the one or more lysine residues, or (ii) substitution of the one or more lysine residues with one or more different amino acid residues that are natural or synthetic. In some embodiments, the at least mutation comprises (i) deletion of the one or more lysine residues, and (ii) substitution of the one or more lysine residues with one or more different amino acid residues that are natural or synthetic.

In some embodiments, the wild-type adaptor protein is a human wild-type adaptor protein.

In some embodiments, the chimeric polypeptide further comprises at least one additional polypeptide, wherein a charge, size, or position of the at least one additional polypeptide relative to the chimeric polypeptide is sufficient to inhibit or reduce interaction between the adaptor protein and a cellular component. In some embodiments, the at least one additional polypeptide reduces degradation of the adaptor protein in the cell, as compared to the adaptor protein without the at least one additional polypeptide. In some embodiments, the at least one additional polypeptide is disposed at an intracellular portion of the chimeric polypeptide. In some embodiments, the at least one additional polypeptide is disposed at or adjacent to C-terminus of the chimeric polypeptide. In some embodiments, (i) the at least one additional polypeptide is flanked by the adaptor protein and the at least one heterologous NES, or (ii) the at least one heterologous NES is flanked by the adaptor protein and the at least one additional polypeptide. In some embodiments, the cellular component comprises a nucleic acid, a polynucleotide, an amino acid, a polypeptide, lipid, a carbohydrate, a small molecule, an enzyme, a ribosome, a proteasome, a variant thereof, or any combination thereof.

In some embodiments, the chimeric polypeptide further comprises a recognition moiety that is specifically recognized by an antibody. In some embodiments, contacting of the recognition moiety by the antibody promotes or enhances (i) antibody-dependent cellular cytotoxicity, or (ii) complement-dependent cytotoxicity of the cell. In some embodiments, the recognition moiety comprises epidermal growth factor receptor (EGFR) or a fragment thereof. In some embodiments, the antibody comprises at least one toxin capable of inducing death of the cell.

In some embodiments, the receptor comprises a ligand binding domain specific for a ligand, and wherein the receptor is activated upon binding of the ligand to the ligand binding domain. In some embodiments, the ligand is an extracellular ligand. In some embodiments, the extracellular ligand is an antigen presented on a target cell of the cell. In some embodiments, the antigen is membrane bound or non-membrane bound.

In some embodiments, the receptor is heterologous to the cell. In some embodiments, the heterologous receptor comprises a chimeric antigen receptor (CAR). In some embodiments, the CAR comprises at least a portion of a Notch receptor, a G-protein coupled receptor (GPCR), an integrin receptor, a cadherin receptor, a receptor tyrosine kinase, a death receptor, or an immune receptor. In some embodiments, the immune receptor comprises a T cell receptor (TCR). In some embodiments, the receptor is endogenous to the cell. In some embodiments, the endogenous receptor comprises a Notch receptor, a G-protein coupled receptor (GPCR), an integrin receptor, a cadherin receptor, a receptor tyrosine kinase, a death receptor, or an immune receptor. In some embodiments, the immune receptor comprises a T cell receptor (TCR).

In some embodiments, the system further comprises (i) a gene modulating polypeptide (GMP) comprising an actuator moiety linked to a cleavage recognition site, wherein the actuator moiety regulates the expression of the target polynucleotide in the cell, and (ii) a cleavage moiety capable of cleaving the cleavage recognition site of the GMP, wherein activation of the receptor induces the cleavage moiety to cleave the cleavage recognition site, to effect regulating expression of the target polynucleotide in the cell. In some embodiments, the receptor comprises the GMP and the chimeric polypeptide comprises the cleavage moiety. In some embodiments, the chimeric polypeptide comprises the GMP and the receptor comprises the cleavage moiety. In some embodiments, the system further comprises an additional polypeptide that comprises (i) the GMP and (ii) a coupling polypeptide configured to bind the receptor or a downstream signaling moiety of the receptor in response to the activation of the receptor. In some embodiments, the receptor comprises the cleavage moiety. In some embodiments, the chimeric polypeptide comprises the cleavage moiety. In some embodiments, the system further comprises an additional polypeptide that comprises (i) the cleavage moiety and (ii) a coupling polypeptide configured to bind the receptor or a downstream signaling moiety of the receptor in response to the activation of the receptor. In some embodiments, the receptor comprises the GMP. In some embodiments, the chimeric polypeptide comprises the GMP.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A schematically illustrates three example systems comprising a receptor (e.g., a chimeric receptor) and an adaptor protein of the receptor. The adaptor protein may be a wild-type adaptor protein. Alternatively, the adaptor protein may be a chimeric adaptor protein comprising at least one nuclear localization signal (NES) and/or a gene modulating polypeptide (GMP) comprising an actuator (e.g., dCase9-KRAB) capable of modulating expression of a target polynucleotide (e.g., gene) in a cell;

FIG. 1B schematically illustrates effect of cell proliferation by three example systems comprising a receptor (e.g., a chimeric receptor) and an adaptor protein of the receptor. A primary human T cell may be used as a host cell to express the three example systems. The primary human T cell may be obtained from a plurality of donors. Proliferation of the host cell may be measured by expression of the receptor and/or one or more markers of the host cell (e.g., CD4 and/or CD8);

FIG. 2 schematically illustrates effect on cell cytotoxicity against target cells (e.g., tumor cells, such as ovarian tumor cells) by three example systems comprising a receptor (e.g., a chimeric receptor) and an adaptor protein of the receptor. A primary human T cell may be used as a host cell to express the three example systems. The primary human T cell may be obtained from a plurality of donors. The effect of cell cytotoxicity against target cells may be obtained by varying the ratio of the effector (e.g., T cells expressing one of the three example systems) to the target (e.g., the target cells) (i.e., E:T ratio);

FIG. 3 schematically illustrates effect on cell viability and/or recovery by three example systems comprising a receptor (e.g., a chimeric receptor) and an adaptor protein of the receptor. A primary human T cell may be used as a host cell to express the three example systems. The primary human T cell may be obtained from a plurality of donors. The effect on host cell viability and/or recovery may be obtained subsequent to performing a predetermined assay (e.g., killing of target cells) using the host cell;

FIG. 4A schematically illustrates vectors encoding a receptor (e.g., a chimeric receptor); and FIG. 4B schematically illustrates effect on receptor expression and/or stability by three example systems comprising a receptor (e.g., the chimeric receptor) and an adaptor protein of the receptor. A primary human T cell may be used as a host cell to express the three example systems. The primary human T cell may be obtained from a plurality of donors. The effect on receptor expression and/or stability may be obtained by antibody staining;

FIGS. 5A and 5B schematically illustrate the effect on ubiquitination and/or proteasome-mediated degradation of an adaptor protein of a receptor in a cell by the absence (FIG. 5A) or presence (FIG. 5B) of at least one heterologous nuclear export signal (NES) linked to the adaptor protein;

FIG. 6 schematically illustrates modifications of an adaptor protein of a receptor, and the effect of the modifications on ubiquitination and/or proteasome-mediated degradation of the adaptor protein;

FIG. 7 schematically illustrates another modification of an adaptor protein of a receptor, and the effect of such modification on ubiquitination and/or proteasome-mediated degradation of the adaptor protein;

FIG. 8 schematically illustrates various modifications of an adaptor protein of a receptor that may reduce or prevent ubiquitination of the adaptor protein;

FIGS. 9A-C schematically illustrates effect on anti-tumor activity of T cell by the presence of modified adaptor proteins; and

FIG. 10A schematically illustrates a vector encoding a receptor (e.g., a chimeric receptor) and a modified adaptor protein of the receptor (e.g., tEGFR/LAT), wherein the receptor and the modified adaptor protein are linked by a self-cleavage polypeptide; and FIG. 10B schematically illustrates variations of the modified adaptor protein of the receptor.

FIG. 11 illustrates in vitro tumor cell killing assays, where the system comprising a receptor and adaptor protein of the receptor and complexed with PD1 single guide RNA (PD1sg) shows increased T cell proliferation (bottom left panel) and cytokine productions (right panels).

FIG. 12A schematically illustrates the in vivo tumor cell killing assays, where 0.5 million (0.5M) tumor cells (FaDu-PDL1) can be sub-cutaneous (s.c.) implanted into a NOD scid gamma (NSG) mouse and the NSG mouse can subsequently treated by injecting 1 million (1 M) or 3 million (M) T cells transduced with the systems comprising a receptor (e.g., a chimeric receptor) and an adaptor protein of the receptor as described herein.

FIG. 12B illustrates cell surface markers and cell population of the T cells of FIG. 12A prior to the T cells being activated

FIG. 12C illustrates tumor growth curves of FIG. 12A.

FIG. 12D illustrates tumor growth spider plots of FIG. 12A.

FIG. 12E illustrates Kaplan-Meier survival curves of the NSG mice of FIG. 12A.

FIG. 12F illustrates flow cytometry analysis of the tumor samples isolated from the NSG mice of FIG. 12A.

FIG. 13A illustrates in vivo tumor cell killing effects in mice, where the tumors are derived from cell lines of ovarian cancer cells, SKOV3, and the mice are treated with T cells transduced with the systems comprising a receptor (e.g., a chimeric receptor) and an adaptor protein of the receptor as described herein.

FIG. 13B illustrates a Kaplan-Meier survival curve of the treated mice of FIG. 13A.

FIG. 14 illustrates additional modifications based on the systems comprising a receptor (e.g., a chimeric receptor) and an adaptor protein of the receptor as described herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M.J. MacPherson, B.D. Hames and G.R. Taylor eds. (1995)), Harlow and Lane, eds. (1988) Antibodies, A Laboratory Manual, and Culture of Animal Cells: A Manual of Basic Technique and Specialized Applications, 6th Edition (R.I. Freshney, ed. (2010)).

### Background

As used in the specification and claims, the singular forms “a,” “an,” and “the” include plural references unless the context clearly dictates otherwise. For example, the term “a transmembrane receptor” can include a plurality of transmembrane receptors.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The terms “cell death” or “death of a cell,” as used interchangeably herein, generally refer to a process or event that causes a cell to cease and/or diminish normal metabolism in vivo or in vitro. Cell death can be induced by the cell itself (self-induced) or by another cell (e.g., another cell of the same type or a different type). In some cases, cell death can include, but are not limited to, programmed cell death (i.e., apoptosis), gradual death of the cells as occurs in diseased states (i.e., necrosis), and more immediate cell death such as toxicity (e.g., cytotoxicity, such as acute cytotoxicity). In some cases, cell apoptosis can be extrinsic (e.g., via signaling through a cell surface receptor, such as a death receptor) or intrinsic (e.g., via mitochondrial pathway).

The term “receptor,” as used herein, generally refers to a molecule (e.g., a polypeptide) that has an affinity for a given ligand. Receptors can be naturally occurring or synthetic molecules. The given ligand (or ligand) can be naturally occurring or synthetic molecules. Receptors can be employed in an unaltered state or as aggregates with other species (e.g., with one or more co-receptors, one or more adaptors, lipid rafts, etc.). Examples of receptors may include, but are not limited to, cell membrane receptors, soluble receptors, cloned receptors, recombinant receptors, complex carbohydrates and glycoproteins hormone receptors, drug receptors, transmitter receptors, autocoid receptors, cytokine receptors, antibodies, antibody fragments, engineered antibodies, antibody mimics, molecular recognition units, adhesion molecules, agglutinins, integrins, selectins, nucleic acids and synthetic heteropolymers comprising amino acids, nucleotides, carbohydrates or nonbiologic monomers, including analogs and derivatives thereof, and conjugates or complexes formed by attaching or binding any of these molecules to a second molecule.

The terms “adaptor protein” or “adaptor polypeptide,” as used interchangeably herein, generally refers to a protein or polypeptide that can regulate signal transduction pathway of a receptor of a cell. The adaptor protein may govern cross-talk between the receptor and one or more intracellular signaling moieties (e.g., proteins, enzymes, etc.). The adaptor protein may directly bind the receptor. The adaptor protein may not directly bind the receptor, but may be recruited towards the receptor upon activation of the receptor. The adaptor protein may contain one or more recognition motifs, which may facilitate interactions between two or more proteins (e.g., between the receptor and a downstream effector molecule, between two or more downstream effector molecules of the receptor signal transduction pathway, etc.) involved in the signal transduction pathway of the receptor. In some cases, the adaptor protein may participate in the regulation of a diverse range of cellular and biological process, including, for example, cell survival, cell proliferation, cell differentiation, cell transdifferentiation, cell dedifferentiation, cell death, death of a target cell, and stress responses. The adaptor protein may be a transmembrane protein. Alternatively, the adaptor protein may not be transmembrane protein.

The term “cell membrane,” as used herein, generally refers to the boundary membrane, external membrane, interfacial membrane, protoplasmic membrane, or cell wall that separates the protoplasm of the cell from the outside. Thus, the term “cell membrane receptor” or “transmembrane receptor,” as used here, refers to a receptor in the boundary membrane, external membrane, interfacial membrane, protoplasmic membrane, or cell wall that separates the protoplasm of the cell from the outside.

The term “antigen,” as used herein, generally refers to a molecule or a fragment thereof (e.g., ligand) capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen.

The term “antibody,” as used herein, generally refers to a proteinaceous binding molecule with immunoglobulin-like functions. The term antibody includes antibodies (e.g., monoclonal and polyclonal antibodies), as well as variants thereof. Antibodies include, but are not limited to, immunoglobulins (Ig’s) of different classes (i.e. IgA, IgG, IgM, IgD and IgE) and subclasses (such as IgG1, IgG2, etc.). A variant can refer to a functional derivative or fragment which retains the binding specificity (e.g., complete and/or partial) of the corresponding antibody. Antigen-binding fragments include Fab, Fab′, F(ab′)2, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The terms “Fc receptor” or “FcR,” as used herein, generally refers to a receptor, or any variant thereof, that can bind to the Fc region of an antibody. In certain embodiments, the FcR is one which binds an IgG antibody (a gamma receptor, Fcgamma R) and includes receptors of the Fcgamma RI (CD64), Fcgamma RII (CD32), and Fcgamma RIII (CD16) subclasses, including allelic variants and alternatively spliced forms of these receptors. Fcgamma RII receptors include Fcgamma RIIA (an “activating receptor”) and Fcgamma RIIB (an “inhibiting receptor”), which have similar amino acid sequences that differ primarily in the cytoplasmic domains thereof. The term “FcR” also includes the neonatal receptor, FcRn, which is responsible for the transfer of maternal IgGs to the fetus.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism but which is introduced into the host organism by gene transfer (e.g., transgene). A non-native gene can also refer to a naturally occurring nucleic acid or polypeptide sequence that comprises mutations, insertions and/or deletions (e.g., non-native sequence).

The terms “target polynucleotide” and “target nucleic acid,” as used herein, generally refer to a nucleic acid or polynucleotide which is targeted by an actuator moiety of the present disclosure. A target polynucleotide can be DNA (e.g., endogenous or exogenous). DNA can refer to template to generate mRNA transcripts and/or the various regulatory regions which regulate transcription of mRNA from a DNA template. A target polynucleotide can be a portion of a larger polynucleotide, for example a chromosome or a region of a chromosome. A target polynucleotide can refer to an extrachromosomal sequence (e.g., an episomal sequence, a minicircle sequence, a mitochondrial sequence, a chloroplast sequence, etc.) or a region of an extrachromosomal sequence. A target polynucleotide can be RNA. RNA can be, for example, mRNA which can serve as template encoding for proteins. A target polynucleotide comprising RNA can include the various regulatory regions which regulate translation of protein from an mRNA template. A target polynucleotide can encode for a gene product (e.g., DNA encoding for an RNA transcript or RNA encoding for a protein product) or comprise a regulatory sequence which regulates expression of a gene product. In general, the term “target sequence” refers to a nucleic acid sequence on a single strand of a target nucleic acid. The target sequence can be a portion of a gene, a regulatory sequence, genomic DNA, cell free nucleic acid including cfDNA and/or cfRNA, cDNA, a fusion gene, and RNA including mRNA, miRNA, rRNA, and others. A target polynucleotide, when targeted by an actuator moiety, can result in altered gene expression and/or activity. A target polynucleotide, when targeted by an actuator moiety, can result in an edited nucleic acid sequence. A target nucleic acid can comprise a nucleic acid sequence that may not be related to any other sequence in a nucleic acid sample by a single nucleotide substitution. A target nucleic acid can comprise a nucleic acid sequence that may not be related to any other sequence in a nucleic acid sample by a 2, 3, 4, 5, 6, 7, 8, 9, or 10 nucleotide substitutions. In some embodiments, the substitution may not occur within 5, 10, 15, 20, 25, 30, or 35 nucleotides of the 5′ end of a target nucleic acid. In some embodiments, the substitution may not occur within 5, 10, 15, 20, 25, 30, 35 nucleotides of the 3′ end of a target nucleic acid.

The term “mutation,” as used herein, generally refers to a substitution of a residue within a sequence, e.g., a nucleic acid or amino acid sequence, with another residue, or a deletion or insertion of one or more residues within a sequence. One or more mutations may be described by identifying the original residue followed by the position of the residue within the sequence and by the identity of the newly substituted residue.

The terms “transfection” or “transfected” generally refers to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The term “vector,” as used herein, generally refers to a nucleic acid molecule capable transferring or transporting a payload nucleic acid molecule. The payload nucleic acid molecule can be generally linked to, e.g., inserted into, the vector nucleic acid molecule. A vector may include sequences that direct autonomous replication in a cell, or may include sequences sufficient to allow integration into host cell gene (e.g., host cell DNA). Examples of a vector may include, but are not limited to, plasmids (e.g., DNA plasmids or RNA plasmids), transposons, cosmids, bacterial artificial chromosomes, and viral vectors.

A “plasmid,” as used herein, generally refers to a non-viral expression vector, e.g., a nucleic acid molecule that encodes for genes and/or regulatory elements necessary for the expression of genes. A “viral vector,” as used herein, generally refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to Gamma-retroviral, Alpha-retroviral, Foamy viral, lentiviral, adenoviral, or adeno-associated viral vectors.

A vector of any of the embodiments of the present disclosure can comprise exogenous, endogenous, or heterologous control sequences such as promoters and/or enhancers. An “endogenous” control sequence is one which is naturally linked to a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular gene therapy.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, AT, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

Complementarity can be perfect or substantial/sufficient. Perfect complementarity between two nucleic acids can mean that the two nucleic acids can form a duplex in which every base in the duplex is bonded to a complementary base by Watson-Crick pairing. Substantial or sufficient complementary can mean that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods.

The term “regulating” with reference to expression or activity, as used herein, generally refers to altering the level of expression or activity. Regulation can occur at the transcriptional level, post-transcriptional level, translational level, and/or post-translational level.

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “variant,” when used herein with reference to a polypeptide, generally refers to a polypeptide related, but not identical, to a wild type polypeptide, for example either by amino acid sequence, structure (e.g., secondary and/or tertiary), activity (e.g., enzymatic activity) and/or function. Variants include polypeptides comprising one or more amino acid variations (e.g., mutations, insertions, and deletions), truncations, modifications, or combinations thereof compared to a wild type polypeptide. Variants also include derivatives of the wild type polypeptide and fragments of the wild type polypeptide.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “gene modulating polypeptide” or “GMP,” as used herein, generally refers to a polypeptide comprising at least an actuator moiety capable of regulating expression or activity of a gene and/or editing a nucleic acid sequence. A GMP can comprise additional peptide sequences which are not directly involved in modulating gene expression, for example targeting sequences, polypeptide folding domains, etc.

The term “actuator moiety,” as used herein, generally refers to a moiety which can regulate expression or activity of a gene and/or edit a nucleic acid sequence, whether exogenous or endogenous. An actuator moiety can regulate expression of a gene at the transcriptional level, post-transcriptional level, translational level, and/or post-translation level. An actuator moiety can regulate gene expression at the transcription level, for example, by regulating the production of mRNA from DNA, such as chromosomal DNA or cDNA. In some embodiments, an actuator moiety recruits at least one transcription factor that binds to a specific DNA sequence, thereby controlling the rate of transcription of genetic information from DNA to mRNA. An actuator moiety can itself bind to DNA and regulate transcription by physical obstruction, for example preventing proteins such as RNA polymerase and other associated proteins from assembling on a DNA template. An actuator moiety can regulate expression of a gene at the translation level, for example, by regulating the production of protein from mRNA template. In some embodiments, an actuator moiety regulates gene expression at a post-transcriptional level by affecting the stability of an mRNA transcript. In some embodiments, an actuator moiety regulates gene expression at a post-translational level by altering the polypeptide modification, such as glycosylation of newly synthesized protein. In some embodiments, an actuator moiety regulates expression of a gene by editing a nucleic acid sequence (e.g., a region of a genome). In some embodiments, an actuator moiety regulates expression of a gene by editing an mRNA template. Editing a nucleic acid sequence can, in some cases, alter the underlying template for gene expression.

The actuator moiety may comprise a Cas protein or a modification thereof. A Cas protein referred to herein can be a type of protein or polypeptide. A Cas protein can refer to a nuclease. A Cas protein can refer to an endoribonuclease. A Cas protein can refer to any modified (e.g., shortened, mutated, lengthened) polypeptide sequence or homologue of the Cas protein. A Cas protein can be codon optimized. A Cas protein can be a codon-optimized homologue of a Cas protein. A Cas protein can be enzymatically inactive, partially active, constitutively active, fully active, inducible active and/or more active, (e.g. more than the wild type homologue of the protein or polypeptide.). A Cas protein can be Cas9. A Cas protein can be Cpf1. A Cas protein can be C2c2. A Cas protein can be Cas13a. A Cas protein can be Cas12, or a functional variant thereof. A Cas protein can be Cas12e. A Cas protein (e.g., variant, mutated, enzymatically inactive and/or conditionally enzymatically inactive site-directed polypeptide) can bind to a target nucleic acid. A Cas protein (e.g., variant, mutated, enzymatically inactive and/or conditionally enzymatically inactive endoribonuclease) can bind to a target RNA or DNA.

The terms “deactivated nuclease” and “dead nuclease,” as used interchangeably herein, generally refer to a nuclease, wherein the function of the nuclease is entirely or partially deactivated. In a case where the nuclease is a Cas protein, a deactivated/dead Cas nuclease may be referred to as “dCas” (e.g., dCas9).

The term “crRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can generally refer to a nucleic acid with at most about 5%, 10%, 20%, 30%, 40%, 50%, 60%, 70%, 80%, 90%, or 100% sequence identity and/or sequence similarity to a wild type exemplary crRNA (e.g., a crRNA from S. pyogenes, S. aureus, etc.). crRNA can refer to a modified form of a crRNA that can comprise a nucleotide change such as a deletion, insertion, or substitution, variant, mutation, or chimera. A crRNA can be a nucleic acid having at least about 60% sequence identity to a wild type exemplary crRNA (e.g., a crRNA from S. pyogenes, S. aureus, etc) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100 % identical to a wild type exemplary crRNA sequence (e.g., a crRNA from S. pyogenes S. aureus, etc) over a stretch of at least 6 contiguous nucleotides.

The term “tracrRNA,” as used herein, generally refers to a nucleic acid with at least about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA can refer to a nucleic acid with at most about 5%, 10%, 20%, 30%, 40%, 50%, 60%, 70%, 80%, 90%, or 100% sequence identity and/or sequence similarity to a wild type exemplary tracrRNA sequence (e.g., a tracrRNA from S. pyogenes S. aureus, etc). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes S. aureus, etc) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100 % identical to a wild type exemplary tracrRNA (e.g., a tracrRNA from S. pyogenes S. aureus, etc) sequence over a stretch of at least 6 contiguous nucleotides.

As used herein, a “guide nucleic acid” generally refers to a nucleic acid that can hybridize to another nucleic acid. A guide nucleic acid can be RNA. A guide nucleic acid can be DNA. The guide nucleic acid can be programmed to bind to a sequence of nucleic acid site-specifically. The nucleic acid to be targeted, or the target nucleic acid, can comprise nucleotides. The guide nucleic acid can comprise nucleotides. A portion of the target nucleic acid can be complementary to a portion of the guide nucleic acid. The strand of a double-stranded target polynucleotide that is complementary to and hybridizes with the guide nucleic acid can be called the complementary strand. The strand of the double-stranded target polynucleotide that is complementary to the complementary strand, and therefore may not be complementary to the guide nucleic acid can be called noncomplementary strand. A guide nucleic acid can comprise a polynucleotide chain and can be called a “single guide nucleic acid.” A guide nucleic acid can comprise two polynucleotide chains and can be called a “double guide nucleic acid.” If not otherwise specified, the term “guide nucleic acid” can be inclusive, referring to both single guide nucleic acids and double guide nucleic acids.

A guide nucleic acid can comprise a segment that can be referred to as a “nucleic acid-targeting segment” or a “nucleic acid-targeting sequence.” A nucleic acid-targeting segment can comprise a sub-segment that can be referred to as a “protein binding segment” or “protein binding sequence” or “Cas protein binding segment”.

The terms “cleavage recognition sequence” and “cleavage recognition site,” as used herein, with reference to peptides, refers to a site of a peptide at which a chemical bond, such as a peptide bond or disulfide bond, can be cleaved. Cleavage can be achieved by various methods. Cleavage of peptide bonds can be facilitated, for example, by an enzyme such as a protease

The term “targeting sequence,” as used herein, generally refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a GMP) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

The term “nuclear export signal (NES),” as used herein, generally refers to an amino acid sequence capable of direct a polypeptide containing it (such as a NES-containing chimeric polypeptide) to be exported from the nucleus of a cell. In some cases, such export may be mostly mediated by one or more proteins (e.g., one or more exportin proteins, such as chromosomal region maintenance 1 (Crm1)). In some cases, the NES may be rich in hydrophobic amino acid residues, such as leucine (Leu). Other examples of hydrophobic residues can include one or more of: glycine (Gly), alanine (Ala), valine (Val), isoleucine (Ile), proline (Pro), phenylalanine (Phe), methionine (Met), tryptophan (Trp), modifications thereof, and combinations thereof. In some examples, a leucine-rich NES may be a motif (e.g., a conservative or non-conservative motif) comprising 3 or 4 hydrophobic residues. In some examples, a NES motif may comprise a polynucleotide pattern LxxLxL, LxxxLxL, or LxxxLxxLxL, wherein each L is independently selected from the hydrophobic resides (e.g., leucine, isoleucine, valine, phenylalanine and methionine), and each x is independently selected from any amino acid. The NES may have a net positive charge. As an alternative, the NES may have a net negative charge. In a different alternative, the NES may have a net neutral charge.

As used herein, “fusion” generally refers to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

Thus, in some embodiments, an actuator moiety may comprise a fusion polypeptide. The fusion polypeptide may comprise two or more fragments that each confer at least one activity selected from the group consisting of: nuclease activity, methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity.

In some cases, the actuator moiety may comprise a fusion polypeptide, and the fusion polypeptide may comprise two fragments that each confer (i) a nuclease activity (or modifications thereof, e.g., Cas activity or reduced Cas activity) and (ii) a hydrolase activity (e.g., cytidine deaminase activity). In some examples, the actuator moiety comprising the fusion polypeptide may be a nucleobase editor. The term “nucleobase editor” or “base editor,” as used interchangeably herein, can refer to an agent comprising a polypeptide that is capable of making a modification to a nucleobase (e.g., A, T, C, G, or U) within a nucleic acid sequence (e.g., DNA or RNA). In some cases, the base editor (e.g., deaminase) may be capable of deaminating a base within a nucleic acid. In some cases, the base editor may be capable of deaminating a base within a DNA molecule. In some cases, the base editor may be capable of deaminating a cytosine (C) in DNA. In some cases, the base editor may be capable of excising a base within a DNA molecule. In some cases, the base editor may be capable of excising an adenine, guanine, cytosine, thymine or uracil within a nucleic acid (e.g., DNA or RNA) molecule. In some cases, the base editor may be a fusion protein comprising a programmable nucleic acid binding protein (e.g., a nuclease as provided in the present disclosure, such as Cas or dCas) fused to a cytidine deaminase. In some cases, the base editor may be fused to a uracil binding protein (UBP), such as a uracil DNA glycosylase (UDG). In some cases, the base editor may be fused to a nucleic acid polymerase (NAP) domain. In some cases, the NAP domain may be a translesion DNA polymerase. In some cases, the base editor may comprise a programmable nucleic acid binding protein, a cytidine deaminase, and a UBP (e.g., UDG). In some cases, the base editor may comprise a programmable nucleic acid binding protein, a cytidine deaminase, and a nucleic acid polymerase (e.g., a translesion DNA polymerase). In some cases, the base editor comprises a programmable nucleic acid binding protein, a cytidine deaminase, a UBP (e.g., UDG), and a nucleic acid polymerase (e.g., a translesion DNA polymerase).

In some examples, the base editor may introduce one or more transition mutations (e.g., C to T, G to A, A to G, or T to C) without requiring double stranded breaks in many cell types and organisms, including mammals.

In some cases, the actuator moiety may comprise a fusion polypeptide, and the fusion polypeptide may comprise two fragments that each confer (i) a nuclease activity (or modifications thereof, e.g., Cas activity or reduced Cas activity) and (ii) a polymerase activity (e.g., DNA or RNA polymerase activity). As used here, the term “polymerase” can refer to a polypeptide that is able to catalyze addition of one or more nucleotides or analogs thereof (e.g., natural or synthetic nucleotides) to a nucleic acid molecule in a template dependent manner. In an example, an DNA insertion sequence encoded by a template RNA molecule may be added to a 3′-end of a target DNA molecule by action of a polymerase (e.g., reverse transcriptase). Examples of a polymerase may include, but are not limited to, (i) polymerases isolated from Thermus aquaticus, Thermus thermophilus, Pyrococcus woesei, Pyrococcus furiosus, Thermococcus litoralis, and Thermotoga maritima, (ii) E. coli DNA polymerase I, the Klenow fragment of E. coli DNA polymerase I, T4 DNA polymerase, T5 DNA polymerase, T7 DNA polymerase, (iii) T7, T3, SP6 RNA polymerases, and (iv) AMV, M-MLV and HIV reverse transcriptase.

In some examples, the actuator moiety may comprise a fusion polypeptide, and the fusion polypeptide may comprise (i) a Cas protein or modifications thereof (e.g., deactivated Cas or Cas nickase) that is coupled (e.g., covalently coupled) to (ii) a reverse transcriptase. The Cas protein may be configured to only nick one strand of a target nucleic acid (e.g., one strand of a double stranded DNA molecule). The reverse transcriptase may be configured to generate a new nucleic acid sequence (e.g., a new DNA polynucleotide stand) by coping from a nucleic acid template (e.g., a RNA template). Such actuator moiety may function in conjunction with an engineered gRNA (i.e. prime editing gRNA, or pegRNA). The pegRNA may comprise a plurality of segments. The plurality of segments may comprise (i) a nucleic acid-targeting segment (e.g., spacer region of a gRNA), (ii) a Cas protein-binding segment (e.g., as two separate crRNA and tracrRNA molecules, or as a single scaffold molecule), (iii) a reverse transcriptase template segment encoding a desired nucleic acid edit, and (iv) a binding segment that binds to the nicked strand of the target nucleic acid. In an example, the reverse transcriptase template segment of the pegRNA may encode a desired DNA sequence. Alternatively, the reverse transcriptase template segment of the pegRNA may encode a complimentary DNA sequence having complementarity to a desired DNA sequence, such that when the complimentary DNA sequence is introduced to a first strand of the target gene, the desired DNA sequence may be subsequently added to a second and opposite strand of the target gene (e.g., via one or more DNA repair mechanisms).

In an example, a fusion complex of (i) an actuator moiety comprising the Cas protein and the reverse transcriptase and (ii) a pegRNA may introduce one or more transition mutations (e.g., C to T, G to A, A to G, or T to C) without requiring double stranded breaks in many cell types and organisms, including mammals. Alternatively or in addition to, such fusion complex may perform one or more transversion mutations (e.g., C to A, C to G, G to C, G to T, A to C, A to T, T to A, and T to G), e.g., for T-A to A-T mutation needed to correct sickle cell disease, without requiring double stranded breaks in many cell types and organisms, including mammals. Alternatively or in addition to, such fusion complex may introduce an indel (e.g., an insertion and/or deletion) to the target nucleic acid or target gene. The fusion complex may introduce an addition of at least 1, 2, 3, 4, 5, 6, 7, 8, 9, 10, 15, 20, 25, 30, 35, 40, 45, 50, 55, 60, 65, 70, 75, 80, 85, 90, or more nucleotides to the target gene. The fusion complex may introduce an addition of at most 90, 85, 80, 75, 70, 65, 60, 55, 50, 45, 40, 35, 30, 25, 20, 15, 10, 9, 8, 7, 6, 5, 4, 3, 2, or 1 nucleotide to the target gene. The fusion complex may introduce a deletion of at least 1, 2, 3, 4, 5, 6, 7, 8, 9, 10, 15, 20, 25, 30, 35, 40, 45, 50, 55, 60, 65, 70, 75, 80, 85, 90, or more nucleotides to the target gene. The fusion complex may introduce a deletion of at most 90, 85, 80, 75, 70, 65, 60, 55, 50, 45, 40, 35, 30, 25, 20, 15, 10, 9, 8, 7, 6, 5, 4, 3, 2, or 1 nucleotide to the target gene. The fusion complex may or may not introduce a frameshift in the gene.

In some cases, an engineered gRNA (e.g., a pegRNA) may be coupled (e.g., covalently or non-covalently coupled) to a moiety (e.g., a polypeptide molecule) that confers at least one activity selected from the group consisting of: nuclease activity, methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, and demyristoylation activity. In an example, a pegRNA may be operatively coupled to a nucleic acid polymerase (e.g., a reverse transcriptase) by action of the nucleic acid polymerase recognizing and non-covalently binding to a fragment (e.g., a loop structure) of the pegRNA. In such a case, the nucleic acid polymerase may or may not be covalently coupled to a nuclease (e.g., a Cas protein or a dCas protein).

As used herein, the term “non-native” generally refers to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The terms “subject,” “individual,” and “patient” are used interchangeably herein to generally refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, generally refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “chimeric antigen receptor” or alternatively a “CAR” may be used herein to generally refer to a recombinant polypeptide construct comprising at least an extracellular antigen binding domain, a transmembrane domain, and a cytoplasmic signaling domain (also referred to herein as “an intracellular or intrinsic signaling domain”) comprising a functional signaling domain derived from a stimulatory molecule. In some cases, the stimulatory molecule may be the zeta chain associated with the T cell receptor complex. In some cases, the intracellular signaling domain further comprises one or more functional signaling domains derived from at least one costimulatory molecule. In some cases, the costimulatory molecule may comprise 4-1BB (i.e., CD137), CD27, and/or CD28. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane. In some cases, the CAR may further comprise a GMP, as described in the present disclosure.

The CAR, as used herein, may be a first-, second-, third-, or fourth-generation CAR system, a functional variant thereof, or any combination thereof. First- generation CARs (e.g., CD19R or CD19CAR) include an antigen binding domain with specificity for a particular antigen (e.g., an antibody or antigen-binding fragment thereof such as an scFv, a Fab fragment, a VHH domain, or a VH domain of a heavy-chain only antibody), a transmembrane domain derived from an adaptive immune receptor (e.g., the transmembrane domain from the CD28 receptor), and a signaling domain derived from an adaptive immune receptor (e.g., one or more (e.g., three) ITAM domains derived from the intracellular region of the CD3 ζ receptor or FcεRIγ). Second-generation CARs modify the first-generation CAR by addition of a co-stimulatory domain to the intracellular signaling domain portion of the CAR (e.g., derived from co-stimulatory receptors that act alongside T-cell receptors such as CD28, CD137/4-1BB, and CD134/OX40), which abrogates the need for administration of a co-factor (e.g., IL-2) alongside a first-generation CAR. Third-generation CARs add multiple co-stimulatory domains to the intracellular signaling domain portion of the CAR (e.g., CD3ζ-CD28-OX40, or CD3ζ-CD28-41BB). Fourth-generation CARs modify second- or third-generation CARs by the addition of an activating cytokine (e.g., IL-12, IL-23, or IL-27) to the intracellular signaling portion of the CAR (e.g., between one or more of the costimulatory domains and the CD3ζ ITAM domain) or under the control of a CAR-induced promoter (e.g., the NFAT/IL-2 minimal promoter).

The term “conditionally enhancing expression” generally refers to expression of a polypeptide sequence (e.g., an endogenous polypeptide sequence, a chimeric polypeptide sequence, etc.) that occurs subject to one or more requirements rather than continually. Upon increasing, maintaining, and/or decreasing of the expression of the polypeptide sequence in a cell (e.g., an immune cell, a stem cell, etc.), the cell may be contacted with a stimulant (e.g., a ligand or an antigen) to initiate the conditional enhancement of expressing the polypeptide sequence in the cell. In some cases, the cell may not have begun expression the polypeptide sequence prior to at least a first contact with the stimulant. In some cases, the cell may have begun expression of the polypeptide sequence, and after the expression of the polypeptides sequence is plateaued out or decreased, the cell may be contacted with the stimulant to initiate the conditional enhancement of expressing the polypeptide sequence in the cell. The cell may be ex vivo (e.g., in vitro) or in vivo (e.g., administered to a subject). In some cases, the conditional enhancement of expressing the polypeptide sequence in the cell may be temporary or permanent. In some cases, the cell may be contacted with the stimulant at least about 1, 2, 3, 4, 5, 6, 7, 8, 9, 10 times, or more. In some cases, the cell may be contacted with the stimulant at most about 10, 9, 8, 7, 6, 5, 4, 3, 2, or 1 time.

In some cases, a continual expression of a polypeptide sequence (e.g., Cas, dCas, or a different protein that is endogenous or exogenous to the cell) may have an off-target effect on a host cell, e.g., cell cytotoxicity. In such a case, conditionally promoting and/or enhancing expression of the polypeptide sequence (e.g., via contacting the cell with a stimulant) may be beneficial, at least for a reason that cell cytotoxicity may be controlled (e.g., diminished or prevented). Alternatively or in addition to, conditionally promoting and/or enhancing expression of the polypeptide sequence may be beneficial in that a continual metabolic burden of the host cell to synthesize the polypeptide sequence can be controlled (e.g., diminished or prevented). Without wishing to be bound by theory, controlling the metabolic burden of the host cell can improve viability, proliferation, and/or function of the host cell.

The terms “operatively linked” and “under the operative control” may be used herein interchangeably to generally refer to two sequences (e.g., two nucleotide sequences, two polypeptide sequences, a nucleotide sequence and a polypeptide sequence) that are either physically linked or are functionally linked so that at least one of the sequences can act on the other sequence. In some cases, a gene regulatory sequence (e.g., a promoter) and an additional nucleotide sequence (e.g., a gene of interest, a transgene, etc.), are operatively linked if the expression (e.g., transcription and translation) of the additional nucleotide sequence can be governed by the gene regulatory sequence. Accordingly, the gene regulatory sequence and the additional nucleotide sequence to be expressed may be physically linked to each other, e.g., by inserting the gene regulatory sequence at or adjacent to a 5′ end of the additional nucleotide sequence to be expressed. Alternatively, the gene regulatory sequence and the additional nucleotide sequence to be expressed may be merely in physical proximity so that the gene regulatory sequence is functionally linked to the additional nucleotide sequence to be expressed. In some cases, the two sequences that are operatively linked may be separated by at least 5, 10, 20, 40, 60, 80, 100, 300, 500, 1500 bp, or more. In some cases, the two sequences that are operatively linked may be separated by at most 1500, 500, 300, 100, 80, 60, 40, 20, 10, 5 bp, or less.

The term “promoter” may be used herein to generally refer to the regulatory DNA region which controls transcription or expression of a gene and which can be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

The term “2A peptide” may generally refer to a class of viral oligopeptides (e.g., 18-22 amino-acid (aa)-long viral oligopeptides) that mediate “cleavage” of polypeptides during translation in cells (e.g., eukaryotic cells). The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” is believed to be ribosome skipping the formation of a glycyl-prolyl peptide bond at the C-terminus of the 2A sequence.

The term “toxin,” as used herein, generally refers to an anticellular agent (e.g., cytotoxins). Examples of the toxin may include, but is not limited to, a plant toxin, a fungal toxin, a bacterial toxin, a ribosome inactivating protein (RIP), a functional variant thereof, or a combination thereof. Additional examples of the toxin may include, but are not limited to, Abrin A chain, Diphtheria Toxin (DT) A-Chain, Pseudomonas exotoxin, RTA, Shiga Toxin A chain, Shiga-like toxin, Gelonin, Momordin, Pokeweed Antiviral Protein, Saporin, Trichosanthin, Barley toxin, functional variants thereof, and combinations thereof.

### Chimeric Polypeptides, Polynucleotides Thereof, and Compositions Thereof

In an aspect, the present disclosure provides a chimeric polypeptide comprising at least one nuclear export signal (NES) (e.g., at least one heterologous NES) linked to an adaptor protein of a receptor. The adaptor protein may be capable of directly binding to the receptor prior to, during, and/or subsequent to signaling of the receptor (e.g., in a cell). Alternatively or in addition to, the adaptor protein may not be capable of directly binding to the receptor prior to, during, and/or subsequent to signaling of the receptor. In some cases, the adaptor protein may indirectly bind the receptor via one or more binding moieties (e.g., a small molecule, a polypeptide, a polynucleotide, etc.) that are, individually or collectively, capable of binding both the adaptor and the receptor.

In some cases, the receptor may be introduced (e.g., expressed) in a cell. The receptor may be endogenous to the cell. Alternatively, the receptor may be heterologous to the cell. In some cases, the receptor may comprise a transmembrane receptor and/or nuclear membrane receptor. In some cases, the receptor may comprise a chimeric antigen receptor (CAR) and/or a T cell receptor (TCR).

In some cases, the chimeric polypeptide may comprise at least 1, 2, 3, 4, 5, 6, 7, 8, 9, 10, or more NES domains. In some cases, the chimeric polypeptide may comprise at most 10, 9, 8, 7, 6, 5, 4, 3, 2, or 1 NES domain. In some cases, the chimeric polypeptide may comprise a plurality of NES domains that are the same. In some cases, the chimeric polypeptide may comprise a plurality of NES domains that are different. In some cases, the chimeric polypeptide may comprise at least 1, 2, 3, 4, 5, 6, 7, 8, 9, 10, or more heterologous NES domains. In some cases, the chimeric polypeptide may comprise at most 10, 9, 8, 7, 6, 5, 4, 3, 2, or 1 heterologous NES domain. In some cases, the chimeric polypeptide may comprise a plurality of heterologous NES domains that are the same. In some cases, the chimeric polypeptide may comprise a plurality of heterologous NES domains that are different.

In some cases, the at least one heterologous NES may be linked to C-terminus and/or N-terminus of the adaptor protein. In some cases, a first heterologous NES may be linked to C-terminus of the adaptor protein, and a second heterologous NES may be linked to N-terminus of the adaptor protein. In such cases, the adaptor protein may be flanked by the first heterologous NES and the second heterologous NES.

The adaptor protein may be an entirely of the adaptor protein. In some cases, the adaptor protein may be a fragment thereof or a functional variant thereof. In some cases, the adaptor protein may be a fragment thereof and a functional variant thereof. In some cases, the adaptor protein may be selected from the group consisting of: 14-3-3 beta, 14-3-3 gamma, 14-3-3 epsilon, 14-3-3 eta, 14-3-3 sigma, 14-3-3 theta, 14-3-3 zeta, ALX, APBA2, APBA3, APPL, BAP31, BCAP, BLNK, BRDG1, CARD9, CBL, CD2AP, CHIP/STUB1, CIDEA, CIDEC, CIN85/SH3KBP1, CRADD, Crk, CrkL, DAB2, DAP10/HCST, DAPP1, Daxx, DFF40/CAD, DFF45/ICAD, DISC1, DOCK1, DOCK2, DOCK3, DOK3, DOK7, Erbin, FADD, FLIP, FRS2, GAB2, GAB3, GAPDH, GAPDH-2, GBL, GRAP2, GRB2, GRB7, GULP1/CED-6, Importin alpha 2/KPNA2, Importin alpha 3/KPNA4, Importin alpha 5/KPNA1,Importin beta/KPNB1, IRS1, IRS2, a Linker for Activation of T cells (LAT), LNK/SH2B3, Mena, MIG2, MyD88, NCK1, NOD1, NTAL, Numb, p130Cas, p62/SQSTM1, PAG1, PARD3/Par3, Paxillin, PDHX, PINCH1, Ras-GAP, RIAM/APBB1IP, SAM68, SH2B1, SH2D1A, SH2D2A, SHANK2, SHB, SHC1, SIT1, SOCS-5, SOCS-6, SOCS-7/Nck/NAP4, STAM-1, STI1, SWAP70, TANK, TAPP1, Tollip, TRADD, TRAF-1, TRAF-2, TRAF-3, TRAF-4, TRAF-5, TRAF-6, TRAM/TICAM2, TRIF/TICAM1, TRRAP, UBASH3B/STS1, Vav-1, ARRB2, c-Cbl, GADS, Lck, SLP-76, ZAP-70, Sos1, plc-γ1, PI3K, DAG, a fragment thereof, a functional variant thereof, and a combination thereof.

In some cases, the adaptor protein may be a fusion or a collection (e.g., non-fused collection) of a plurality of adaptor proteins. The plurality of adaptor proteins may comprise the same adaptor proteins or different adaptor proteins. In some cases, the adaptor protein may comprise at least the LAT. In some cases, the LAT may comprise at least one isoform of the LAT. Alternatively or in addition to, the LAT may comprise a functional variant of the LAT.

In some cases, upon introduction of the chimeric polypeptide into a cell comprising the receptor, the chimeric polypeptide may prolong or enhance signaling (e.g., intracellular signaling) of the receptor in the cell, as compared to the adaptor protein without the at least one heterologous NES. In some examples, the prolonged signaling may be characterized by chemical modification (e.g., phosphorylation) of one or more signaling cascade proteins of the receptor for a longer period of time as compared to the adaptor protein without the at least one heterologous NES. In some examples, the enhanced signaling may be characterized by a higher expression one or more signaling cascade proteins of the receptor or target genes of the receptor as compared to the adaptor protein without the at least one heterologous NES. In some examples, the prolonged or enhanced signaling may be characterized by delayed degradation of the receptor. In some examples, the prolonged or enhanced signaling may be characterized by prolonged or enhanced activity of the cell (e.g., survival, proliferation, differentiation, migration, endosomal activity, metabolic activity, cytotoxicity against a target cell, etc.).

In some cases, the at least one heterologous NES may (i) enhance translocation of the adaptor protein into a membrane of the cell, (ii) reduce displacement of the adaptor protein from a membrane of the cell during the signaling of the receptor, (iii) reduce degradation of the adaptor protein during the signaling of the receptor, or (iv) stabilize the adaptor protein in a membrane of the cell, as compared to the adaptor protein without the at least one heterologous NES. In some cases, the at least one heterologous NES may (i) enhance translocation of the adaptor protein into a membrane of the cell, (ii) reduce displacement of the adaptor protein from a membrane of the cell during the signaling of the receptor, (iii) reduce degradation of the adaptor protein during the signaling of the receptor, and (iv) stabilize the adaptor protein in a membrane of the cell, as compared to the adaptor protein without the at least one heterologous NES. In some examples, the at least one heterologous NES may at least enhance translocation of the adaptor protein into a membrane of the cell. In some examples, the at least one heterologous NES may at least reduce displacement of the adaptor protein from a membrane of the cell during the signaling of the receptor.

The at least one heterologous NES may comprise at least two amino acid residues that include at least one hydrophobic amino acid residue. In some cases, the at least one heterologous NES may comprise at least two hydrophobic amino acid residues and at least one additional amino acid residue that is flanked by the at least two hydrophobic amino acid residues. In some cases, the at least one heterologous NES may comprise a polynucleotide pattern comprising LxxLxL, LxxxLxL, and/or LxxxLxxLxL, wherein each L is a hydrophobic amino acid residue. In some cases, the hydrophobic amino acid residue may be selected from the group consisting of leucine, isoleucine, valine, phenylalanine, and methionine, a functional variant thereof, and a combination thereof. In some cases, the hydrophobic amino acid residue may be natural or synthetic. In some examples, a sequence of the at least one NES may be LALKLAGLDI (SEQ ID NO: 2), LQLPPLERLTL (SEQ ID NO:3), a functional variant thereof, or a combination thereof. In an example, the sequence of the at least one NES may be LALKLAGLDI (SEQ ID NO: 2). In another example, the sequence of the at least one NES may be LQLPPLERLTL (SEQ ID NO:3).

In some cases, a portion of the chimeric polypeptide encoding the adaptor protein may comprise at least one mutation, as compared to a wild-type adaptor protein of the cell or a different type of cell. The at least one mutation may be within the at least one heterologous NES. Alternatively, the at least one mutation may be outside of the at least one heterologous NES. In some cases, the at least one mutation may be an addition of at least one amino acid. The at least one mutation may be an addition of at least 1, 2, 3, 4, 5, 6, 7, 8, 9, 10, 15, 20, 30, 40, 50, or more amino acid residues. The at least one mutation may be an addition of at most 50, 40, 30, 20, 15, 10, 9, 8, 7, 6, 5, 4, 3, 2, or 1 amino acid residue. The at least one mutation may be a plurality of amino acid residues that are sequential or not. In some cases, the at least one mutation may be a deletion of at least one amino acid. The at least one mutation may be a deletion of at least 1, 2, 3, 4, 5, 6, 7, 8, 9, 10, 15, 20, 30, 40, 50, or more amino acid residues. The at least one mutation may be a deletion of at most 50, 40, 30, 20, 15, 10, 9, 8, 7, 6, 5, 4, 3, 2, or 1 amino acid residue. In some cases, the at least one mutation may be introduced to at least the adaptor protein of the chimeric polypeptide prior to or subsequent to the introduction of the at least one heterologous NES to the adaptor protein to provide the chimeric polypeptide. In some cases, the at least one mutation (e.g., a plurality of mutations) may be introduced to the adaptor protein prior to and subsequent to the introduction of the at least one heterologous NES to the adaptor protein to provide the chimeric polypeptide.

In some cases, the at least one mutation may be introduced at one or more residues of the adaptor protein that serves as at least a portion of a substrate for post-translational modification. As such, the at least one mutation may hinder or prevent one or more post-translational modifications of the adaptor protein. Examples of sites that may undergo post-translational modification may comprise one or more residues having a functional group that may serve as a nucleophile in the reaction: the hydroxyl groups of serine, threonine, and tyrosine; the amine forms of lysine, arginine, and histidine; the thiolate anion of cysteine; the carboxylates of aspartate and glutamate; and the N-and C-termini. Alternatively or in addition to, the amide of asparagine may serve as an attachment point for one or more polysaccharides (i.e., glycans). Additionally, or in addition to, one or more oxidized methionines and/or methylenes in side chains of amino acid residues may serve as post-translational modification substrate.

In some cases, the post-translational modification may be ubiquitination (e.g., attachment of one or more ubiquitin proteins to the adaptor protein at a ubiquitination substrate of the adaptor protein). In some examples, a bond (e.g., a covalent bond, such as an isopeptide bond) may be formed between a carboxyl group (COO⁻) of the ubiquitin’s glycine and the epsilon-amino group (ε-NH⁺₃) of the substrate’s residue, such as a lysine residue. In some cases, ubiquitination of the adaptor protein during the receptor signaling may serve as (i) a sorting signal that targets activated molecules (e.g., a ubiquitinated adaptor protein) at the cell surface for endocytosis and/or (ii) an intracellular sorting signal for molecules to be targeted for degradation instead of being recycled back to the cell membrane. Thus, the at least one mutation of the adaptor protein (e.g., at one or more post-translational modification sites, such as one or more ubiquitination sites) may reduce or prevent ubiquitination of the adaptor protein, reduce or prevent degradation of the adaptor protein, and prolong or enhance signaling (e.g., intracellular signaling) of the receptor.

In some cases, at least 1, 2, 3, 4, 5, 6, 7, 8, 9, 10, or more mutations may be introduced at or adjacent to a post-translational modification substrate (e.g., a lysine residue) of the adaptor protein. In some cases, at most 10, 9, 8, 7, 6, 5, 4, 3, 2, or 1 mutation may be introduced at or adjacent to a post-translational modification substrate of the adaptor protein. In some cases, at least 1, 2, 3, 4, 5, 6, 7, 8, 9, 10, or more post-translational modification substrates of the adaptor protein may be mutated. In some cases, at most 10, 9, 8, 7, 6, 5, 4, 3, 2, or 1 post-translational modification substrate of the adaptor protein may be mutated. In some cases, the at least one mutation may be at one or more lysine residues of the wild-type adaptor protein. In some cases, the wild-type adaptor protein may be a human (e.g., Homo sapiens) wild-type adaptor protein. Alternatively, the wild-type adaptor protein may not be a human wild-type adaptor protein. In some alternative examples, the wild-type adaptor protein may be from Mus musculus, Cricetulus griseus, Rattus Norvegicus, Danio rerio, or C elegans. In some cases, at least 1, 2, 3, 4, 5, 6, 7, 8, 9, 10, or more lysine residues of the adaptor protein may be mutated. In some cases, at most 10, 9, 8, 7, 6, 5, 4, 3, 2, or 1 lysine residue of the adaptor protein may be mutated. In some cases, the at least mutation may comprise (i) deletion of the one or more lysine residues, or (ii) substitution of the one or more lysine residues with one or more different amino acid residues that are natural or synthetic. In some cases, the at least mutation may comprise (i) deletion of the one or more lysine residues, and (ii) substitution of the one or more lysine residues with one or more different amino acid residues that are natural or synthetic. In some examples, the adaptor protein may be the LAT (e.g., human LAT), and the at least one mutation may comprise substitution of K52 and/or K204 lysine residues. In some examples, the lysine residue(s) may be substituted with a non-lysine residue, such as arginine or a variant thereof.

In some cases, upon introduction of the chimeric polypeptide into a cell comprising the receptor, the at least one mutation of the adaptor protein may (i) reduce displacement of the adaptor protein from a membrane of the cell during signaling of the receptor, or (ii) reduce ubiquitination of the adaptor protein during signaling of the receptor, as compared the wild-type adaptor protein without the at least one mutation. In some cases, upon introduction of the chimeric polypeptide into a cell comprising the receptor, the at least one mutation of the adaptor protein may (i) reduce displacement of the adaptor protein from a membrane of the cell during signaling of the receptor, and (ii) reduce ubiquitination of the adaptor protein during signaling of the receptor, as compared the wild-type adaptor protein without the at least one mutation.

In some cases, signaling of the receptor may induce oxidative stress (e.g., acute or chronic oxidative stress) in the cell and/or reduced intracellular levels of one or more antioxidants (e.g., glutathione (GSH), which may result in displacement of the adaptor protein (e.g., the LAT). Thus, a targeted mutation of one or more redox-sensitive amino acid residues (e.g., one or more cysteine-to-serine mutations) within the adaptor protein may help the adaptor protein to remain anchored in the membrane during the receptor signaling, as compared to the adaptor protein without the at least one mutation. Alternatively or in addition to, the at least one mutation may reduce of prevent a conformational change of the adaptor protein during the receptor signaling (e.g., during the oxidative stress from the receptor signaling) in comparison to the adaptor protein without the at least one mutation, thereby rending the adaptor protein insensitive to redox balance alternations in the cell.

In some cases, the at least one mutation may be at one or more thiol-containing residues (e.g., cysteine residues) of the wild-type adaptor protein. In some cases, the at least one mutation may comprise targeted mutation of at least 1, 2, 3, 4, 5, 6, 7, 8, 9, 10, or more cysteine-to-non-cysteine mutations. In some cases, the at least one mutation may comprise (i) deletion of the one or more cysteine residues, or (ii) substitution of the one or more cysteine residues with one or more different amino acid residues that are natural or synthetic. In some cases, the at least one mutation may comprise (i) deletion of the one or more cysteine residues, and (ii) substitution of the one or more cysteine residues with one or more different amino acid residues that are natural or synthetic. In some cases, the at least one mutation may comprise targeted mutation of at most 10, 9, 8, 7, 6, 5, 4, 3, 2, or 1 cysteine-to-non-cysteine mutation. In some examples, the adaptor protein may be the LAT, and the at least one targeted mutation may comprise substitution of one or more cysteines selected from the group consisting of C9, C26, C29, and C117.

In some cases, the at least one mutation may prevent or reduce the probability of a chemical modification of at least a portion of the adaptor protein (e.g., at or adjacent to a post-translational modification site), thereby preventing or reducing the probability of the post-translational modification. In some cases, the at least one mutation may alter structural conformation (e.g., folding) of the post-translational modification site, thereby preventing or reducing the probability of the post-translational modification. In some cases, the at least one mutation may prevent or reduce the probability of a structural modification of at least a portion of the adaptor protein, thereby preventing or reducing the probability of the post-translational modification.

In some cases, the chimeric polypeptide may comprise at least one additional residue (e.g., a single amino acid residue or a polypeptide). In some cases, upon introduction of the chimeric polypeptide into a cell, a charge, size, or position of the at least one additional polypeptide relative to the chimeric polypeptide may be sufficient to inhibit or reduce interaction between the adaptor protein and a cellular component of the cell. The cellular component may comprise a nucleic acid, a polynucleotide, an amino acid, a polypeptide, lipid, a carbohydrate, a small molecule, an enzyme, a ribosome, a proteasome, a variant thereof, or any combination thereof. In some cases, upon introduction of the chimeric polypeptide into the cell, at least two (e.g., 2 or 3) properties selected from the group consisting of: a charge, size, and position of the at least one additional polypeptide relative to the chimeric polypeptide may be sufficient to inhibit or reduce interaction between the adaptor protein and the cellular component of the cell. The interaction may be direct or indirect binding (or complexing). In some cases, the binding can be covalent (e.g., disulfide bond) or noncovalent (e.g., hydrogen bond). In some cases, the adaptor protein and the additional polypeptide may require a coupling moiety. The coupling moiety may bind to (i) at least a portion of the chimeric polypeptide (that is not the at least one additional polypeptide) and (ii) at least a portion of the at least one additional polypeptide, such that the at least one additional polypeptide is indirectly complexed to the chimeric polypeptide. The coupling moiety may be a small molecule, polynucleotide, polypeptide, a particle (e.g., nanoparticles), a functional variant thereof, or a combination thereof.

In some cases, the at least one additional polypeptide may be a single polypeptide. Alternatively or in addition to, the at least one additional polypeptide may comprise a plurality of polypeptides that are not connected. In some examples, a first additional polypeptide may be introduced (e.g., inserted) into a first site of the chimeric polypeptide, and a second additional polypeptide may be introduced (e.g., inserted) to a second site of the chimeric polypeptide, wherein the first and second sites are different and are not adjacent to one another. In some cases, the at least one additional polypeptide may comprise a plurality of amino acid residues. The plurality of amino acid residues may comprise at least 2, 3, 4, 5, 6, 7, 8, 9, 10, 15, 20, 30, 40, 50, or more amino acid residues. The plurality of amino acid residues may comprise at most 50, 40, 30, 20, 15, 10, 9, 8, 7, 6, 5, 4, 3, or 2 amino acid residues.

In some cases, the at least one additional polypeptide may prevent or reduce degradation of at least a portion of the adaptor protein in the cell, as compared to the adaptor protein without the at least one additional polypeptide. In some cases, the at least one additional polypeptide may prolong or improve half-life of the adaptor protein in the cell, as compared to the adaptor protein without the at least one additional polypeptide. In some cases, a detected amount of the adaptor protein in the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to the adaptor protein without the at least one additional polypeptide. In some cases, a detected amount of the adaptor protein in the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to the adaptor protein without the at least one additional polypeptide.

In some cases, the at least one additional polypeptide may be disposed at an extracellular portion, a membrane portion, or an intracellular portion of the chimeric polypeptide. In some cases, the at least one additional polypeptide may be disposed at an extracellular portion, a membrane portion, and an intracellular portion of the chimeric polypeptide. In some cases, the at least one additional polypeptide may be disposed at an extracellular portion of the chimeric polypeptide. In some cases, the at least one additional polypeptide may be disposed at a membrane portion (e.g., transmembrane or nuclear membrane) of the chimeric polypeptide. In some cases, the at least one additional polypeptide may be disposed at an intracellular portion of the chimeric polypeptide. In some cases, the at least one additional polypeptide may be disposed at an intracellular portion of the chimeric polypeptide. In some cases, the at least one additional polypeptide may be disposed at or adjacent to (i) C-terminus or (ii) N-terminus of the chimeric polypeptide. In some cases, the at least one additional polypeptide may be disposed at or adjacent to (i) C-terminus and (ii) N-terminus of the chimeric polypeptide. In some cases, (i) the at least one additional polypeptide may be flanked by the adaptor protein and the at least one heterologous NES, or (ii) the at least one heterologous NES may be flanked by the adaptor protein and the at least one additional polypeptide.

In some cases, the chimeric polypeptide may further comprise a recognition moiety that is specifically recognized by a capture moiety. The capture moiety may be a small molecule, lipid, polynucleotide, polypeptide, a variation thereof, or a combination thereof. The capture moiety may be naturally derived, synthetic, or a combination thereof. In some examples, the capture moiety may comprise a protein, e.g., an antibody. In an example, the chimeric polypeptide may further comprise the recognition moiety that may be specifically recognized by an antibody. The antibody may be an antibody or antigen-binding fragment thereof such as an scFv, a Fab fragment, a VHH domain, or a VH domain of a heavy-chain only antibody. The capture moiety may covalently and/or non-covalently bind to at least a portion of the recognition moiety. In some cases, the capture moiety may be part of an additional cell (e.g., exposed on a surface of the additional cell) that is different than the cell comprising the chimeric polypeptide provided herein. Alternatively or in addition to, the capture moiety may not be part of such additional cell. In some cases, the capture moiety may bind both the cell comprising the chimeric polypeptide and the additional cell.

In some cases, upon introduction of the chimeric polypeptide into the cell, contacting of the recognition moiety by the capturing moiety (e.g., an antibody or a functional variant thereof) may promote or enhance (i) antibody-dependent cellular cytotoxicity (ADCC), or (ii) complement-dependent cytotoxicity (CDC) of the cell. In some cases, upon introduction of the chimeric polypeptide into the cell, contacting of the recognition moiety by the capturing moiety (e.g., an antibody or a functional variant thereof) may promote or enhance (i) ADCC, and (ii) CDC of the cell. In the ADCC, one or more antibodies (e.g., of the same type or of different types) may bind one or more recognition moieties (e.g., one or more antigens) of the chimeric polypeptide of a cell. The one or more recognition moieties may be part of an extracellular portion of the chimeric polypeptide. Subsequently, an effector cell (e.g., a cell configured to lyse its target cell) may recognize and bind the one or more antibodies that are bound to the one or more recognition moieties. The complexing of the effector cell and the cell comprising the chimeric polypeptide may trigger the effector cell to lyse the cell. In some cases, the effector cell may induce death (e.g., via apoptosis) of the cell comprising the chimeric polypeptide. In the CDC, the cell comprising the chimeric polypeptide may be bound by one or more recognition moieties (e.g., one or more antibodies). The antibody-coated cell may trigger recruitment and activation of one or more components of the complement cascade, leading to a formation of a membrane attack complex (MAC) on the cell surface and subsequent cell lysis. In some cases, the recognition moiety may comprise epidermal growth factor receptor (EGFR), a fragment thereof, or a functional variant thereof. In some examples, the recognition moiety may be a truncated EGFR (tEGFR), truncated nerve growth factor receptor (tNGFR), low affinity NGFR (LNGFR), CD4, CD19, CD20, CD34, CD52, a fragment thereof, a functional variant thereof, or a combination thereof.

In some cases, the recognition moiety may not comprise any toxin capable of inducing death of the cell. Alternatively, the recognition moiety may comprise at least one toxin capable of inducing death of the cell. The recognition moiety may comprise at least 1, 2, 3, 4, 5, 6, 7, 8, 9, 10, or more toxins. The recognition moiety may comprise at most 10, 9, 8, 7, 6, 5, 4, 3, 2, or 1 toxin. In some cases, the antibody may comprise at least one toxin capable of inducing death of the cell. The antibody moiety may comprise at least 1, 2, 3, 4, 5, 6, 7, 8, 9, 10, or more toxins. The antibody may comprise at most 10, 9, 8, 7, 6, 5, 4, 3, 2, or 1 toxin. Examples of such toxin may include, but are not limited to, PK inhibitors (e.g., imatinib mesylate, gefitinib, dasatinib, erlotinib, lapatinib, sunitinib, nilotinib, and sorafenib; antibodies, including, e.g., trastuzumab, rituximab, cetuximab, and bevacizumab; mitoxantrone; dexamethasone; prednisone; and temozolomide), alkylating agents (e.g., melphalan, chlorambucil, busulfan, thiotepa, ifosfamide, carmustine, lomustine, semustine, streptozocin, decarbazine, and cyclophosphamide), mitotic inhibitors, antimetabolites (e.g., capecitibine, gemcitabine, 5-fiuorouracil or 5-fluorouracil/ leucovorin, fiudarabine, cytarabine, mercaptopurine, thioguanine, pentostatin, and methotrexate), cell cycle inhibitors, enzymes, hormones, anti-hormones, growth-factor inhibitors, plant alkaloids and terpenoids, topoisomerase inhibitors (e.g., etoposide, teniposide, camptothecin, topotecan, irinotecan, doxorubicin, and daunorubicin), antitumor antibiotics (e.g., actinomycin D, bleomycin, mitomycin C, adriamycin, daunorubicin, idarubicin, doxorubicin and pegylated liposomal doxorubicin), vinca alkaloids (e.g., vincristine and vinblastin), taxanes (e.g., paclitaxel and docetaxel), platinum agents (e.g., cisplatin, carboplatin, and oxaliplatin), thalidomide and related analogs (e.g., CC-5013 and CC-4047), antiangiogenic agents, functional variants thereof, and combinations thereof.

The recognition moiety capable of promoting or enhancing (i) ADCC or (ii) CDC of the cell upon contacting by the capturing moiety may be referred to as a “safety switch” to regulate survival or duration of the cell in a subject’s body. Introduction of the safety switch to the cell may increase safety profile and limit on-target or off-tumor toxicities of the cell (e.g., a chimeric antigen receptor T cell) in the subject’s body. In some cases, the safety switch may be part of the chimeric polypeptide of the present disclosure. Alternatively or in addition to, the safety switch may not be part of the chimeric polypeptide. The cell comprising the chimeric polypeptide may further comprise an inducible suicide gene encoding for one or more safety switches. The safety switch(es) may be assembled on a vector, such as, without limiting, a retroviral vector, lentiviral vector, adenoviral vector, or plasmid (e.g., a non-viral polynucleotide). The safety switch may be an inducible suicide gene, such as, without limiting, caspase 9 gene, thymidine kinase, cytosine deaminase (CD), cytochrome P450, a functional variant thereof, or a combination thereof. In an example, the suicide gene may be herpes simplex virus thymidine kinase (HSV-tk), which may convert a prodrug ganciclovir (GCV) into GCV-triphosphate, resulting in cell death by incorporation into replicating DNA. In another example, the suicide gene may be inducible caspase 9 (iCasp9), which may be a chimeric protein that binds a small molecule (e.g., AP1903), leading to caspase 9 dimerization and apoptosis of the cell. Additional examples of the safety switch for elimination of the cell (e.g., any unwanted modified T cells) may include the recognition moiety recognizable by the capturing moiety, as provided in the present disclosure (e.g., tEGFR that is recognizable by an anti-EGFR antibody, such as, for example, cetuximab). In some cases, any of the subject suicide genes may be integrated into the genome of the cell.

In another aspect, the present disclosure provides a polynucleotide encoding at least the chimeric polypeptide of any of the subject chimeric polypeptides provided herein. The polynucleotide may be introduced (e.g., inserted) as part of a genome of the cell. Alternatively or in addition to, the polynucleotide may be introduced to the cell, but not as part of the genome of the cell. In a different aspect, the present disclosure provides an expression cassette comprising at least the subject polynucleotide. The subject polynucleotide may be operatively linked to a regulatory sequence. The regulatory sequence may be endogenous to the cell. Alternatively or in addition to, the regulatory sequence may be heterologous to the cell.

In a different aspect, the present disclosure provides a composition comprising at least the subject expression cassette. The at least the subject expression cassette may be in a pharmaceutically acceptable carrier. The pharmaceutically acceptable carrier may be generally safe, non-toxic, and neither biologically nor otherwise undesirable, and may include a carrier acceptable for veterinary use as well as human pharmaceutical use. In some cases, the pharmaceutically acceptable carrier may comprise a pharmaceutically acceptable salt. The pharmaceutically acceptable salt may be derived from a variety of organic and inorganic counter ions, e.g., sodium, potassium, calcium, magnesium, ammonium, tetraalkylammonium, and combinations thereof; and salts of organic or inorganic acids, such as hydrochloride, hydrobromide, tartrate, mesylate, acetate, maleate, oxalate and combinations thereof.

In a different aspect, the present disclosure provides a kit comprising any one of the subject compositions. In a different aspect, the present disclosure provides a cell comprising at least any of the subject chimeric polypeptides provided herein. In some cases, the cell may further comprise the receptor. Examples of the cell are provided herein.

### Systems and Methods for Regulating Expression of a Target Polynucleotide

In a different aspect, the present disclosure provides a system for regulating signaling of a receptor in a cell. The system may comprise a chimeric polypeptide comprising an adaptor protein of a receptor linked to at least one NES (e.g., at least one heterologous NES). Upon introducing the system into the cell, the chimeric polypeptide may prolong or enhance the signaling of the receptor, as compared to the adaptor protein without the at least one heterologous NES. The chimeric polypeptide may comprise any one of the subject chimeric polypeptides of the present disclosure. The receptor may comprise any one of the subject receptors of the present disclosure. The at least one heterologous NES may comprise any one of the subject heterologous NES of the present disclosure.

In some cases, the chimeric polypeptide may comprise at least a portion of a transmembrane domain of the adaptor protein. In some cases, the chimeric polypeptide may comprise the transmembrane domain of the adaptor protein. In some cases, the chimeric polypeptide may comprise a transmembrane domain of another molecule (e.g., a different adaptor protein, a different receptor protein, etc.).

In some cases, the at least one heterologous NES may (i) enhance translocation of the adaptor protein into a membrane of the cell, (ii) reduce displacement of the adaptor protein from a membrane of the cell during the signaling of the receptor, (iii) reduce degradation of the adaptor protein during the signaling of the receptor, and/or (iv) stabilize the adaptor protein in a membrane of the cell, as compared to the adaptor protein without the at least one heterologous NES.

In some cases, the receptor may comprise a ligand binding domain specific for a ligand. The receptor may be activated upon binding of the ligand to the ligand binding domain. In some cases, the ligand may be an extracellular ligand. In some cases, the extracellular ligand may be an antigen presented on a target cell of the cell or released by the target cell of the cell. The antigen may be membrane bound. Alternatively or in addition to, the antigen may not be membrane bound, e.g., released from a target cell of the cell. Examples of antigens which can be bound by a ligand binding domain of the receptor are provided herein. In some cases, the chimeric polypeptide may prolong or enhance cytotoxicity of the cell against the target cell, as compared to the adaptor moiety without the at least one heterologous NES. In some cases, the chimeric polypeptide may prolong or enhance signaling of the receptor of the cell, thereby prolonging or enhancing cytotoxicity of the cell against the target cell. In some cases, the target cell may comprise a diseased cell, a tumor cell, and/or a cancer cell. In some cases, the chimeric polypeptide may reduce a size of or obliterates a tumor, as compared to the adaptor moiety without the at least one heterologous NES.

In some cases, the receptor may be exogenous or heterologous to the cell. In some cases, the heterologous receptor may be a chimeric receptor, such as, for example, a chimeric antigen receptor (CAR), as provided herein in the present disclosure. Examples of target antigens which can be bound by a ligand interacting domain of the CAR are provided herein. In some examples, the CAR may comprise at least a portion of a Notch receptor, a G-protein coupled receptor (GPCR), an integrin receptor, a cadherin receptor, a receptor tyrosine kinase, a death receptor, and/or an immune receptor. The immune receptor may comprise a T cell receptor (TCR). In some cases, the receptor may be endogenous to the cell. In some cases, the endogenous receptor may comprise a Notch receptor, a G-protein coupled receptor (GPCR), an integrin receptor, a cadherin receptor, a receptor tyrosine kinase, a death receptor, and/or an immune receptor. The immune receptor may comprise a T cell receptor (TCR).

In some cases, the system further comprises (i) a gene modulating polypeptide (GMP) comprising an actuator moiety linked to a cleavage recognition site, wherein the actuator moiety regulates the expression of the target polynucleotide in the cell, and (ii) a cleavage moiety capable of cleaving the cleavage recognition site of the GMP. In some cases, activation of the receptor may induce the cleavage moiety to cleave the cleavage recognition site, to effect regulating expression of the target polynucleotide in the cell.

In some cases, the receptor may comprise the GMP and the chimeric polypeptide may comprise the cleavage moiety. Alternatively, the chimeric polypeptide may comprise the GMP and the receptor may comprise the cleavage moiety. In some cases, the system may further comprise an additional polypeptide that comprises (i) the GMP and (ii) a coupling polypeptide configured to bind the receptor or a downstream signaling moiety of the receptor in response to the activation of the receptor. In such cases, the receptor may comprise the cleavage moiety. Alternatively, the chimeric polypeptide may comprise the cleavage moiety. In some cases, the system further comprises an additional polypeptide that comprises (i) the cleavage moiety and (ii) a coupling polypeptide configured to bind the receptor or a downstream signaling moiety of the receptor in response to the activation of the receptor. In such cases, the receptor may comprise the GMP. Alternatively, the chimeric polypeptide may comprise the GMP.

In a different aspect, the present disclosure provides a polynucleotide encoding at least a portion of any one of the subject systems provided herein. In a different aspect, the present disclosure provides an expression cassette comprising the subject polynucleotide. In the expression cassette, the at least the polynucleotide may be operatively linked to a regulatory sequence. The regulatory sequence may be endogenous to the cell. Alternatively or in addition to, the regulatory sequence may be heterologous to the cell.

In a different aspect, the present disclosure provides a composition comprising one or more polynucleotides that encode at least a portion of any one of the subject systems provided herein. The one or more polynucleotides may be in a pharmaceutically acceptable carrier. The pharmaceutically acceptable carrier may be generally safe, non-toxic, and neither biologically nor otherwise undesirable, and may include a carrier acceptable for veterinary use as well as human pharmaceutical use. In some cases, the pharmaceutically acceptable carrier may comprise a pharmaceutically acceptable salt. The pharmaceutically acceptable salt may be derived from a variety of organic and inorganic counter ions, e.g., sodium, potassium, calcium, magnesium, ammonium, tetraalkylammonium, and combinations thereof; and salts of organic or inorganic acids, such as hydrochloride, hydrobromide, tartrate, mesylate, acetate, maleate, oxalate and combinations thereof.

In a different aspect, the present disclosure provides a kit comprising any one of the subject compositions. In a different aspect, the present disclosure provides a cell comprising at least a portion of any one of the subject systems provided herein. The cell may be an isolated host cell expressing the at least the portion of any one of the subject systems provided herein. Examples of the cell (e.g., the isolated host cell) are provided herein. In some cases, the host cell may be an immune cell. The immune cell may be a lymphocyte. The lymphocyte may be a T cell. The T cell may be selected from the group consisting of: Cytotoxic T cell, Natural Killer T cell, Regulatory T cell, and T helper cell. Alternatively or in addition to, in some examples, the host cell may be a hematopoietic stem cell or an Induced pluripotent stem cell (iPSC).

In a different aspect, the present disclosure provides a method of enhancing signaling of a receptor in a cell. In some cases, the method may comprise expressing a system in the cell. The system may be any one of the subject systems provided herein, such as, for example, any one of the subject receptors (e.g., endogenous or CAR) provided herein, any one of the subject chimeric polypeptides provided herein, and/or any one of the subject GMP provided herein. In some cases, the system may comprise a chimeric polypeptide comprising an adaptor protein of a receptor linked to at least one nuclear export signal (NES) (e.g., at least one heterologous NES). In some cases, the chimeric polypeptide may enhance the signaling of the receptor, as compared to the adaptor protein without the at least one heterologous NES. In some cases, the enhanced signaling of the receptor may be evidenced by (i) enhanced viability of the cell, (ii) enhanced proliferation of the cell, (iii) enhanced intracellular signaling of the cell, (iv) enhanced cytotoxicity against a target cell, or (v) enhanced ability to reduce a size of or obliterate a tumor. In some cases, the enhanced signaling of the receptor may be evidenced by at least two or more of: (i) enhanced viability of the cell, (ii) enhanced proliferation of the cell, (iii) enhanced intracellular signaling of the cell, (iv) enhanced cytotoxicity against a target cell, and (v) enhanced ability to reduce a size of or obliterate a tumor.

In some cases, the enhanced signaling may be evidenced by enhanced viability of the cell. In some cases, the viability of the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the viability of the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the enhanced signaling may be evidenced by enhanced proliferation of the cell. In some cases, the proliferation of the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the proliferation of the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the enhanced signaling may be evidenced by enhanced intracellular signaling of the cell. In some cases, the enhanced intracellular signaling may be evidenced by modification (e.g., structural modification or chemical modification) of the receptor and/or one or more signaling proteins of the receptor. Examples of the chemical modification may include dephosphorylation, phosphorylation, acetylation, methylation, ubiquitination, proteolytic processing, or combination thereof. In some cases, the chemical modification of the receptor and/or one or more signaling proteins of the receptor of the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the chemical modification of the receptor and/or one or more signaling proteins of the receptor of the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the enhanced intracellular signaling may be evidenced by expression of one or more target polynucleotides or polypeptides of the receptor. The one or more target polynucleotides or polypeptides of the receptor may be encoded by the gene of the cell. In some cases, the expression of the one or more target polynucleotides or polypeptides of the receptor in the cell may be increased by at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the expression of the one or more target polynucleotides or polypeptides of the receptor in the cell may be increased by at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the expression of the one or more target polynucleotides or polypeptides of the receptor in the cell may be decreased by at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the expression of the one or more target polynucleotides or polypeptides of the receptor in the cell may be decreased by at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the enhanced signaling may be evidenced by enhanced cytotoxicity against a target cell. In some cases, the target cell may be a diseased cell, a tumor cell, and/or a cancer cell. In some cases, the cytotoxicity of the cell against the target cell may be enhanced by at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the cytotoxicity of the cell against the target cell may be enhanced by at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the enhanced signaling may be evidenced by enhanced ability to reduce a size of or obliterate a tumor. In some cases, the enhanced signaling of the receptor may be measured during and/or subsequent to activation of the receptor. In some cases, upon activation of the receptor of the cell, the size of the tumor may be reduced by at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising receptor and the adaptor protein without the at least one heterologous NES. In some cases, upon activation of the receptor of the cell, the size of the tumor may be reduced by at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the receptor and the adaptor protein without the at least one heterologous NES. In some cases, upon activation of the receptor of the cell, obliteration of the tumor by the cell comprising the receptor and the chimeric polypeptide may occur faster by at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising receptor and the adaptor protein without the at least one heterologous NES. In some cases, upon activation of the receptor of the cell, obliteration of the tumor by the cell comprising the receptor and the chimeric polypeptide may occur faster by at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the receptor and the adaptor protein without the at least one heterologous NES.

In a different aspect, the present disclosure provides a method of increasing half-life of an adaptor protein of a receptor in a cell. In some cases, the method may comprise expressing a system in the cell. The system may be any one of the subject systems provided herein, such as, for example, any one of the subject receptors (e.g., endogenous or CAR) provided herein, any one of the subject chimeric polypeptides provided herein, and/or any one of the subject GMP provided herein. In some case, the system may comprise a chimeric polypeptide comprising the adaptor protein of the receptor linked to at least one nuclear export signal (NES) (e.g., at least one heterologous NES). In some cases, the increase in the half-life of the adaptor protein linked to the at least one heterologous NES, as compared to the adaptor protein without the at least one heterologous NES, may be evidenced by (i) increased amount of the adaptor protein that is membrane bound in the cell or (ii) higher steady state amount of the adaptor protein in the cell. In some cases, the increase in the half-life of the adaptor protein linked to the at least one heterologous NES, as compared to the adaptor protein without the at least one heterologous NES, may be evidenced by (i) increased amount of the adaptor protein that is membrane bound in the cell and (ii) higher steady state amount of the adaptor protein in the cell.

In some cases, the half-life of the adaptor protein may be measured prior to, during, or subsequent to activation of the receptor. In some cases, the half-life of the adaptor protein may be measured prior to, during, and subsequent to activation of the receptor.

In some cases, the increase in the half-life of the adaptor protein linked to the at least one heterologous NES may be evidenced by the increased amount of the adaptor protein (e.g., as part of the chimeric polypeptide) that is membrane bound in the cell. In some cases, the amount of the adaptor protein that is membrane bound in the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the amount of the adaptor protein that is membrane bound in the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

In some cases, the increase in the half-life of the adaptor protein linked to the at least one heterologous NES may be evidenced by the higher steady state amount of the adaptor protein in the cell. In some cases, the steady state amount of the adaptor protein in the cell may be at least about 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to a cell comprising the adaptor protein without the at least one heterologous NES. In some cases, the steady state amount of the adaptor protein in the cell may be at most about 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to a cell comprising the adaptor protein without the at least one heterologous NES.

### Receptor

In some cases, the receptor may be a chimeric receptor. In some cases, the ligand binding domain of the chimeric receptor may be heterologous to the cell. In some cases, the chimeric receptor may comprise a CAR. The CAR may comprise at least a portion of a Notch receptor, a G-protein coupled receptor (GPCR), an integrin receptor, a cadherin receptor, a receptor tyrosine kinase, a death receptor, an immune receptor. The immune receptor may comprise a T cell receptor (TCR). The TCR may comprise TCRA, TCRB, TCRG, and/or TCRD. The TCR may comprise a co-receptor of TCR, such as, CD3, CD4, and/or CD8. CD3 may comprise CD3E, CD3D, CD3G, and/or CD3Z. The CAR may comprise at least a portion of an intracellular portion of a TCR complex. As an alternative, the CAR may not comprise any portion of an intracellular portion of the TCR complex. The CAR may comprise one or more signaling capabilities of the TCR complex. As an alternative, the CAR may not comprise any signaling capability of the TCR complex.

Non-limiting examples of antigens which can be bound by a ligand interacting domain of a receptor or a CAR of a subject system can include, but are not limited to, 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β 3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP- ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFR_{V}III), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRCSD), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon a/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β1, TGF-β2, TGF-β3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and κ-light chain.

In some cases, the chimeric receptor of a subject system can comprise at least a portion of an endogenous receptor, or any derivative, variant or fragment thereof. The chimeric receptor can bind specifically to at least one antigen (e.g., at least one ligand), for example via an antigen interacting domain (also referred to herein as an “extracellular sensor domain”). The chimeric receptor can, in response to ligand binding, undergo a modification such as a conformational change and/or chemical modification. Such modification(s) can recruit to the chimeric receptor binding partners (e.g., partners such as proteins) including, but not limited to, signaling proteins involved in signaling events and various cellular processes. Signaling proteins, for example, can be involved in regulating (e.g., activating and/or de-activating) a cellular response such as programmed changes in gene expression via translational regulation; transcriptional regulation; and epigenetic modification including the regulation of methylation, acetylation, phosphorylation, ubiquitylation, sumoylation, ribosylation, and citrullination. Conformational changes of the chimeric receptor can expose one or more regions of the chimeric receptor which was previously not exposed, and the exposed region can recruit and/or bind signaling protein(s). Chemical modifications on a receptor, for example phosphorylation and/or dephosphorylation (e.g., at tyrosine, serine, threonine, and/or any other suitable amino acid residue), can also recruit signaling proteins involved in regulating intracellular processes. Signaling proteins can bind directly to a receptor or indirectly to a receptor, for example as part of a larger complex.

In some cases, the chimeric receptor polypeptide can comprise at least a portion of a transmembrane receptor. The transmembrane receptor may detect at least one signal (i.e., ligand), such as a small molecule, ion, or protein, from the surrounding environment (e.g., extracellular and/or intracellular environment) and can initiate a cellular response via at least one signaling cascade involving additional proteins and signaling molecules. The transmembrane receptor may translocate from one region of a cell to another, for example from the plasma membrane or cytoplasm to the nucleus and vice versa. Such translocation can be conditional upon ligand binding to the transmembrane receptor. Examples of the transmembrane receptor may include, but are not limited to, Notch receptors; G-protein coupled receptors (GPCRs); integrin receptors; cadherin receptors; catalytic receptors including receptors possessing enzymatic activity and receptors, which, rather than possessing intrinsic enzymatic activity, act by stimulating non-covalently associated enzymes (e.g., kinases); death receptors such as members of the tumor necrosis factor receptor (TNFR) superfamily; and immune receptors.

In some cases, the chimeric receptor polypeptide may comprise a Notch, or any derivative, variant or fragment thereof, selected from Notch1, Notch2, Notch3, and Notch4 or any homolog thereof.

In some cases, the chimeric receptor polypeptide may comprise a GPCR, or any derivative, variant or fragment thereof, selected from Class A Orphans; Class B Orphans; Class C Orphans; taste receptors, type 1; taste receptors, type 2; 5-hydroxytryptamine receptors; acetylcholine receptors (muscarinic); adenosine receptors; adhesion class GPCRs; adrenoceptors; angiotensin receptors; apelin receptor; bile acid receptor; bombesin receptors; bradykinin receptors; calcitonin receptors; calcium-sensing receptors; cannabinoid receptors; chemerin receptor; chemokine receptors; cholecystokinin receptors; class Frizzled GPCRs (e.g., Wnt receptors); complement peptide receptors; corticotropin-releasing factor receptors; dopamine receptors; endothelin receptors; G protein-coupled estrogen receptor; formylpeptide receptors; free fatty acid receptors; GABAB receptors; galanin receptors; ghrelin receptor; glucagon receptor family; glycoprotein hormone receptors; gonadotrophin-releasing hormone receptors; GPR18, GPR55 and GPR119; histamine receptors; hydroxycarboxylic acid receptors; kisspeptin receptor; leukotriene receptors; lysophospholipid (LPA) receptors; lysophospholipid (S1P) receptors; melanin-concentrating hormone receptors; melanocortin receptors; melatonin receptors; metabotropic glutamate receptors; motilin receptor; neuromedin U receptors; neuropeptide FF/neuropeptide AF receptors; neuropeptide S receptor; neuropeptide W/neuropeptide B receptors; neuropeptide Y receptors; neurotensin receptors; opioid receptors; orexin receptors; oxoglutarate receptor; P2Y receptors; parathyroid hormone receptors; platelet-activating factor receptor; prokineticin receptors; prolactin-releasing peptide receptor; prostanoid receptors; proteinase-activated receptors; QRFP receptor; relaxin family peptide receptors; somatostatin receptors; succinate receptor; tachykinin receptors; thyrotropin-releasing hormone receptors; trace amine receptor; urotensin receptor; vasopressin and oxytocin receptors; VIP and PACAP receptors.

In some cases, the chimeric receptor polypeptide may comprise a GPCR selected from the group consisting of: 5-hydroxytryptamine (serotonin) receptor 1A (HTR1A), 5-hydroxytryptamine (serotonin) receptor 1B (HTR1B), 5-hydroxytryptamine (serotonin) receptor 1D (HTR1D), 5-hydroxytryptamine (serotonin) receptor 1E (HTR1E), 5-hydroxytryptamine (serotonin) receptor 1F (HTR1F), 5-hydroxytryptamine (serotonin) receptor 2A (HTR2A), 5-hydroxytryptamine (serotonin) receptor 2B (HTR2B), 5-hydroxytryptamine (serotonin) receptor 2C (HTR2C), 5-hydroxytryptamine (serotonin) receptor 4 (HTR4), 5-hydroxytryptamine (serotonin) receptor 5A (HTR5A), 5-hydroxytryptamine (serotonin) receptor 5B (HTR5BP), 5-hydroxytryptamine (serotonin) receptor 6 (HTR6), 5-hydroxytryptamine (serotonin) receptor 7, adenylate cyclase-coupled (HTR7), cholinergic receptor, muscarinic 1 (CHRM1), cholinergic receptor, muscarinic 2 (CHRM2), cholinergic receptor, muscarinic 3 (CHRM3), cholinergic receptor, muscarinic 4 (CHRM4), cholinergic receptor, muscarinic 5 (CHRM5), adenosine A1 receptor (ADORA1), adenosine A2a receptor (ADORA2A), adenosine A2b receptor (ADORA2B), adenosine A3 receptor (ADORA3), adhesion G protein-coupled receptor A1 (ADGRA1), adhesion G protein-coupled receptor A2 (ADGRA2), adhesion G protein-coupled receptor A3 (ADGRA3), adhesion G protein-coupled receptor B1 (ADGRB 1), adhesion G protein-coupled receptor B2 (ADGRB2), adhesion G protein-coupled receptor B3 (ADGRB3), cadherin EGF LAG seven-pass G-type receptor 1 (CELSR1), cadherin EGF LAG seven-pass G-type receptor 2 (CELSR2), cadherin EGF LAG seven-pass G-type receptor 3 (CELSR3), adhesion G protein-coupled receptor D1 (ADGRD1), adhesion G protein-coupled receptor D2 (ADGRD2), adhesion G protein-coupled receptor E1 (ADGRE1), adhesion G protein-coupled receptor E2 (ADGRE2), adhesion G protein-coupled receptor E3 (ADGRE3), adhesion G protein-coupled receptor E4 (ADGRE4P), adhesion G protein-coupled receptor E5 (ADGRE5), adhesion G protein-coupled receptor F1 (ADGRF1), adhesion G protein-coupled receptor F2 (ADGRF2), adhesion G protein-coupled receptor F3 (ADGRF3), adhesion G protein-coupled receptor F4 (ADGRF4), adhesion G protein-coupled receptor F5 (ADGRF5), adhesion G protein-coupled receptor G1 (ADGRG1), adhesion G protein-coupled receptor G2 (ADGRG2), adhesion G protein-coupled receptor G3 (ADGRG3), adhesion G protein-coupled receptor G4 (ADGRG4), adhesion G protein-coupled receptor G5 (ADGRG5), adhesion G protein-coupled receptor G6 (ADGRG6), adhesion G protein-coupled receptor G7 (ADGRG7), adhesion G protein-coupled receptor L1 (ADGRL1), adhesion G protein-coupled receptor L2 (ADGRL2), adhesion G protein-coupled receptor L3 (ADGRL3), adhesion G protein-coupled receptor L4 (ADGRL4), adhesion G protein-coupled receptor V1 (ADGRV1), adrenoceptor alpha 1A (ADRA1A), adrenoceptor alpha 1B (ADRA1B), adrenoceptor alpha 1D (ADRA1D), adrenoceptor alpha 2A (ADRA2A), adrenoceptor alpha 2B (ADRA2B), adrenoceptor alpha 2C (ADRA2C), adrenoceptor beta 1 (ADRB1), adrenoceptor beta 2 (ADRB2), adrenoceptor beta 3 (ADRB3), angiotensin II receptor type 1 (AGTR1), angiotensin II receptor type 2 (AGTR2), apelin receptor (APLNR), G protein-coupled bile acid receptor 1 (GPBAR1), neuromedin B receptor (NMBR), gastrin releasing peptide receptor (GRPR), bombesin like receptor 3 (BRS3), bradykinin receptor B1 (BDKRB1), bradykinin receptor B2 (BDKRB2), calcitonin receptor (CALCR), calcitonin receptor like receptor (CALCRL), calcium sensing receptor (CASR), G protein-coupled receptor, class C (GPRC6A), cannabinoid receptor 1 (brain) (CNR1), cannabinoid receptor 2 (CNR2), chemerin chemokine-like receptor 1 (CMKLR1), chemokine (C-C motif) receptor 1 (CCR1), chemokine (C-C motif) receptor 2 (CCR2), chemokine (C-C motif) receptor 3 (CCR3), chemokine (C-C motif) receptor 4 (CCR4), chemokine (C-C motif) receptor 5 (gene/pseudogene) (CCRS), chemokine (C-C motif) receptor 6 (CCR6), chemokine (C-C motif) receptor 7 (CCR7), chemokine (C-C motif) receptor 8 (CCR8), chemokine (C-C motif) receptor 9 (CCR9), chemokine (C-C motif) receptor 10 (CCR10), chemokine (C-X-C motif) receptor 1 (CXCR1), chemokine (C-X-C motif) receptor 2 (CXCR2), chemokine (C-X-C motif) receptor 3 (CXCR3), chemokine (C-X-C motif) receptor 4 (CXCR4), chemokine (C-X-C motif) receptor 5 (CXCR5), chemokine (C-X-C motif) receptor 6 (CXCR6), chemokine (C-X3-C motif) receptor 1 (CX3CR1), chemokine (C motif) receptor 1 (XCR1), atypical chemokine receptor 1 (Duffy blood group) (ACKR1), atypical chemokine receptor 2 (ACKR2), atypical chemokine receptor 3 (ACKR3), atypical chemokine receptor 4 (ACKR4), chemokine (C-C motif) receptor-like 2 (CCRL2), cholecystokinin A receptor (CCKAR), cholecystokinin B receptor (CCKBR), G protein-coupled receptor 1 (GPR1), bombesin like receptor 3 (BRS3), G protein-coupled receptor 3 (GPR3), G protein-coupled receptor 4 (GPR4), G protein-coupled receptor 6 (GPR6), G protein-coupled receptor 12 (GPR12), G protein-coupled receptor 15 (GPR15), G protein-coupled receptor 17 (GPR17), G protein-coupled receptor 18 (GPR18), G protein-coupled receptor 19 (GPR19), G protein-coupled receptor 20 (GPR20), G protein-coupled receptor 21 (GPR21), G protein-coupled receptor 22 (GPR22), G protein-coupled receptor 25 (GPR25), G protein-coupled receptor 26 (GPR26), G protein-coupled receptor 27 (GPR27), G protein-coupled receptor 31 (GPR31), G protein-coupled receptor 32 (GPR32), G protein-coupled receptor 33 (gene/pseudogene) (GPR33), G protein-coupled receptor 34 (GPR34), G protein-coupled receptor 35 (GPR35), G protein-coupled receptor 37 (endothelin receptor type B-like) (GPR37), G protein-coupled receptor 37 like 1 (GPR37L1), G protein-coupled receptor 39 (GPR39), G protein-coupled receptor 42 (gene/pseudogene) (GPR42), G protein-coupled receptor 45 (GPR45), G protein-coupled receptor 50 (GPR50), G protein-coupled receptor 52 (GPR52), G protein-coupled receptor 55 (GPR55), G protein-coupled receptor 61 (GPR61), G protein-coupled receptor 62 (GPR62), G protein-coupled receptor 63 (GPR63), G protein-coupled receptor 65 (GPR65), G protein-coupled receptor 68 (GPR68), G protein-coupled receptor 75 (GPR75), G protein-coupled receptor 78 (GPR78), G protein-coupled receptor 79 (GPR79), G protein-coupled receptor 82 (GPR82), G protein-coupled receptor 83 (GPR83), G protein-coupled receptor 84 (GPR84), G protein-coupled receptor 85 (GPR85), G protein-coupled receptor 87 (GPR87), G protein-coupled receptor 88 (GPR88), G protein-coupled receptor 101 (GPR101), G protein-coupled receptor 119 (GPR119), G protein-coupled receptor 132 (GPR132), G protein-coupled receptor 135 (GPR135), G protein-coupled receptor 139 (GPR139), G protein-coupled receptor 141 (GPR141), G protein-coupled receptor 142 (GPR142), G protein-coupled receptor 146 (GPR146), G protein-coupled receptor 148 (GPR148), G protein-coupled receptor 149 (GPR149), G protein-coupled receptor 150 (GPR150), G protein-coupled receptor 151 (GPR151), G protein-coupled receptor 152 (GPR152), G protein-coupled receptor 153 (GPR153), G protein-coupled receptor 160 (GPR160), G protein-coupled receptor 161 (GPR161), G protein-coupled receptor 162 (GPR162), G protein-coupled receptor 171 (GPR171), G protein-coupled receptor 173 (GPR173), G protein-coupled receptor 174 (GPR174), G protein-coupled receptor 176 (GPR176), G protein-coupled receptor 182 (GPR182), G protein-coupled receptor 183 (GPR183), leucine-rich repeat containing G protein-coupled receptor 4 (LGR4), leucine-rich repeat containing G protein-coupled receptor 5 (LGR5), leucine-rich repeat containing G protein-coupled receptor 6 (LGR6), MASI proto-oncogene (MAS 1), MASI proto-oncogene like (MAS1L), MAS related GPR family member D (MRGPRD), MAS related GPR family member E (MRGPRE), MAS related GPR family member F (MRGPRF), MAS related GPR family member G (MRGPRG), MAS related GPR family member X1 (MRGPRX1), MAS related GPR family member X2 (MRGPRX2), MAS related GPR family member X3 (MRGPRX3), MAS related GPR family member X4 (MRGPRX4), opsin 3 (OPN3), opsin 4 (OPN4), opsin 5 (OPNS), purinergic receptor P2Y (P2RY8), purinergic receptor P2Y (P2RY10), trace amine associated receptor 2 (TAAR2), trace amine associated receptor 3 (gene/pseudogene) (TAAR3), trace amine associated receptor 4 (TAAR4P), trace amine associated receptor 5 (TAAR5), trace amine associated receptor 6 (TAAR6), trace amine associated receptor 8 (TAAR8), trace amine associated receptor 9 (gene/pseudogene) (TAAR9), G protein-coupled receptor 156 (GPR156), G protein-coupled receptor 158 (GPR158), G protein-coupled receptor 179 (GPR179), G protein-coupled receptor, class C (GPRC5A), G protein-coupled receptor, class C (GPRC5B), G protein-coupled receptor, class C (GPRC5C), G protein-coupled receptor, class C (GPRC5D), frizzled class receptor 1 (FZD1), frizzled class receptor 2 (FZD2), frizzled class receptor 3 (FZD3), frizzled class receptor 4 (FZD4), frizzled class receptor 5 (FZD5), frizzled class receptor 6 (FZD6), frizzled class receptor 7 (FZD7), frizzled class receptor 8 (FZD8), frizzled class receptor 9 (FZD9), frizzled class receptor 10 (FZD10), smoothened, frizzled class receptor (SMO), complement component 3a receptor 1 (C3AR1), complement component 5a receptor 1 (C5AR1), complement component 5a receptor 2 (C5AR2), corticotropin releasing hormone receptor 1 (CRHR1), corticotropin releasing hormone receptor 2 (CRHR2), dopamine receptor D1 (DRD1), dopamine receptor D2 (DRD2), dopamine receptor D3 (DRD3), dopamine receptor D4 (DRD4), dopamine receptor D5 (DRD5), endothelin receptor type A (EDNRA), endothelin receptor type B (EDNRB), formyl peptide receptor 1 (FPR1), formyl peptide receptor 2 (FPR2), formyl peptide receptor 3 (FPR3), free fatty acid receptor 1 (FFAR1), free fatty acid receptor 2 (FFAR2), free fatty acid receptor 3 (FFAR3), free fatty acid receptor 4 (FFAR4), G protein-coupled receptor 42 (gene/pseudogene) (GPR42), gamma-aminobutyric acid (GABA) B receptor, 1 (GABBR1), gamma-aminobutyric acid (GABA) B receptor, 2 (GABBR2), galanin receptor 1 (GALR1), galanin receptor 2 (GALR2), galanin receptor 3 (GALR3), growth hormone secretagogue receptor (GHSR), growth hormone releasing hormone receptor (GHRHR), gastric inhibitory polypeptide receptor (GIPR), glucagon like peptide 1 receptor (GLP1R), glucagon-like peptide 2 receptor (GLP2R), glucagon receptor (GCGR), secretin receptor (SCTR), follicle stimulating hormone receptor (FSHR), luteinizing hormone/choriogonadotropin receptor (LHCGR), thyroid stimulating hormone receptor (TSHR), gonadotropin releasing hormone receptor (GNRHR), gonadotropin releasing hormone receptor 2 (pseudogene) (GNRHR2), G protein-coupled receptor 18 (GPR18), G protein-coupled receptor 55 (GPR55), G protein-coupled receptor 119 (GPR119), G protein-coupled estrogen receptor 1 (GPER1), histamine receptor H1 (HRH1), histamine receptor H2 (HRH2), histamine receptor H3 (HRH3), histamine receptor H4 (HRH4), hydroxycarboxylic acid receptor 1 (HCAR1), hydroxycarboxylic acid receptor 2 (HCAR2), hydroxycarboxylic acid receptor 3 (HCAR3), KISS1 receptor (KISS1R), leukotriene B4 receptor (LTB4R), leukotriene B4 receptor 2 (LTB4R2), cysteinyl leukotriene receptor 1 (CYSLTR1), cysteinyl leukotriene receptor 2 (CYSLTR2), oxoeicosanoid (OXE) receptor 1 (OXER1), formyl peptide receptor 2 (FPR2), lysophosphatidic acid receptor 1 (LPAR1), lysophosphatidic acid receptor 2 (LPAR2), lysophosphatidic acid receptor 3 (LPAR3), lysophosphatidic acid receptor 4 (LPAR4), lysophosphatidic acid receptor 5 (LPAR5), lysophosphatidic acid receptor 6 (LPAR6), sphingosine-1-phosphate receptor 1 (S1PR1), sphingosine-1-phosphate receptor 2 (S1PR2), sphingosine-1-phosphate receptor 3 (S1PR3), sphingosine-1-phosphate receptor 4 (S1PR4), sphingosine-1-phosphate receptor 5 (S1PR5), melanin concentrating hormone receptor 1 (MCHR1), melanin concentrating hormone receptor 2 (MCHR2), melanocortin 1 receptor (alpha melanocyte stimulating hormone receptor) (MC1R), melanocortin 2 receptor (adrenocorticotropic hormone) (MC2R), melanocortin 3 receptor (MC3R), melanocortin 4 receptor (MC4R), melanocortin 5 receptor (MC5R), melatonin receptor 1A (MTNR1A), melatonin receptor 1B (MTNR1B), glutamate receptor, metabotropic 1 (GRM1), glutamate receptor, metabotropic 2 (GRM2), glutamate receptor, metabotropic 3 (GRM3), glutamate receptor, metabotropic 4 (GRM4), glutamate receptor, metabotropic 5 (GRM5), glutamate receptor, metabotropic 6 (GRM6), glutamate receptor, metabotropic 7 (GRM7), glutamate receptor, metabotropic 8 (GRM8), motilin receptor (MLNR), neuromedin U receptor 1 (NMUR1), neuromedin U receptor 2 (NMUR2), neuropeptide FF receptor 1 (NPFFR1), neuropeptide FF receptor 2 (NPFFR2), neuropeptide S receptor 1 (NPSR1), neuropeptides B/W receptor 1 (NPBWR1), neuropeptides B/W receptor 2 (NPBWR2), neuropeptide Y receptor Y1 (NPY1R), neuropeptide Y receptor Y2 (NPY2R), neuropeptide Y receptor Y4 (NPY4R), neuropeptide Y receptor Y5 (NPY5R), neuropeptide Y receptor Y6 (pseudogene) (NPY6R), neurotensin receptor 1 (high affinity) (NTSR1), neurotensin receptor 2 (NTSR2), opioid receptor, delta 1 (OPRD1), opioid receptor, kappa 1 (OPRK1), opioid receptor, mu 1 (OPRM1), opiate receptor-like 1 (OPRL1), hypocretin (orexin) receptor 1 (HCRTR1), hypocretin (orexin) receptor 2 (HCRTR2), G protein-coupled receptor 107 (GPR107), G protein-coupled receptor 137 (GPR137), olfactory receptor family 51 subfamily E member 1 (OR51E1), transmembrane protein, adipocyte associated 1 (TPRA1), G protein-coupled receptor 143 (GPR143), G protein-coupled receptor 157 (GPR157), oxoglutarate (alpha-ketoglutarate) receptor 1 (OXGR1), purinergic receptor P2Y (P2RY1), purinergic receptor P2Y (P2RY2), pyrimidinergic receptor P2Y (P2RY4), pyrimidinergic receptor P2Y (P2RY6), purinergic receptor P2Y (P2RY11), purinergic receptor P2Y (P2RY12), purinergic receptor P2Y (P2RY13), purinergic receptor P2Y (P2RY14), parathyroid hormone 1 receptor (PTH1R), parathyroid hormone 2 receptor (PTH2R), platelet-activating factor receptor (PTAFR), prokineticin receptor 1 (PROKR1), prokineticin receptor 2 (PROKR2), prolactin releasing hormone receptor (PRLHR), prostaglandin D2 receptor (DP) (PTGDR), prostaglandin D2 receptor 2 (PTGDR2), prostaglandin E receptor 1 (PTGER1), prostaglandin E receptor 2 (PTGER2), prostaglandin E receptor 3 (PTGER3), prostaglandin E receptor 4 (PTGER4), prostaglandin F receptor (PTGFR), prostaglandin 12 (prostacyclin) receptor (IP) (PTGIR), thromboxane A2 receptor (TBXA2R), coagulation factor II thrombin receptor (F2R), F2R like trypsin receptor 1 (F2RL1), coagulation factor II thrombin receptor like 2 (F2RL2), F2R like thrombin/trypsin receptor 3 (F2RL3), pyroglutamylated RFamide peptide receptor (QRFPR), relaxin/insulin-like family peptide receptor 1 (RXFP1), relaxin/insulin-like family peptide receptor 2 (RXFP2), relaxin/insulin-like family peptide receptor 3 (RXFP3), relaxin/insulin-like family peptide receptor 4 (RXFP4), somatostatin receptor 1 (SSTR1), somatostatin receptor 2 (SSTR2), somatostatin receptor 3 (SSTR3), somatostatin receptor 4 (SSTR4), somatostatin receptor 5 (SSTR5), succinate receptor 1 (SUCNR1), tachykinin receptor 1 (TACR1), tachykinin receptor 2 (TACR2), tachykinin receptor 3 (TACR3), taste 1 receptor member 1 (TAS1R1), taste 1 receptor member 2 (TAS1R2), taste 1 receptor member 3 (TAS1R3), taste 2 receptor member 1 (TAS2R1), taste 2 receptor member 3 (TAS2R3), taste 2 receptor member 4 (TAS2R4), taste 2 receptor member 5 (TAS2R5), taste 2 receptor member 7 (TAS2R7), taste 2 receptor member 8 (TAS2R8), taste 2 receptor member 9 (TAS2R9), taste 2 receptor member 10 (TAS2R10), taste 2 receptor member 13 (TAS2R13), taste 2 receptor member 14 (TAS2R14), taste 2 receptor member 16 (TAS2R16), taste 2 receptor member 19 (TAS2R19), taste 2 receptor member 20 (TAS2R20), taste 2 receptor member 30 (TAS2R30), taste 2 receptor member 31 (TAS2R31), taste 2 receptor member 38 (TAS2R38), taste 2 receptor member 39 (TAS2R39), taste 2 receptor member 40 (TAS2R40), taste 2 receptor member 41 (TAS2R41), taste 2 receptor member 42 (TAS2R42), taste 2 receptor member 43 (TAS2R43), taste 2 receptor member 45 (TAS2R45), taste 2 receptor member 46 (TAS2R46), taste 2 receptor member 50 (TAS2R50), taste 2 receptor member 60 (TAS2R60), thyrotropin-releasing hormone receptor (TRHR), trace amine associated receptor 1 (TAAR1), urotensin 2 receptor (UTS2R), arginine vasopressin receptor 1A (AVPR1A), arginine vasopressin receptor 1B (AVPR1B), arginine vasopressin receptor 2 (AVPR2), oxytocin receptor (OXTR), adenylate cyclase activating polypeptide 1 (pituitary) receptor type I (ADCYAP1R1), vasoactive intestinal peptide receptor 1 (VIPR1), vasoactive intestinal peptide receptor 2 (VIPR2), any derivative thereof, any variant thereof, and any fragment thereof.

The chimeric receptor comprising a GPCR, or any derivative, variant or fragment thereof, may bind an antigen comprising any suitable GPCR ligand, or any derivative, variant or fragment thereof. Non-limiting examples of ligands which can be bound by a GPCR include (―)-adrenaline, (-)-noradrenaline, (lyso)phospholipid mediators, [des-Arg10]kallidin, [des-Arg9]bradykinin, [des-Gln14]ghrelin, [Hyp3]bradykinin, [Leu]enkephalin, [Met]enkephalin, 12-hydroxyheptadecatrienoic acid, 12R-HETE, 12S-HETE, 12S-HPETE, 15S-HETE, 17β-estradiol, 20-hydroxy-LTB4, 2-arachidonoylglycerol, 2-oleoyl-LPA, 3-hydroxyoctanoic acid, 5-hydroxytryptamine, 5-oxo-15-HETE, 5-oxo-ETE, 5-oxo-ETrE, 5-oxo-ODE, 5S-HETE, 5S-HPETE, 7α,25-dihydroxycholesterol, acetylcholine, ACTH, adenosine diphosphate, adenosine, adrenomedullin 2/intermedin, adrenomedullin, amylin, anandamide, angiotensin II, angiotensin III, annexin I, apelin receptor early endogenous ligand, apelin-13, apelin-17, apelin-36, aspirin triggered lipoxin A4, aspirin-triggered resolvin D1, ATP, beta-defensin 4A, big dynorphin, bovine adrenal medulla peptide 8-22, bradykinin, C3a, C5a, Ca2+, calcitonin gene related peptide, calcitonin, cathepsin G, CCK-33, CCK-4, CCK-8, CCL1, CCL11, CCL13, CCL14, CCL15, CCL16, CCL17, CCL19, CCL2, CCL20, CCL21, CCL22, CCL23, CCL24, CCL25, CCL26, CCL27, CCL28, CCL3, CCL4, CCL5, CCL7, CCL8, chemerin, chenodeoxycholic acid, cholic acid, corticotrophin-releasing hormone, CST-17, CX3CL1, CXCL1, CXCL10, CXCL11, CXCL12a, CXCL12β, CXCL13, CXCL16, CXCL2, CXCL3, CXCL5, CXCL6, CXCL7, CXCL8, CXCL9, cysteinyl-leukotrienes (CysLTs), uracil nucleotides, deoxycholic acid, dihydrosphingosine-1-phosphate, dioleoylphosphatidic acid, dopamine, dynorphin A, dynorphin A-(1-13), dynorphin A-(1-8), dynorphin B, endomorphin-1, endothelin-1, endothelin-2, endothelin-3, F2L, Free fatty acids, FSH, GABA, galanin, galanin-like peptide, gastric inhibitory polypeptide, gastrin-17, gastrin-releasing peptide, ghrelin, GHRH, glucagon, glucagon-like peptide 1-(7-36) amide, glucagon-like peptide 1-(7-37), glucagon-like peptide 2, glucagon-like peptide 2-(3-33), GnRH I, GnRH II, GRP-(18-27), hCG, histamine, humanin, INSL3, INSL5, kallidin, kisspeptin-10, kisspeptin-13, kisspeptin-14, kisspeptin-54, kynurenic acid, large neuromedin N, large neurotensin, L-glutamic acid, LH, lithocholic acid, L-lactic acid, long chain carboxylic acids, LPA, LTB4, LTC4, LTD4, LTE4, LXA4, Lys-[Hyp3]-bradykinin, lysophosphatidylinositol, lysophosphatidylserine, Medium-chain-length fatty acids, melanin-concentrating hormone, melatonin, methylcarbamyl PAF, Mg2+, motilin, N-arachidonoylglycine, neurokinin A, neurokinin B, neuromedin B, neuromedin N, neuromedin S-33, neuromedin U-25, neuronostatin, neuropeptide AF, neuropeptide B-23, neuropeptide B-29, neuropeptide FF, neuropeptide S, neuropeptide SF, neuropeptide W-23, neuropeptide W-30, neuropeptide Y, neuropeptide Y-(3-36), neurotensin, nociceptin/orphanin FQ, N-oleoylethanolamide, obestatin, octopamine, orexin-A, orexin-B, Oxysterols, oxytocin, PACAP-27, PACAP-38, PAF, pancreatic polypeptide, peptide YY, PGD2, PGE2, PGF2α, PGI2, PGJ2, PHM, phosphatidylserine, PHV, prokineticin-1, prokineticin-2, prokineticin-2β, prosaposin, PrRP-20, PrRP-31, PTH, PTHrP, PTHrP-(1-36), QRFP43, relaxin, relaxin-1, relaxin-3, resolvin D1, resolvin E1, RFRP-1, RFRP-3, R-spondins, secretin, serine proteases, sphingosine 1-phosphate, sphingosylphosphorylcholine, SRIF-14, SRIF-28, substance P, succinic acid, thrombin, thromboxane A2, TIP39, T-kinin, TRH, TSH, tyramine, UDP-glucose, uridine diphosphate, urocortin 1, urocortin 2, urocortin 3, urotensin II-related peptide, urotensin-II, vasopressin, VIP, Wnt, Wnt-1, Wnt-10a, Wnt-10b, Wnt-11, Wnt-16, Wnt-2, Wnt-2b, Wnt-3, Wnt-3a, Wnt-4, Wnt-5a, Wnt-5b, Wnt-6, Wnt-7a, Wnt-7b, Wnt-8a, Wnt-8b, Wnt-9a, Wnt-9b, XCL1, XCL2, Zn2+, α-CGRP, α-ketoglutaric acid, α-MSH, α-neoendorphin, β-alanine, β-CGRP, β-D-hydroxybutyric acid, β-endorphin, β-MSH, β-neoendorphin, β-phenylethylamine, and γ-MSH.

In some cases, the chimeric receptor may comprise an integrin receptor a subunit, or any derivative, variant or fragment thereof, selected from the group consisting of: αl, α2, α3, α4, α5, α6, α7, α8, α9, α10, α11, αV, αL, αM, αX, αD, αE, and αIIb. In some embodiments, a chimeric receptor polypeptide comprises an integrin receptor β subunit, or any derivative, variant or fragment thereof, selected from the group consisting of: β1, β2, β3, β4, β5, β6, β7, and β8. Chimeric receptor polypeptides comprising an α subunit, a β subunit, or any derivative, variant or fragment thereof, can heterodimerize (e.g., α subunit dimerizing with a β subunit) to form an integrin receptor, or any derivative, variant or fragment thereof. Non-limiting examples of integrin receptors include an α1β1, α2β1, α3β1, α4β1, α5β1, α6β1, α7β1, α8β1, α9β1, α10β1, αVβ1, αLβ1, αMβ1, αXβ1, αDβ1, αIIbβ1, αEβ1, α1β2, α2β2, α3β2, α4β2, α5β2, α6β2, α7β2, α8β2, α9β2, α10β2, αVβ2, αLβ2, αMβ2, αXβ2, αDβ2, αIIbβ2, αEβ2, α1β3, α2β3, α3β3, α4β3, α5β3, α6β3, α7β3, α8β3, α9β3, α10β3, αVβ3, αLβ3, αMβ3, αXβ3, αDβ3, αIIbβ3, αEβ3, αlβ4, α2β4, α3β4, α4β4, α5β4, α6β4, α7β4, α8β4, α9β4, α10β4, αVβ4, αLβ4, αMβ4, αXβ4, αDβ4, αIIbβ4, αEβ4, α1β5, α2β5, α3β5, α4β5, α5β5, α6β5, α7β5, α8β5, α9β5, α10β5, αVβ5, αLβ5, αMβ5, αXβ5, αDβ5, αIIbβ5, αEβ5, α1β6, α2β6, α3β6, α4β6, α5β6, α6β6, α7β6, α8β6, α9β6, α10β6, αVβ6, αLβ6, αMβ6, αXβ6, αDβ6, αIIbβ6, αEβ6, α1β7, α2β7, α3β7, α4β7, α5β7, α6β7, α7β7, α8β7, α9β7, α10β7, αVβ7, αLβ7, αMβ7, αXβ7, αDβ7, αIIbβ7, αEβ7, α1β8, α2β8, α3β8, α4β8, α5β8, α6β8, α7β8, α8β8, α9β8, α10β8, αVβ8, αLβ8, αMβ8, αXβ8, αDβ8, αIIbβ8, and αEβ8 receptor. The chimeric receptor comprising an integrin subunit, or any derivative, variant or fragment thereof, may dimerize with an endogenous integrin subunit (e.g., wild-type integrin subunit).

In some cases, the chimeric receptor may comprise an integrin subunit, or any derivative, variant or fragment thereof, can bind an antigen comprising any suitable integrin ligand, or any derivative, variant or fragment thereof. Non-limiting examples of ligands which can be bound by an integrin receptor may include adenovirus penton base protein, beta-glucan, bone sialoprotein (BSP), Borrelia burgdorferi, Candida albicans, collagens (CN, e.g., CNI-IV), cytotactin/tenascin-C, decorsin, denatured collagen, disintegrins, E-cadherin, echovirus 1 receptor, epiligrin, Factor X, Fc epsilon RII (CD23), fibrin (Fb), fibrinogen (Fg), fibronectin (Fn), heparin, HIV Tat protein, iC3b, intercellular adhesion molecule (e.g., ICAM-1,2,3,4,5), invasin, L1 cell adhesion molecule (L1-CAM), laminin, lipopolysaccharide (LPS), MAdCAM-1, matrix metalloproteinase-2 (MMPe), neutrophil inhibitory factor (NIF), osteopontin (OP or OPN), plasminogen, prothrombin, sperm fertilin, thrombospondin (TSP), vascular cell adhesion molecule 1 (VCAM-1), vitronectin (VN or VTN), and von Willebrand factor (vWF).

In some cases, the chimeric receptor can comprise a cadherin, or any derivative, variant or fragment thereof, selected from a classical cadherin, a desmosoma cadherin, a protocadherin, and an unconventional cadherin. In some embodiments, a chimeric receptor polypeptide comprises a classical cadherin, or any derivative, variant or fragment thereof, selected from CDH1 (E-cadherin, epithelial), CDH2 (N-cadherin, neural), CDH12 (cadherin 12, type 2, N-cadherin 2), and CDH3 (P-cadherin, placental). In some embodiments, a chimeric receptor polypeptide comprises a desmosoma cadherin, or any derivative, variant or fragment thereof, selected from desmoglein (DSG1, DSG2, DSG3, DSG4) and desmocollin (DSC1, DSC2, DSC3). In some embodiments, a chimeric receptor polypeptide comprises a protocadherin, or any derivative, variant or fragment thereof, selected from PCDH1, PCDH10, PCDH11X, PCDH11Y, PCDH12, PCDH15, PCDH17, PCDH18, PCDH19, PCDH20, PCDH7, PCDH8, PCDH9, PCDHA1, PCDHA10, PCDHA11, PCDHA12, PCDHA13, PCDHA2, PCDHA3, PCDHA4, PCDHA5, PCDHA6, PCDHA7, PCDHA8, PCDHA9, PCDHAC1, PCDHAC2, PCDHB1, PCDHB10, PCDHB11, PCDHB12, PCDHB13, PCDHB14, PCDHB15, PCDHB 16, PCDHB 17, PCDHB 18, PCDHB2, PCDHB3, PCDHB4, PCDHB5, PCDHB6, PCDHB7, PCDHB8, PCDHB9, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, FAT, FAT2, and FAT). In some embodiments, a chimeric receptor polypeptide comprises an unconventional cadherin selected from CDH4 (R-cadherin, retinal), CDH5 (VE-cadherin, vascular endothelial), CDH6 (K-cadherin, kidney), CDH7 (cadherin 7, type 2), CDH8 (cadherin 8, type 2), CDH9 (cadherin 9, type 2, T1-cadherin), CDH10 (cadherin 10, type 2, T2-cadherin), CDH11 (OB-cadherin, osteoblast), CDH13 (T-cadherin, H-cadherin, heart), CDH15 (M-cadherin, myotubule), CDH16 (KSP-cadherin), CDH17 (LI cadherin, liver-intestine), CDH18 (cadherin 18, type 2), CDH19 (cadherin 19, type 2), CDH20 (cadherin 20, type 2), CDH23 (cadherin 23, neurosensory epithelium), CDH24, CDH26, CDH28, CELSR1, CELSR2, CELSR3, CLSTN1, CLSTN2, CLSTN3, DCHS1, DCHS2, LOC389118, PCLKC, RESDA1, and RET.

In some cases, the chimeric receptor may comprise at least an extracellular region (e.g., ligand binding domain) of a catalytic receptor such as a RTK, or any derivative, variant or fragment thereof. The chimeric receptor may comprise at least a membrane spanning region of a catalytic receptor such as a RTK, or any derivative, variant or fragment thereof. The chimeric receptor may comprise at least an intracellular region (e.g., cytosolic domain) of a catalytic receptor such as a RTK, or any derivative, variant or fragment thereof. The chimeric receptor may comprise an RTK, or any derivative, variant or fragment thereof, can recruit a binding partner. In some cases, ligand binding to a chimeric receptor comprising an RTK, or any derivative, variant or fragment thereof, results in a conformational change, chemical modification, or combination thereof, which recruits a binding partner to the chimeric receptor.

In some cases, the chimeric receptor may comprise a class IRTK (e.g., the epidermal growth factor (EGF) receptor family including EGFR; the ErbB family including ErbB-2, ErbB-3, and ErbB-4), a class II RTK (e.g., the insulin receptor family including INSR, IGF-1R, and IRR), a class III RTK (e.g., the platelet-derived growth factor (PDGF) receptor family including PDGFR-α, PDGFR-β, CSF-1R, KIT/SCFR, and FLK2/FLT3), a class IV RTK (e.g., the fibroblast growth factor (FGF) receptor family including FGFR-1, FGFR-2, FGFR-3, and FGFR-4), a class V RTK (e.g., the vascular endothelial growth factor (VEGF) receptor family including VEGFR1, VEGFR2, and VEGFR3), a class VIRTK (e.g., the hepatocyte growth factor (HGF) receptor family including hepatocyte growth factor receptor (HGFR/MET) and RON), a class VII RTK (e.g., the tropomyosin receptor kinase (Trk) receptor family including TRKA, TRKB, and TRKC), a class VIII RTK (e.g., the ephrin (Eph) receptor family including EPHA1, EPHA2, EPHA3, EPHA4, EPHA5, EPHA6, EPHA7, EPHA8, EPHB1, EPHB2, EPHB3, EPHB4, EPHB5, and EPHB6), a class IX RTK (e.g., AXL receptor family such as AXL, MER, and TRYO3), a class X RTK (e.g., LTK receptor family such as LTK and ALK), a class XI RTK (e.g., TIE receptor family such as TIE and TEK), a class XII RTK (e.g., ROR receptor family ROR1 and ROR2), a class XIII RTK (e.g., the discoidin domain receptor (DDR) family such as DDR1 and DDR2), a class XIV RTK (e.g., RET receptor family such as RET), a class XV RTK (e.g., KLG receptor family including PTK7), a class XVI RTK (e.g., RYK receptor family including Ryk), a class XVII RTK (e.g., MuSK receptor family such as MuSK), or any derivative, variant or fragment thereof.

The chimeric receptor comprising a RTK, or any derivative, variant or fragment thereof, may bind an antigen comprising any suitable RTK ligand, or any derivative, variant or fragment thereof. Non limiting examples of RTK ligands include growth factors, cytokines, and hormones. Growth factors include, for example, members of the epidermal growth factor family (e.g., epidermal growth factor or EGF, heparin-binding EGF-like growth factor or HB-EGF, transforming growth factor-α or TGF-α, amphiregulin or AR, epiregulin or EPR, epigen, betacellulin or BTC, neuregulin-1 or NRG1, neuregulin-2 or NRG2, neuregulin-3 or NRG3, and neuregulin-4 or NRG4), the fibroblast growth factor family (e.g., FGF1, FGF2, FGF3, FGF4, FGF5, FGF6, FGF7, FGF8, FGF9, FGF10, FGF11, FGF12, FGF13, FGF14, FGF15/19, FGF16, FGF17, FGF18, FGF20, FGF21, and FGF23), the vascular endothelial growth factor family (e.g., VEGF-A, VEGF-B, VEGF-C, VEGF-D, and PIGF), and the platelet-derived growth factor family (e.g., PDGFA, PDGFB, PDGFC, and PDGFD). Hormones include, for example, members of the insulin/IGF/relaxin family (e.g., insulin, insulin-like growth factors, relaxin family peptides including relaxin1, relaxin2, relaxin3, Leydig cell-specific insulin-like peptide (gene INSL3), early placenta insulin-like peptide (ELIP) (gene INSL4), insulin-like peptide 5 (gene INSL5), and insulin-like peptide 6).

In some cases, the chimeric receptor may comprise at least an extracellular region (e.g., ligand binding domain) of a catalytic receptor such as an RTSK, or any derivative, variant or fragment thereof. The chimeric receptor may comprise at least a membrane spanning region of a catalytic receptor such as an RTSK, or any derivative, variant or fragment thereof. The chimeric receptor may comprise at least an intracellular region (e.g., cytosolic domain) of a catalytic receptor such as an RTSK, or any derivative, variant or fragment thereof. The chimeric receptor polypeptide comprising an RTSK, or any derivative, variant or fragment thereof, may recruit a binding partner. In some cases, ligand binding to the chimeric receptor comprising an RTSK, or any derivative, variant or fragment thereof, may result in a conformational change, chemical modification, or combination thereof, which recruits a binding partner to the chimeric receptor.

The chimeric receptor comprising an RTSK, or any derivative, variant or fragment thereof, may phosphorylate a substrate at serine and/or threonine residues, and may select specific residues based on a consensus sequence. The chimeric receptor may comprise a type I RTSK, type II RTSK, or any derivative, variant or fragment thereof. The chimeric receptor comprising a type I receptor serine/threonine kinase may be inactive unless complexed with a type II receptor. In some cases, the chimeric receptor comprising a type II receptor serine/threonine may comprise a constitutively active kinase domain that can phosphorylate and activate a type I receptor when complexed with the type I receptor. A type II receptor serine/threonine kinase can phosphorylate the kinase domain of the type I partner, causing displacement of protein partners. Displacement of protein partners can allow binding and phosphorylation of other proteins, for example certain members of the SMAD family. The chimeric receptor can comprise a type I receptor, or any derivative, variant or fragment thereof, selected from the group consisting of: ALK1 (ACVRL1), ALK2 (ACVR1A), ALK3 (BMPR1A), ALK4 (ACVR1B), ALK5 (TGFβR1), ALK6 (BMPR1B), and ALK7 (ACVR1C). The chimeric receptor can comprise a type II receptor, or any derivative, variant or fragment thereof, selected from the group consisting of: TGFβR2, BMPR2, ACVR2A, ACVR2B, and AMHR2 (AMHR). The chimeric receptor can comprise a TGF-βreceptor, or any derivative, variant or fragment thereof.

In some cases, the chimeric receptor can comprise a receptor which stimulates non-covalently associated intracellular kinases, such as a Src kinase (e.g., c-Src, Yes, Fyn, Fgr, Lck, Hck, Blk, Lyn, and Frk) or a JAK kinase (e.g., JAK1, JAK2, JAK3, and TYK2) rather than possessing intrinsic enzymatic activity, or any derivative, variant or fragment thereof. These include the cytokine receptor superfamily such as receptors for cytokines and polypeptide hormones. Cytokine receptors generally contain an N-terminal extracellular ligand-binding domain, transmembrane a helices, and a C-terminal cytosolic domain. The cytosolic domains of cytokine receptors are generally devoid of any known catalytic activity. Cytokine receptors instead can function in association with non-receptor kinases (e.g., tyrosine kinases or threonine/serine kinases), which can be activated as a result of ligand binding to the receptor. The chimeric receptor can comprise at least an extracellular region (e.g., ligand binding domain) of a catalytic receptor that non-covalently associates with an intracellular kinase (e.g., a cytokine receptor), or any derivative, variant or fragment thereof. The chimeric receptor can comprise at least a membrane spanning region of a catalytic receptor that non-covalently associates with an intracellular kinase (e.g., a cytokine receptor), or any derivative, variant or fragment thereof. The chimeric receptor can comprise at least an intracellular region (e.g., cytosolic domain) of a catalytic receptor that non-covalently associates with an intracellular kinase (e.g., a cytokine receptor), or any derivative, variant or fragment thereof. The chimeric receptor comprising a catalytic receptor that non-covalently associates with an intracellular kinase, or any derivative, variant or fragment thereof, can recruit a binding partner. In some cases, ligand binding to the chimeric receptor comprising a catalytic receptor that non-covalently associates with an intracellular kinase, or any derivative, variant or fragment thereof, may result in a conformational change, chemical modification, or combination thereof, which recruits a binding partner to the receptor.

In some cases, the chimeric receptor can comprise a cytokine receptor, for example a type I cytokine receptor or a type II cytokine receptor, or any derivative, variant or fragment thereof. The chimeric receptor can comprise an interleukin receptor (e.g., IL-2R, IL-3R, IL-4R, IL-5R, IL-6R, IL-7R, IL-9R, IL-11R, IL-12R, IL-13R, IL-15R, IL-21R, IL-23R, IL-27R, and IL-31R), a colony stimulating factor receptor (e.g., erythropoietin receptor, CSF-1R, CSF-2R, GM-CSFR, and G-CSFR), a hormone receptor/neuropeptide receptor (e.g., growth hormone receptor, prolactin receptor, and leptin receptor), or any derivative, variant or fragment thereof. The chimeric receptor can comprise a type II cytokine receptor, or any derivative, variant or fragment thereof. The chimeric receptor can comprise an interferon receptor (e.g., IFNAR1, IFNAR2, and IFNGR), an interleukin receptor (e.g., IL-10R, IL-20R, IL-22R, and IL-28R), a tissue factor receptor (also called platelet tissue factor), or any derivative, variant or fragment thereof.

In some cases, the chimeric receptor comprising a cytokine receptor can bind an antigen comprising any suitable cytokine receptor ligand, or any derivative, variant or fragment thereof. Non-limiting examples of cytokine receptor ligands include interleukins (e.g., IL-2, IL-3, IL-4, IL-5, IL-6, IL-7, IL-9, IL-10, IL-11, IL-12, IL-13, IL-15, IL-20, IL-21, IL-22, IL-23, IL-27, IL-28, and IL-31), interferons (e.g., IFN-α, IFN-β, IFN-γ), colony stimulating factors (e.g., erythropoietin, macrophage colony-stimulating factor, granulocyte macrophage colony-stimulating factors or GM-CSFs, and granulocyte colony-stimulating factors or G-CSFs), and hormones (e.g., prolactin and leptin).

In some cases, the chimeric receptor can comprise a death receptor, a receptor containing a death domain, or any derivative, variant or fragment thereof. Death receptors are often involved in regulating apoptosis and inflammation. Death receptors include members of the TNF receptor family such as TNFR1, Fas receptor, DR4 (also known as TRAIL receptor 1 or TRAILR1) and DR5 (also known as TRAIL receptor 2 or TRAILR2). The chimeric receptor can comprise at least an extracellular region (e.g., ligand binding domain) of a death receptor, or any derivative, variant or fragment thereof. The chimeric receptor can comprise at least a membrane spanning region of a death receptor, or any derivative, variant or fragment thereof. The chimeric receptor can comprise at least an intracellular region (e.g., cytosolic) domain of a death receptor, or any derivative, variant or fragment thereof. The chimeric receptor polypeptide comprising a death receptor, or any derivative, variant or fragment thereof, can undergo receptor oligomerization in response to ligand binding, which in turn can result in the recruitment of specialized adaptor proteins and activation of signaling cascades, such as caspase cascades. The chimeric receptor can comprise a death receptor, or any derivative, variant or fragment thereof, results in a conformational change, chemical modification, or combination thereof, which recruits a binding partner to the chimeric receptor.

The chimeric receptor comprising a death receptor can bind an antigen comprising any suitable ligand of a death receptor, or any derivative, variant or fragment thereof. Non-limiting examples of ligands bound by death receptors include TNFα, Fas ligand, and TNF-related apoptosis-inducing ligand (TRAIL).

In some cases, the chimeric receptor can comprise an immune receptor, or any derivative, variant or fragment thereof. Immune receptors can include members of the immunoglobulin superfamily (IgSF) which share structural features with immunoglobulins, e.g., a domain known as an immunoglobulin domain or fold. IgSF members include, but are not limited to, cell surface antigen receptors, co-receptors and costimulatory molecules of the immune system, and molecules involved in antigen presentation to lymphocytes. The chimeric receptor can comprise at least an extracellular region (e.g., ligand binding domain) of an immune receptor, or any derivative, variant or fragment thereof. The chimeric receptor can comprise at least a region spanning a membrane of an immune receptor, or any derivative, variant or fragment thereof. The chimeric receptor can comprise at least an intracellular region (e.g., cytoplasmic domain) of an immune receptor, or any derivative, variant or fragment thereof. The chimeric receptor comprising an immune receptor, or any derivative, variant or fragment thereof, can recruit a binding partner. Ligand binding to a chimeric receptor comprising an immune receptor, or any derivative, variant or fragment thereof, can result in a conformational change, chemical modification, or combination thereof, which recruits a binding partner to the chimeric receptor.

In some cases, the chimeric receptor can comprise a cell surface antigen receptor such as a T cell receptor (TCR), a B cell receptor (BCR), or any derivative, variant or fragment thereof. T cell receptors generally comprise two chains, either the TCR-alpha and -beta chains or the TCR-delta and -gamma chains. A chimeric polypeptide comprising a TCR, or any derivative, variant or fragment thereof, can bind a major histocompatibility complex (MHC) protein. B cell receptors generally comprises a membrane bound immunoglobulin and a signal transduction moiety. A chimeric polypeptide comprising a BCR, or any derivative, variant or fragment thereof, can bind a cognate BCR antigen. A chimeric polypeptide comprising at least an immunoreceptor tyrosine-based activation motif (ITAM) found in the cytoplasmic domain of certain immune receptors. A chimeric polypeptide may comprise at least an immunoreceptor tyrosine-based inhibition motif (ITIM) found in the cytoplasmic domain of certain immune receptors. A chimeric polypeptide comprising ITAM and/or ITIM domains can be phosphorylated following ligand binding to an antigen interacting domain. The phosphorylated regions can serve as docking sites for other proteins involved in immune cell signaling.

The antigen interacting domain of a chimeric receptor can bind a membrane bound antigen, for example an antigen bound to the extracellular surface of a cell (e.g., a target cell). The antigen interacting domain may bind a non-membrane bound antigen, for example an extracellular antigen that is secreted by a cell (e.g., a target cell) or an antigen located in the cytoplasm of a cell. Antigens (e.g., membrane bound and non-membrane bound) can be associated with a disease such as a viral, bacterial, and/or parasitic infection; inflammatory and/or autoimmune disease; or neoplasm such as a cancer and/or tumor. Cancer antigens, for example, may be proteins produced by tumor cells that can elicit an immune response, particularly a T-cell mediated immune response. The selection of the antigen binding portions of a chimeric receptor can depend on the particular type of cancer antigen to be targeted. In some cases, the tumor antigen may comprise one or more antigenic cancer epitopes associated with a malignant tumor. Malignant tumors can express a number of proteins that can serve as target antigens for an immune attack. The antigen interaction domains can bind to cell surface signals, extracellular matrix (ECM), paracrine signals, juxtacrine signals, endocrine signals, autocrine signals, signals that can trigger or control genetic programs in cells, or any combination thereof. In some cases, interactions between the cell signals that bind to the chimeric receptor involve a cell-cell interaction, cell-soluble chemical interaction, and cell-matrix or microenvironment interaction.

### GMP and Actuator Moiety

The GMP may comprise an actuator moiety that regulates expression of a target polynucleotide in the cell. The target polynucleotide in the cell may encode a target polypeptide. In some cases, the target polypeptide may induce or inhibit proliferation, differentiation, and/or survival of the cell. The actuator moiety can bind to a target polynucleotide to regulate expression and/or activity of a target gene encoded by the target polynucleotide. In some embodiments, the target polynucleotide comprises genomic DNA. In some embodiments, the target polynucleotide comprises a region of a plasmid, for example a plasmid carrying an exogenous gene. In some embodiments, the target polynucleotide comprises RNA, for example mRNA. In some embodiments, the target polynucleotide comprises an endogenous gene or gene product. The actuator moiety can comprise a nuclease (e.g., DNA nuclease and/or RNA nuclease), modified nuclease (e.g., DNA nuclease and/or RNA nuclease) that is nuclease-deficient or has reduced nuclease activity compared to a wild-type nuclease or a variant thereof. The actuator moiety can regulate expression or activity of a gene and/or edit the sequence of a nucleic acid (e.g., a gene and/or gene product). In some embodiments, the actuator moiety comprises a DNA nuclease such as an engineered (e.g., programmable or targetable) DNA nuclease to induce genome editing of a target DNA sequence. In some embodiments, the actuator moiety comprises a RNA nuclease such as an engineered (e.g., programmable or targetable) RNA nuclease to induce editing of a target RNA sequence. In some embodiments, the actuator moiety has reduced or minimal nuclease activity (e.g., dCas). An actuator moiety having reduced or minimal nuclease activity can regulate expression and/or activity of a gene by physical obstruction of a target polynucleotide or recruitment of additional factors effective to suppress or enhance expression of the target polynucleotide. The actuator moiety can physically obstruct the target polynucleotide or recruit additional factors effective to suppress or enhance expression of the target polynucleotide.

In some cases, the actuator moiety may comprise a heterologous functional domain (e.g., a transcription activator, a transcription repressor, a chromosome modification enzyme, etc.). In some cases, the actuator moiety comprises an activator effective to increase expression of the target polynucleotide. In some embodiments, the actuator moiety comprises a transcriptional activator effective to increase expression of the target polynucleotide. In other cases, the actuator moiety comprises a repressor effective to decrease expression of the target polynucleotide. Non-limiting examples of transcription activators include GAL4, VP16, VP64, p65 subdomain (NFkappaB), and VP64-p65-Rta (VPR). In some embodiments, the actuator moiety comprises a transcriptional repressor effective to decrease expression of the target polynucleotide. Non-limiting examples of transcription repressors include Kruippel associated box (KRAB or SKD), the Mad mSIN3 interaction domain (SID), and the ERF repressor domain (ERD). In some embodiments, the actuator moiety comprises a nuclease-null DNA binding protein derived from a DNA nuclease that can induce transcriptional activation or repression of a target DNA sequence. In some embodiments, the actuator moiety comprises a nuclease-null RNA binding protein derived from a RNA nuclease that can induce transcriptional activation or repression of a target RNA sequence. In some embodiments, the actuator moiety is a nucleic acid-guided actuator moiety. In some embodiments, the actuator moiety is a DNA-guided actuator moiety. In some embodiments, the actuator moiety is an RNA-guided actuator moiety or a variant thereof, which RNA-guided actuator moiety forms a complex with the target polynucleotide. An actuator moiety can regulate expression or activity of a gene and/or edit a nucleic acid sequence, whether exogenous or endogenous.

Any suitable nuclease can be used. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), and eukaryotic Argonaute (eAgo)); and any variant thereof. In some cases, the actuator moiety is a CRISPR-associated (Cas) protein or a fragment thereof that substantially lacks DNA cleavage activity (dCas). In some cases, the actuator moiety can be Cas9 and/or Cpf1.

Any target gene can be regulated by the comprising the actuator moiety. It is contemplated that genetic homologues of a gene described herein are covered. For example, a gene can exhibit a certain identity and/or homology to genes disclosed herein. Therefore, it is contemplated that the expression of a gene that exhibits or exhibits at least about 50%, 55%, 60%, 65%,70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology (at the nucleic acid or protein level) can be regulated. It is also contemplated that the expression of a gene that exhibits or exhibits at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity (at the nucleic acid or protein level) can be regulated.

The target polypeptide may encode a peptide or a protein that is immune related (e.g., related to survival, proliferation, differentiation, activity, identification, etc. or an immune cell, such as a T cell). The target polypeptide may encode a peptide or protein involved in immune cell regulation. In some cases, the target polypeptide may be PD-1, PD-L1, and/or CTLA-4.

The target polypeptide may encode a peptide or a protein that is immune related (e.g., related to survival, proliferation, differentiation, activity, identification, etc. or an immune cell, such as a T cell). The target polypeptide may encode a peptide or protein involved in immune cell regulation. In some cases, the target polypeptide may be PD-1, PD-L1, and/or CTLA-4.

### Administration of GMP

In some cases, administration of the GMP to the cell can comprise treating the cell with a delivery vehicle, which delivery vehicle comprises at least a portion of the GMP and/or a polynucleotide that encodes at least a portion of the GMP. The delivery vehicle may be viral or non-viral. The at least the portion of the GMP and/or the polynucleotide that encodes the at least the portion of the GMP may be attached covalently and/or non-covalently (e.g., ionically, via hydrogen bonds, etc.) to the delivery vehicle. Alternatively or in addition to, the at least the portion of the GMP and/or the polynucleotide that encodes the at least the portion of the GMP may be encapsulated by the delivery vehicle without any physical attachment to the delivery vehicle.

In some cases, the delivery vehicle may comprise a targeting moiety with an affinity to one or more ligands (e.g., a portion of a cell surface receptor, a polysaccharide chain, one or more extracellular proteins) present on or adjacent to the surface of the cell. The targeting moiety may enhance targeting and binding of the delivery vehicle to the cell. The targeting moiety may enhance intracellular entrance, uptake, and/or penetration of the delivery vehicle into the cell. The targeting moiety may be linked (e.g., via covalent and/or a non-covalent bond) to an external surface of the delivery vehicle. The targeting moiety may be a non-natural molecule, at least a portion of a natural molecule, a functional derivative thereof, or a combination thereof. The targeting moiety may be a small molecule, a polynucleotide (e.g., an aptamer), a polypeptide (e.g., an oligopeptide or a protein), an antibody or a functional fragment thereof, a functional derivative thereof, or a combination thereof.

In some cases, the delivery vehicle may not comprise such targeting moiety against the cell.

Examples of the viral delivery vehicle may comprise an adenovirus, a retrovirus, a lentivirus (e.g., a human immunodeficiency virus (HIV)), an adeno-associated virus (AAV), and/or a Herpes simplex virus (HSV). In an example, the viral delivery vehicle may be a retrovirus. The retrovirus may be a gamma-retrovirus selected from the group consisting of: Feline Leukemia Virus (FLV), Feline Sarcoma Virus (Strain Hardy-Zuckerman 4), Finkel-Biskis-Jinkins Murine Sarcoma Virus (FBJMSV), Murine leukemia virus (MLV) (e.g. Friend Murine Leukemia Virus (FMIV), Moloney Murine Leukemia Virus (MMLV), Murine Type C Retrovirus (MTCR)), Gibbon Ape Leukemia Virus (GALV), Koala Retrovirus (KR), Moloney Murine Sarcoma Virus (MMSV), Porcine Endogenous Retrovirus E (PERE), Reticuloendotheliosis Virus (RV), Woolly Monkey Sarcoma Virus (WMSV), Baboon Endogenous Virus Strain M7 (BEVSM7), Murine Osteosarcoma Virus (MOV), Mus Musculus Mobilized Endogenous Polytropic Provirus (MMMEPP), PreXMRV-1, RD114 Retrovirus, Spleen Focus-Forming Virus (SFFV), Abelson murine leukemia virus (AMLV), Murine Stem Cell Virus (MSCV), and variants thereof.

The delivery vehicle may comprise of a nucleotide (e.g., a polynucleotide), an amino acid (e.g., a peptide or polypeptide), a polymer, a metal, a ceramic, a derivative thereof, or a combination thereof. In an example, the delivery vehicle may comprise of a diamond nanoparticle (“nanodiamonds”), a gold nanoparticle, a silver nanoparticle, a calcium phosphate nanoparticle, etc. The delivery vehicle may or may not comprise a fluid (e.g., a liquid or gas). The delivery vehicle may have various shapes and sizes. For example, the delivery vehicle may be in the shape of a sphere, cuboid, or disc, or any partial shape or combination of shapes thereof. The delivery vehicle may have a cross-section that is circular, triangular, square, rectangular, pentagonal, hexagonal, or any partial shape or combination of shapes thereof.

Examples of the non-viral delivery vehicle may comprise nanoparticles, nanospheres, nanocapsules, microparticies, microspheres, microcapsules, liposomes, nanoemulsions, solid lipid nanoparticles, modifications thereof, or combinations thereof. The non-viral delivery vehicle of the present invention may be prepared by methods, such as, but not limited to, nanoprecipitation, emulsion solvent evaporation method, emulsion-crosslinking method, emulsion solvent diffusion method, microemulsion method, gas antisolvent precipitation method, ionic gelation methods milling or size reduction method, PEGylation method, salting-out method, dialysis method, single or double emulsification method, nanospray drying method, layer by layer method, desolvation method, supercritical fluid technology, supramolecular assembly, or combinations thereof.

In some cases, the method can further comprise integrating into the genome of the cell a nucleic acid sequence (e.g., a polynucleotide) encoding at least a portion of the first chimeric polypeptide and/or the second chimeric polypeptide, as provided herein in the present disclosure. In some cases, the nucleic acid sequence may encode at least a portion of the GMP. In some cases, the nucleic acid sequence (e.g., a polynucleotide) encoding the at least the portion of the first and/or second chimeric polypeptides may be integrated into the genome of the cell. Upon administration of the nucleic acid encoding the at least the portion of the first and/or second chimeric polypeptides (e.g., with or without the delivery vehicle), at least a portion of the nucleic acid may be integrated into the genome of the cell. The at least the portion of the integrated nucleic acid may be placed under the control of an autologous promoter of the cell. Alternatively or in addition to, the at least a portion of the integrated nucleic acid may further comprise a promoter that is autologous or heterologous (e.g., a heterologous promoter) to the cell. The heterologous promoter may be configured to bind one or more molecules (e.g., an RNA polymerase, a transcription factor, etc.) that are homologous or heterologous to the cell.

The cell may be in vivo and/or ex vivo (e.g., in vitro) during the treatment with the delivery vehicle comprising a payload (e.g., the at least the portion of the first and/or second chimeric polypeptides, the nucleic acid that encodes the at least the portion of the first and/or second chimeric polypeptides, etc.).

In some cases, the delivery vehicle comprising the payload may be injected into a bodily part of a subject (e.g., a vein, a marrow, etc. of a patient), and the delivery vehicle may interact with (e.g., enter into) the cell in vivo. Other examples of the injection method may include intradermal, subcutaneous, intramuscular, intravenous, intraosseous, intraperitoneal, intrathecal, epidural, intracardiac, intraarticular, intracavernous, and/or intravitreal.

In some cases, the subject may be injected with a dose of the delivery vehicle comprising the payload for at least about 1, 2, 3, 4, 5, 6, 7, 8, 9, 10, or more times. In some cases, the subject may be injected with a dose of the delivery vehicle comprising the payload for at most about 10, 9, 8, 7, 6, 5, 4, 3, 2, or 1 time. In some cases, the subject may be injected with a dose of the delivery vehicle comprising the payload at a frequency of at least once every 1, 2, 3, 4, 5, 6, 7, 8, 9, 10, 20, 30, 60, 90, 180, 360, or more days. In some cases, the subject may be injected with a dose of the delivery vehicle comprising the payload at a frequency of at most once every 360, 180, 90, 60, 30, 20, 10, 9, 8, 7, 6, 5, 4, 3, 2, or 1 day.

In some cases, the cell may be isolated from the subject, and the isolated cell may be treated (e.g., cultured in a culture media) with the delivery vehicle comprising the payload. The isolated cell may be allowed or stimulated to proliferate prior to, during, and/or subsequent to the treatment with the delivery vehicle comprising the payload. In some cases, the cell of interest may be an immune cell. In such a case, the immune cell (e.g., a T cell) may be isolated from the subject. Alternatively or in addition to, a cell that is not the immune cell (e.g., a stem cell, a skin cell, etc.) may be isolated from the subject, and the isolated cell may be induced to differentiate into the immune cell, trans-differentiate into the immune cell, and/or express one or more markers (e.g., one or more TCR complexes) indicative of the immune cell prior to the treatment with the delivery vehicle comprising a payload. In some cases, the cell that is not the immune cell may first be de-differentiated into an induced pluripotent stem cell (iPSC) prior to differentiation into the immune cell (e.g., the T cell) and/or inducing expression of the one or more TCR complexes. Following, the isolated and treated cell may be injected (transplanted) into the subject.

Any of the cells provided herein that are treated (ex vivo and/or in vivo) with at least the payload to administer the GMR comprising the actuator moiety may be referred to as an engineered cell (e.g., an engineered immune cell, such as an engineered T cell).

In some cases, such engineered cell may be injected into a bodily part of a subject (e.g., a vein, a marrow, etc. of a patient), and the delivery vehicle may interact with (e.g., enter into) the cell in vivo. Other examples of the injection method may include intradermal, subcutaneous, intramuscular, intravenous, intraosseous, intraperitoneal.

In some cases, the subject may be injected with a dose of the engineered cells (e.g., cells administered with the GMP) for at least about 1, 2, 3, 4, 5, 6, 7, 8, 9, 10, or more times. In some cases, the subject may be injected with a dose of the engineered cells for at most about 10, 9, 8, 7, 6, 5, 4, 3, 2, or 1 time. In some cases, the subject may be injected with a dose of the engineered cells at a frequency of at least once every 1, 2, 3, 4, 5, 6, 7, 8, 9, 10, 20, 30, 60, 90, 180, 360, or more days. In some cases, the subject may be injected with a dose of the engineered cells at a frequency of at most once every 360, 180, 90, 60, 30, 20, 10, 9, 8, 7, 6, 5, 4, 3, 2, or 1 day.

In some cases, the subject may be injected with at least about 0.5, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19, 20, 30, 40, 50, 60, 70, 80, 90, 100 (x 10⁹) of the engineered cells, or more. In other cases, the subject may be injected with at most about 100, 90, 80, 70, 60, 50, 40, 30, 20, 19, 18, 17, 16, 15, 14, 13, 12, 11, 10, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5 (x 10⁹) of the engineered cells, or less.

In some cases, the GMP may be a portion of a chimeric polypeptide. The chimeric polypeptide may or may not be a transmembrane protein. In an example, the chimeric polypeptide may be a CAR, and the GMP may be at least a portion of an intracellular domain of the CAR. In another example, the chimeric polypeptide may be a chimeric transmembrane protein, and the GMP may be at least a portion of an intracellular domain of the chimeric transmembrane protein. In a different example, the chimeric polypeptide comprising the GMP may be an intracellular protein.

In some cases, the administration of the GMP to the cell can comprise treating the cell with at least a portion of the chimeric polypeptide comprising the GMP and/or a polynucleotide that encodes the at least a portion of the chimeric polypeptide comprising the GMP. Such treatment may occur in the presence or absence of one or more delivery vehicles provided herein in the present disclosure. In some cases, the method can further comprise administering to the cell a chimeric polypeptide comprising the GMP, wherein the chimeric polypeptide is operable to release the GMP from the chimeric polypeptide in response to a stimulant (e.g., the ligand of the receptor provided herein in the present disclosure), and wherein the released GMP is operable to regulate expression of the target polynucleotide in the cell. In some cases, the method can further comprise administering to the cell a chimeric polypeptide comprising the GMP and a nuclear localization domain, wherein the nuclear localization domain is operable to translocate the chimeric polypeptide to a nucleus of the cell in response to a stimulant, and wherein the translocated GMP is operable to regulate expression of the target polynucleotide in the cell.

In some cases, the nuclear localization domain can be derived from a transcription factor, as abovementioned. The transcription factor can be a regulatable transcription factor that is only active and able to translocate into a nucleus in response to a signal or signaling pathway. The transcription factor can be a regulatable transcription factor that is primarily active and able to translocate into a nucleus in response to a signal or signaling pathway. The transcription factor can be a regulatable transcription factor that is generally active and able to translocate into a nucleus in response to a signal or signaling pathway.

In some examples, the nuclear localization domain can be derived from the NFAT family members (e.g., NFATp, NFAT1, NFATc1, NFATc2, NFATc3, NFAT4, NFATx, NFATc4, NFAT3, and NFAT5), nuclear factor kappa B (NF-κB), NFKB1 p50, activator protein 1 (AP-1), signal transducer and activator of transcription family members (e.g., STAT1, STAT2, STAT3, STAT4, STAT5A, STAT5B, and STAT6), sterol response element-binding proteins (e.g., SREBP-1 and SREBF1), a light or circadian or electromagnetic sensing protein such as cryptochromes (e.g., CRY1, CRY2), Timeless (TIM), PAS domain of PER proteins (e.g., PER1, PER2, and PER3), or other transcription factors or signal transducers.

In some cases, the GMP can regulate expression of the target polynucleotide in the cell by at least a 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, 1000-fold, or more in comparison to the cell in the absence of the GMP. In some cases, the GMP can regulate expression of the target polynucleotide in the cell by at most 1000-fold, 100-fold, 10-fold, 5-fold, 4.0-fold, 3.5-fold, 3.0-fold, 2.5-fold, 2.0-fold, 1.9-fold, 1.8-fold, 1.7-fold, 1.6-fold, 1.5-fold, 1.4-fold, 1.3-fold, 1.2-fold, 1.1-fold, or less in comparison to the cell in the absence of the GMP.

In some cases, the GMP can regulate expression of the target polynucleotide in the cell for at least 1 minute, 5 minutes, 10 minutes, 30 minutes, 1 hour, 2 hours, 3 hours, 4 hours, 5 hours, 6 hours, 7 hours, 8 hours, 12 hours, 16 hours, 20 hours, 24 hours, 2 days, 3 days, 4 days, 5 days, 6 days, 7 days, 2 weeks, 3 weeks, 4 weeks, 2 months, 4 months, 6 months, 1 year, or more in comparison to the cell in the absence of the GMP. In some cases, the GMP can regulate expression of the target polynucleotide in the cell for at most 1 year, 6 months, 4 months, 2 months, 4 weeks, 3 weeks, 2 weeks, 7 days, 6 days, 5 days, 4 days, 3 days, 2 days, 24 hours, 20 hours, 16 hours, 12 hours, 8 hours, 7 hours, 6 hours, 5 hours, 4 hours, 3 hours, 2 hours, 1 hour, 30 minutes, 10 minutes, 5 minutes, 1 minute, or less in comparison to the cell in the absence of the GMP.

The regulating the expression of the target polynucleotide in the cell can comprise decreasing, increasing, inhibiting, and/or prolonging the expression of the target polynucleotide in the cell. The regulating the expression of the target polynucleotide in the cell can be decreasing the expression of the target polynucleotide in the cell. The regulating the expression of the target polynucleotide in the cell can be increasing the expression of the target polynucleotide in the cell.

The regulating the expression of the target polynucleotide in the cell may directly and/or indirectly allow the regulating the activity of the cell. In some cases, the regulating the activity of the cell can comprise decreasing and/or inhibiting self-inflicted injury of the cell, death of the cell by another cell, and/or death of another cell by the cell, thereby improving (directly and/or indirectly) viability, proliferation, and/or function of the cell.

In some cases, the regulating the activity of the cell can comprise inducing and/or prolonging activation of the cell (e.g., activation of the immune cell, such as the T cell). The activation of the cell can comprise activation of one or more biological activities (e.g., migration, proliferation, synthesis of one or more polypeptides, etc.) of the cell.

In some cases, the GMP may be configured to reduce and/or prevent activation of the cell.

In some cases, the GMP comprising the actuator moiety may be configured to increase or decrease expression of one or more angiogenic factors in the cell. In some cases, the GMP comprising the actuator moiety may be configured to decrease expression of one or more angiogenic factors in the cell. In some cases, the GMP comprising the actuator moiety may be configured to decrease expression of one or more angiogenic factors in the cell. The GMP comprising the actuator moiety may be expressed along with a guide RNA (e.g., sgRNA) against one or more polynucleotide sequences encoding for the one or more angiogenic factors in the T cell. The actuator moiety of the GMP, in conjunction with the guide RNA, may be configured to increase or decrease expression of one or more angiogenic factors in the cell.

The one or more angiogenic factors can include pro-angiogenic factors and/or anti-angiogenic factors. Examples of the pro-angiogenic factors can include, but are not limited to, FGF, VEGF, VEGFR, NRP-1, Ang1, Ang2, PDGF (BB-homodimer), PDGFR, TGF-β, endoglin, TGF-βreceptors, MCP-1, Integrins αvβ3, αvβ₃, α₅β₁, VE-Cadherin, CD31, ephrin, plasminogen activators, plasminogen activator inhibitor-1, eNOS, COX-2, AC133, Id1/Id3, Angiogenin, HGF, Vegf, IL-17, IL-1 alpha, IL-8, IL-6, Cxcl5, Fgfα, Fgfβ, Tgfα, Tgfβ, MMPs (including mmp9), Plasminogen activator inhibitor-1, Thrombospondin, Angiopoietin 1, Angiopoietin 2, Amphiregulin, Leptin, Endothelin-1, AAMP, AGGF1, AMOT, ANGLPTL3, ANGPTL4, BTG1, IL-1β, NOS3, TNFSF12, and/or VASH2.

In some cases, a nucleic acid sequence encoding the GMP may be integrated into a genome of the cell.

In some cases, the cleavage recognition site may comprise a polypeptide sequence, and the cleavage moiety may comprise protease activity. In some cases, the cleavage recognition site may comprise a disulfide bond, and the cleavage moiety may comprise oxidoreductase activity. In some cases, the cleavage recognition site may comprise a first portion of an intein sequence that reacts with a second portion of the intein sequence to release the actuator moiety.

In some cases, the cleavage moiety can cleave the recognition site when in proximity to the cleavage recognition site. The cleavage recognition site can comprise a polypeptide sequence that is a recognition sequence of a protease. The cleavage moiety can comprise protease activity which recognizes the polypeptide sequence. A cleavage moiety comprising protease activity can be a protease, or any derivative, variant or fragment thereof. A protease can refer to any enzyme that performs proteolysis, in which polypeptides are cleaved into smaller polypeptides or amino acids. Various proteases can be suitable for use as a cleavage moiety. Some proteases can be highly promiscuous such that a wide range of protein substrates are hydrolysed. Some proteases can be highly specific and only cleave substrates with a certain sequence, e.g., a cleavage recognition sequence or peptide cleavage domain. In some cases, the cleavage recognitions site can comprise multiple cleavage recognition sequences, and each cleavage recognition sequence can be recognized by the same or different cleavage moiety comprising protease activity (e.g., protease). Sequence-specific proteases that can be used as cleavage moieties include, but are not limited to, superfamily CA proteases, e.g., families C1, C2, C6, C10, C12, C16, C19, C28, C31, C32, C33, C39, C47, C51, C54, C58, C64, C65, C66, C67, C70, C71, C76, C78, C83, C85, C86, C87, C93, C96, C98, and C101, including papain (Carica papaya), bromelain (Ananas comosus), cathepsin K (liverwort) and calpain (Homo sapiens); superfamily CD proteases, e.g., family C11, C13, C14, C25, C50, C80, and C84: such as caspase-1 (Rattus norvegicus) and separase (Saccharomyces cerevisiae); superfamily CE protease, e.g., family C5, C48, C55, C57, C63, and C79 including adenain (human adenovirus type 2); superfamily CF proteases, e.g., family C15 including pyroglutamyl-peptidase I (Bacillus amyloliquefaciens); superfamily CL proteases, e.g., family C60 and C82 including sortase A (Staphylococcus aureus); superfamily CM proteases, e.g. family C18 including hepatitis C virus peptidase 2 (hepatitis C virus); superfamily CN proteases, e.g., family C9 including sindbis virus-type nsP2 peptidase (sindbis virus); superfamily CO proteases, e.g., family C40 including dipeptidyl-peptidase VI (Lysinibacillus sphaericus); superfamily CP proteases, e.g., family C97 including DeSI-1 peptidase (Mus musculus); superfamily PA proteases, e.g., family C3, C4, C24, C30, C37, C62, C74, and C99 including TEV protease (Tobacco etch virus); superfamily PB proteases, e.g., family C44, C45, C59, C69, C89, and C95 including amidophosphoribosyltransferase precursor (homo sapiens); superfamily PC proteases, families C26, and C56 including [symbol] -glutamyl hydrolase (Rattus norvegicus); superfamily PD proteases, e.g., family C46 including Hedgehog protein (Drosophila melanogaster); superfamily PE proteases, e.g., family P1 including DmpA aminopeptidase (Ochrobactrum anthropi); others proteases, e.g., family C7, C8, C21, C23, C27, C36, C42, C53 and C75. Additional proteases include serine proteases, e.g., those of superfamily SB, e.g., families S8 and S53 including subtilisin (Bacillus licheniformis); those of superfamily SC, e.g., families S9, S10, S15, S28, S33, and S37 including prolyl oligopeptidase (Sus scrofa); those of superfamily SE, e.g., families S11, S12, and S13 including D-Ala-D-Ala peptidase C (Escherichia coli); those of superfamily SF, e.g., families S24 and S26 including signal peptidase I (Escherichia coli); those of Superfamily SJ, e.g., families S16, S50, and S69 including lon-A peptidase (Escherichia coli); those of Superfamily SK, e.g., families S14, S41, and S49 including Clp protease (Escherichia coli); those of Superfamily SO, e.g., families S74 including Phage K1F endosialidase CIMCD self-cleaving protein (Enterobacteria phage K1F); those of superfamily SP, e.g., family S59 including nucleoporin 145 (Homo sapiens); those of superfamily SR, e.g., family S60 including Lactoferrin (Homo sapiens); those of superfamily SS, families S66 including murein tetrapeptidase LD-carboxypeptidase (Pseudomonas aeruginosa); those of superfamily ST, e.g., families S54 including rhomboid-1 (Drosophila melanogaster); those of superfamily PA, e.g., families S1, S3, S6, S7, S29, S30, S31, S32, S39, S46, S55, S64, S65, and S75 including Chymotrypsin A (Bos taurus); those of superfamily PB, e.g., families S45 and S63 including penicillin G acylase precursor (Escherichia coli); those of superfamily PC, e.g., families S51 including dipeptidase E (Escherichia coli); those of superfamily PE, e.g., families P1 including DmpA aminopeptidase (Ochrobactrum anthropi); those unassigned, e.g., families S48, S62, S68, S71, S72, S79, and S81 threonine proteases, e.g., those of superfamily PB clan, e.g., families T1, T2, T3, and T6 including archaean proteasome, ȕ component (Thermoplasma acidophilum); and those of superfamily PE clan, e.g., family T5 including ornithine acetyltransferase (Saccharomyces cerevisiae); aspartic proteases, e.g., BACE1, BACE2; cathepsin D; cathepsin E; chymosin; napsin-A; nepenthesin; pepsin; plasmepsin; presenilin; renin; and HIV-1 protease, and metalloproteinases, e.g., exopeptidases, metalloexopeptidases; endopeptidases, and metalloendopeptidases. A cleavage recognition sequence (e.g., polypeptide sequence) can be recognized by any of the proteases disclosed herein.

In some cases, the cleavage recognition site can comprise a cleavage recognition sequence (e.g., polypeptide sequence or peptide cleavage domain) that is recognized by a protease selected from the group consisting of: achromopeptidase, aminopeptidase, ancrod, angiotensin converting enzyme, bromelain, calpain, calpain I, calpain II, carboxypeptidase A, carboxypeptidase B, carboxypeptidase G, carboxypeptidase P, carboxypeptidase W, carboxypeptidase Y, caspase 1, caspase 2, caspase 3, caspase 4, caspase 5, caspase 6, caspase 7, caspase 8, caspase 9, caspase 10, caspase 11, caspase 12, caspase 13, cathepsin B, cathepsin C, cathepsin D, cathepsin E, cathepsin G, cathepsin H, cathepsin L, chymopapain, chymase, chymotrypsin, clostripain, collagenase, complement C1r, complement C1s, complement Factor D, complement factor I, cucumisin, dipeptidyl peptidase IV, elastase (leukocyte), elastase (pancreatic), endoproteinase Arg-C, endoproteinase Asp-N, endoproteinase Glu-C, endoproteinase Lys-C, enterokinase, factor Xa, ficin, furin, granzyme A, granzyme B, HIV Protease, IGase, kallikrein tissue, leucine aminopeptidase (general), leucine aminopeptidase (cytosol), leucine aminopeptidase (microsomal), matrix metalloprotease, methionine, aminopeptidase, neutrase, papain, pepsin, plasmin, prolidase, pronase E, prostate specific antigen, protease alkalophilic from Streptomyces griseus, protease from Aspergillus, protease from Aspergillus saitoi, protease from Aspergillus sojae, protease (B. licheniformis) (alkaline or alcalase), protease from Bacillus polymyxa, protease from Bacillus sp, protease from Rhizopus sp., protease S, proteasomes, proteinase from Aspergillus oryzae, proteinase 3, proteinase A, proteinase K, protein C, pyroglutamate aminopeptidase, rennin, rennin, streptokinase, subtilisin, thermolysin, thrombin, tissue plasminogen activator, trypsin, tryptase and urokinase.

Further details of proteases and associated recognition sequences that can be used in systems and methods of the present disclosure are disclosed in Patent Cooperation Treaty (PCT) Patent Application No. PCT/US17/012885 and PCT Patent Application No. PCT/US17/012881, each of which is incorporated in its entirety herein by reference.

In some cases, the actuator moiety of the GMP can be an RNA-guided actuator moiety or a variant thereof, which RNA-guided actuator moiety forms a complex with the target polynucleotide. In some cases, the actuator moiety can be a CRISPR-associated (Cas) protein or a fragment thereof that substantially lacks DNA cleavage activity. In some cases, the actuator moiety can be Cas9 and/or Cpf1. In some cases, the actuator moiety can comprise an activator effective to increase expression of the target polynucleotide. In some cases, the actuator moiety can comprise a repressor effective to decrease expression of the target polynucleotide.

Further details of design and application of systems comprising the chimeric polypeptide (e.g., chimeric receptor polypeptide, the chimeric adaptor polypeptide, etc.), CAR, GMP, ligands (e.g., antigens), modifications thereof, and expression cassettes comprising thereof are disclosed in Patent Cooperation Treaty Patent Application No. PCT/US17/012885, Patent Cooperation Treaty Patent Application No. PCT/US17/012881, Patent Cooperation Treaty Patent Application No. PCT/US18/041704, U.S. Pat. No. 9,856,497, U.S. Non-Provisional Application No. 15/806,756, U.S. Non-Provisional Application No. 16/029,299, U.S. Non-Provisional Application No. 16/029,299, U.S. Provisional Application No. 62/639,427, U.S. Provisional Application No. 62/639,386, Patent Cooperation Treaty Patent Application No. PCT/US19/023721, and U.S. Provisional Application No. 62/799,456, each of which is incorporated in its entirety herein by reference.

Contacting the cell with a ligand (e.g., an exogenous ligand) can occur directly and/or indirectly. Direct stimulation may occur when the ligand binds a portion of the cell. In some cases, the ligand may bind to the receptor of the cell. In an example, the ligand may bind to a ligand binding domain of the receptor. Indirect stimulation can occur when the ligand activates or deactivates a different cell, which different cell is operable to activate the cell by using its cell surface marker (e.g., a cell surface ligand) to bind the receptor of the cell. Consequentially, the cell may be activated to regulate expression of the target polynucleotide in the cell. The different cell may be of the same (e.g., another cell of the same type) or different cell type than the cell.

Contacting the cell with the ligand may occur prior to, during, and/or subsequent to administration of the GMP comprising the actuator moiety to the cell. The cell may be ex vivo and/or in vivo during the contacting of the cell (e.g., the receptor of the cell) with the ligand.

Contacting the cell with the ligand may occur prior to, during, and/or subsequent to administration of the cell (e.g., the engineered cell) to a subject. The cell may be contacted with the ligand prior to, during, and/or subsequent to administration of the cell to the subject for a duration of time of at least about 0.5, 1, 1.5, 2, 2.5, 3, 3.5, 4, 4.5, 5, 5.5, 6, 6.5 days, or more. The cell may be contacted with the ligand prior to, during, and/or subsequent to administration of the cell to the subject for a duration of time of at most about 6.5, 6, 5.5, 5, 4.5, 4, 3.5, 3, 2.5, 2, 1.5, 1, 0.5 days, or less. The cell may be contacted with the ligand for a duration of time of at least about 0.5, 1, 1.5, 2, 2.5, 3, 3.5, 4, 4.5, 5, 5.5, 6, 6.5 days, or more prior to administration of the cell to the subject. The cell may be contacted with the ligand for a duration of time of at most about 6.5, 6, 5.5, 5, 4.5, 4, 3.5, 3, 2.5, 2, 1.5, 1, 0.5 days, or less prior to administration of the cell to the subject. The cell may be contacted with the ligand for a duration of time of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 days, or more subsequent to administration of the cell to the subject. The cell may be contacted with the ligand for a duration of time of at most about 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10 days, or less subsequent to administration of the cell to the subject. In some cases, the cell may be contacted with the ligand at least about 1, 2, 3, 4, 5, 6, 7, 8, 9, 10 times, or more. In some cases, the cell may be contacted with the ligand at most about 10, 9, 8, 7, 6, 5, 4, 3, 2, or 1 time. In some cases, the cell may be contacted with the ligand at a dose concentration of at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 900, 1000 international units per milliliter (IU/mL), or more. In other cases, the cell may be contacted with the ligand at a dose concentration of at most about 1000, 900, 800, 790, 780, 770, 760, 750, 740, 730, 720, 710, 700, 690, 680, 670, 660, 650, 640, 630, 620, 610, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, 10 IU/mL, or less

In some cases, the ligand (i.e., the stimulant) of the receptor of the cell may be selected from the group consisting of interleukins (e.g., IL-2), interferons, transforming growth factors (TGFs), ligands for cluster of differentiation (CD) receptors, and variants thereof. The stimulant may be an antigen described in the subject disclosure. In some examples, the antigen may induce migration, survival, proliferation, and/or differentiation of an immune cell (e.g., a T cell). In some cases, the stimulant may comprise a vaccine (e.g., an immune cell vaccine). A vaccine may be a pharmaceutical composition comprising at least one immunologically protective molecule that induces an immunological and/or protective response in a cell (e.g., an immune cell) or an animal. A vaccine may further comprise one or more additional components (e.g., adjuvants) that enhance the immunological activity. In an example, the immune cell vaccine may be a peptide vaccine (e.g., p-27L) or a viral vaccine (e.g., p-210M, rFP-210M).

In some cases, the ligand binding domain (e.g., the stimulant binding domain) of the cell binds an antigen that is not membrane bound (e.g., non-membrane-bound), for example an extracellular antigen that is secreted by a cell (e.g., a target cell) or an antigen located in the cytoplasm of a cell (e.g., a target cell). Antigens (e.g., membrane bound and non-membrane bound) can be associated with a disease such as a viral, bacterial, and/or parasitic infection; inflammatory and/or autoimmune disease; or neoplasm such as a cancer and/or tumor. Non-limiting examples of antigens which can be bound by a ligand binding domain of a chimeric transmembrane receptor polypeptide of a subject system include, but are not limited to, 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β 3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-1a), carbonic anhydrase 9 (CA-IX), Carcinoembryonic antigen (CEA), cardiac myosin, CCCTC-Binding Factor (CTCF), CCL11 (eotaxin-1), CCR4, CCR5, CD11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2 receptor), CD27, CD274, CD28, CD3, CD3 ε, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B 1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFR_{V}III), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein α (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-1β, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-I receptor), insulin-like growth factor 2 (ILGF2), integrin α4β7, integrin β2, integrin α2, integrin α4, integrin α5β1, integrin α7β7, integrin αIIbβ3, integrin αvβ3, interferon α/β receptor, interferon γ-induced protein, Interleukin 11 receptor α (IL-11Rα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIR1), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R α), platelet-derived growth factor receptor β (PDGFR-β), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, β Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member 1A (XAGE1), β-amyloid, and κ-light chain, and variants thereof.

In some embodiments, the ligand binding domain binds an antigen selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R170I, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-⅟Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain. In some embodiments, the ligand binding domain binds to a tumor associated antigen.

In some embodiments, the target polynucleotide encodes for a cytokine. Non-limiting examples of cytokines include 4-1BBL, activin βA, activin βB, activin βC, activin βE, artemin (ARTN), BAFF/BLyS/TNFSF138, BMP10, BMP15, BMP2, BMP3, BMP4, BMP5, BMP6, BMP7, BMP8a, BMP8b, bone morphogenetic protein 1 (BMP1), CCL1/TCA3, CCL11, CCL12/MCP-5,CCL13/MCP-4, CCL14, CCL15, CCL16, CCL17/TARC, CCL18, CCL19, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CD153/CD30L/TNFSF8, CD40L/CD154/TNFSF5, CD40LG, CD70, CD70/CD27L/TNFSF7, CLCF1, c-MPL/CD110/ TPOR, CNTF, CX3CL1, CXCL1, CXCL10, CXCL11, CXCL12, CXCL13, CXCL14, CXCL15, CXCL16, CXCL17, CXCL2/MIP-2, CXCL3, CXCL4, CXCL5, CXCL6, CXCL7/Ppbp, CXCL9, EDA-A1, FAM19A1, FAM19A2, FAM19A3, FAM19A4, FAM19A5, Fas Ligand/FASLG/CD95L/CD178, GDF10, GDF11, GDF15, GDF2, GDF3, GDF4, GDF5, GDF6, GDF7, GDF8, GDF9, glial cell line-derived neurotrophic factor (GDNF), growth differentiation factor 1 (GDF1), IFNA1, IFNA10, IFNA13, IFNA14, IFNA2, IFNA4, IFNA5/IFNaG, IFNA7, IFNA8, IFNB1, IFNE, IFNG, IFNZ, IFNω/IFNW1, IL11, IL18, IL18BP, IL1A, IL1B, IL1F10, IL1F3/IL1RA, IL1F5, IL1F6, IL1F7, IL1F8, IL1F9, IL1RL2, IL31, IL33, IL6, IL8/CXCL8, inhibin-A, inhibin-B, Leptin, LIF, LTA/TNFB/TNFSF1, LTB/TNFC, neurturin (NRTN), OSM, OX-40L/TNFSF4/CD252, persephin (PSPN), RANKL/OPGL/TNFSF11(CD254), TL1A/TNFSF15, TNFA, TNF-alpha/TNFA, TNFSF10/TRAIL/APO-2L(CD253), TNFSF12, TNFSF13, TNFSF14/LIGHT/CD258, XCL1, and XCL2. In some embodiments, the target gene encodes for an immune checkpoint inhibitor. Non-limiting examples of such immune checkpoint inhibitors include PD-1, CTLA-4, LAG3, TIM-3, A2AR, B7-H3, B7-H4, BTLA, IDO, KIR, and VISTA. In some embodiments, the target gene encodes for a T cell receptor (TCR) alpha, beta, gamma, and/or delta chain.

A subject system can be introduced in a variety of immune cells, including any cell that is involved in an immune response. In some embodiments, immune cells comprise granulocytes such as asophils, eosinophils, and neutrophils; mast cells; monocytes which can develop into macrophages; antigen-presenting cells such as dendritic cells; and lymphocytes such as natural killer cells (NK cells), B cells, and T cells. In some embodiments, an immune cell is an immune effector cell. An immune effector cell refers to an immune cell that can perform a specific function in response to a stimulus. In some embodiments, an immune cell is an immune effector cell which can induce cell death. In some embodiments, the immune cell is a lymphocyte. In some embodiments, the lymphocyte is a NK cell. In some embodiments the lymphocyte is a T cell. In some embodiments, the T cell is an activated T cell. T cells include both naive and memory cells (e.g. central memory or T_{CM}, effector memory or T_{EM} and effector memory RA or T_{EMRA}), effector cells (e.g. cytotoxic T cells or CTLs or Tc cells), helper cells (e.g. Thl, Th2, Th3, Th9, Th7, TFH), regulatory cells (e.g. Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage. T cells can be divided into two broad categories: CD8⁺ T cells and CD4⁺ T cells, based on which protein is present on the cell’s surface. T cells expressing a subject system can carry out multiple functions, including killing infected cells and activating or recruiting other immune cells. CD8⁺ T cells are referred to as cytotoxic T cells or cytotoxic T lymphocytes (CTLs). CTLs expressing a subject system can be involved in recognizing and removing virus-infected cells and cancer cells. CTLs have specialized compartments, or granules, containing cytotoxins that cause apoptosis, e.g., programmed cell death. CD4⁺ T cells can be subdivided into four sub-sets - Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th17 cells are important for recruiting neutrophils.

A variety of cells can be used as a host cell to realize the systems and methods of the subject disclosure. A host cell to which any of the embodiments (e.g., a cell comprising or expressing the γδ TCR complex) disclosed herein can be applied (e.g., transduced) includes a wide variety of cell types. A host cell can be in vitro. A host cell can be in vivo. A host cell can be ex vivo. A host cell can be an isolated cell. A host cell can be a cell inside of an organism. A host cell can be an organism. A host cell can be a cell in a cell culture. A host cell can be one of a collection of cells. A host cell can be a mammalian cell or derived from a mammalian cell. A host cell can be a rodent cell or derived from a rodent cell. A host cell can be a human cell or derived from a human cell. A host cell can be a prokaryotic cell or derived from a prokaryotic cell. A host cell can be a bacterial cell or can be derived from a bacterial cell. A host cell can be an archaeal cell or derived from an archaeal cell. A host cell can be a eukaryotic cell or derived from a eukaryotic cell. A host cell can be a pluripotent stem cell. A host cell can be a plant cell or derived from a plant cell. A host cell can be an animal cell or derived from an animal cell. A host cell can be an invertebrate cell or derived from an invertebrate cell. A host cell can be a vertebrate cell or derived from a vertebrate cell. A host cell can be a microbe cell or derived from a microbe cell. A host cell can be a fungi cell or derived from a fungi cell. A host cell can be from a specific organ or tissue.

A host cell can be an immune cell, as abovementioned in the subject disclosure.

A host cell can be a stem cell or progenitor cell. Host cells can include stem cells (e.g., adult stem cells, embryonic stem cells, induced pluripotent stem (iPS) cells) and progenitor cells (e.g., cardiac progenitor cells, neural progenitor cells, etc.). Host cells can include mammalian stem cells and progenitor cells, including rodent stem cells, rodent progenitor cells, human stem cells, human progenitor cells, etc. Clonal cells can comprise the progeny of a cell. A host cell can be in a living organism. A host cell can be a genetically modified cell.

A host cell can be a totipotent stem cell, however, in some embodiments of this disclosure, the term “cell” may be used but may not refer to a totipotent stem cell. A host cell can be a plant cell, but in some embodiments of this disclosure, the term “cell” may be used but may not refer to a plant cell. A host cell can be a pluripotent cell. For example, a host cell can be a pluripotent hematopoietic cell that can differentiate into other cells in the hematopoietic cell lineage but may not be able to differentiate into any other non-hematopoietic cell. A host cell may be able to develop into a whole organism. A host cell may or may not be able to develop into a whole organism. A host cell may be a whole organism.

A variety of one or more intrinsic signaling pathways (e.g. NFkB) of a cell are available for embodiments provided herein. Table 1 provides exemplary signaling pathways and genes associated with the signaling pathway. A signaling pathway activated by stimulant binding to a cell (e.g., an immune cell, a stem cell, etc.) and/or a ligand binding to a transmembrane receptor in embodiments provided herein can be any one of those provided in Table 1. In an example, a promoter activated to drive expression of the GMP upon binding of a stimulant to the stimulant binding domain of a transmembrane receptor in embodiments provided can comprise the promoter sequence driving any of the genes provided in Table 1, any variant of the promoter sequence, or any partial promoter sequence (e.g., a minimal promoter sequence).

TABLE 1

| | |
|---|---|
| CELLULAR FUNCTION | GENES |
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor Signaling | RAC1; TAF4B; EP300; SMAD2; TRAF6; PCAF; ELK1; |
| | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; E1F4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4, AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton Signaling | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington’s Disease Signaling | PRKCE; IGF1; EP300; RCOR1; PRKCZ; HDAC4; TGM2; |
| | MAPK1; CAPNS1; AKT2; EGFR; NCOR2; SP1; CAPN2; |
| | PIK3CA; HDAC5; CREB1; PRKC1; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1;NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation Signaling | ACTN4; CD44; PRKCE; ITGAM; ROCK1; CXCR4; CYBA; |
| | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response Signaling | IRAK1; SOD2; MYD88; TRAF6; ELK1; MAPK1; PTPN11; |
| | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1;NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor Signaling | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism Signaling | PRKCE; EP300; PRKCZ; RXRA; MAPK1; NQO1; |
| | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAR/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | RNCOA3; MAPK14; INSR; RAF1; IKBKG; RELB; MAP3K7; |
| | CREBBP; MAP2K2; JAK2; CHUK; MAP2K1;NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ: TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4: PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5: PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin Signaling | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2: ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2: MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-Mediated Oxidative Stress Response | PRKCE; EP300; SOD2; PRKCZ; MAPK1; SQSTM1; |
| | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic Stellate Cell Activation | EDN1; IGF1; KDR; FLT1; SMAD2; FGFR1; MET; PGF; |
| | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled Receptor Signaling | PRKCE; RAP1A; RGS16; MAPK1; GNAS; AKT2; IKBKB; |
| | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1;NFKB1; BRAF; ATF4; AKT3; |
| | PRKCA |
| Inositol Phosphate Metabolism | PRKCE; IRAK1; PRKAA2; EIF2AK2; PTEN; GRK6; |
| | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S Checkpoint Regulation | HDAC4; SMAD3; SUV39H1; HDAC5; CDKN1B; BTRC; |
| | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA, PIK3C2A; BTK; LCK; RAF1; IKBKG; RELB, FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral Sclerosis Signaling | BID; IGF1; RAC1; BIRC4; PGF; CAPNS1; CAPN2; |
| | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide Metabolism | PRKCE; IRAK1; PRKAA2; EIF2AK2; GRK6; MAPK1; |
| | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term Depression | PRKCE; IGF1; PRKCZ; PRDX6; LYN; MAPK1; GNAS; |
| | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor Signaling | TAF4B; EP300; CARM1; PCAF; MAPK1; NCOR2; |
| | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination Pathway | TRAF6; SMURF1; BIRC4; BRCA1; UCHL1; NEDD4; |
| | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKC1; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term Potentiation | PRKCE; RAP1A; EP300; PRKCZ; MAPK1; CREB1; |
| | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the Cardiovascular System | EDN1; PTEN; EP300; NQO1; UBE2I; CREB1; ARNT; |
| | HIF1A; SLC2A4; NOS3; TP53; LDHA; AKT1; ATM; |
| | VEGFA; JUN; ATF4; VHL; HSP90AA1 |
| LPS/IL-1 Mediated Inhibition of RXR Function | IRAK1; MYD88; TRAF6; PPARA; RXRA; ABCA1, |
| | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA Damage Checkpoint Regulation | EP300; PCAF; BRCA1; GADD45A; PLK1; BTRC; |
| | CHEK1; ATR; CHEK2; YWHAZ; TP53; CDKN1A; |
| | PRKDC; ATM; SFN; CDKN2A |
| | KDR; FLT1; PGF; AKT2; PIK3CA; PIK3CB; PIK3C3; |
| Nitric Oxide Signaling in the Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1;BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum Stress Pathway | HSPA5; MAPK8; XBP1; TRAF2; ATF6; CASP9; ATF4; |
| | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson’s Signaling | UCHL1; MAPK8; MAPK13; MAPK14; CASP9; PARK7; |
| | PARK2; CASP3 |
| Cardiac & Beta Adrenergic Signaling | GNAS; GNAQ; PPP2R1A; GNB2L1; PPP2CA; PPP1CC; |
| | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRKIB |
| Glycerophospholipid Metabolism | PLD1; GRN; GPAM; YWHAZ; SPHK1; SPHK2 |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair Pathway | ERCC5; ERCC4; XPA; XPC; ERCC1 |
| Starch and Sucrose Metabolism | UCHL1; HK2; GCK; GPI; HK 1 |
| Aminosugars Metabolism | NQO1; HK2; GCK; HK1 |
| Arachidonic Acid Metabolism | PRDX6; GRN; YWHAZ; CYP1B1 |
| Circadian Rhythm Signaling | CSNK1E; CREB1; ATF4; NR1D1 |
| Coagulation System | BDKRB1; F2R; SERPINE1; F3 |
| Dopamine Receptor Signaling | PPP2R1A; PPP2CA; PPP1CC; PPP2R5C |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline Metabolism | ALDH1A1; NOS3; NOS2A |
| Eicosanoid Signaling | PRDX6; GRN; YWHAZ |
| Fructose and Mannose Metabolism | HK2; GCK; HK1 |
| Galactose Metabolism | HK2; GCK; HK1 |
| Stilbene, Coumarine and Lignin Biosynthesis | PRDX6; PRDX1; TYR |
| Antigen Presentation Pathway | CALR; B2M |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid Metabolism | PRDX6; CHKA |
| Histidine Metabolism | PRMT5; ALDH1A1 |
| Inositol Metabolism | ERO1L; APEX1 |
| Metabolism of Xenobiotics by Cytochrome p450 | GSTP1; CYP1B1 |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid Metabolism | PRMT5; AHCY |
| Sphingolipid Metabolism | SPHK1; SPHK2 |
| Aminophosphonate Metabolism | PRMT5 |
| Androgen and Estrogen Metabolism | PRMT5 |
| Ascorbate and Aldarate Metabolism | ALDH1A1 |
| Bile Acid Biosynthesis | ALDH1A1 |
| Cysteine Metabolism | LDHA |
| Fatty Acid Biosynthesis | FASN |
| Glutamate Receptor Signaling | GNB2L1 |
| NRF2-mediated Oxidative Stress Response | PRDX1 |
| Pentose Phosphate Pathway | GPI |
| Pentose and Glucuronate Interconversions | UCHL1 |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and Isoleucine Degradation | ALDH1A1 |
| Glycine, Serine and Threonine Metabolism | CHKA |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4fl or Brn3a); Numb; Reln |

### Therapeutic Use(s)

Systems and compositions of the present disclosure are useful for a variety of applications. For example, systems and methods of the present disclosure are useful in methods of regulating gene expression and/or cellular activity. In an aspect, the systems and compositions disclosed herein are utilized in methods of regulating gene expression and/or cellular activity in an immune cell. Immune cells regulated using a subject system can be useful in a variety of applications, including, but not limited to, immunotherapy to treat diseases and disorders. Diseases and disorders that can be treated using modified immune cells of the present disclosure include inflammatory conditions, cancer, and infectious diseases. In some embodiments, immunotherapy is used to treat cancer.

In some cases, regulating the expression of the target polynucleotide in the cell may enhance and/or prolong cytotoxicity of the cell against the tumor cell or cancer cell, as compared to without the regulating (or without binding of the ligand to the chimeric receptor). In some cases, regulating the expression of the target polynucleotide in the cell may be evidenced by at least about 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 300 fold, 400 fold, 500 fold, or greater cytotoxicity against the tumor/cancer cell, as compared to without the regulating (or without binding of the ligand to the chimeric receptor). In some cases, regulating the expression of the target polynucleotide in the cell may be evidenced by prolonging cytotoxicity of the cell against the tumor/cancer cell by at least about 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 300 fold, 400 fold, 500 fold, or longer, as compared to without the regulating (or without binding of the ligand to the chimeric receptor).

In some cases, regulating the expression of the target polynucleotide in the cell may reduce a size of a tumor as compared to without the regulating (or without binding of the ligand to the chimeric receptor), or obliterates the tumor. In some cases, regulating the expression of the target polynucleotide in the cell may be evidenced by at least about 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 300 fold, 400 fold, 500 fold, or greater reduction in the size of the tumor, as compared to without the regulating (or without binding of the ligand to the chimeric receptor).

In some cases, regulating the expression of the target polynucleotide in the cell may increase expression of one or more cytokines and/or one or more cell surface receptors by the cell, as compared to without the regulating (or without binding of the ligand to the chimeric receptor). In some cases, regulating the expression of the target polynucleotide in the cell may be evidenced by at least about 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 300 fold, 400 fold, 500 fold, or greater expression of the one or more cytokines and/or the one or more cell surface receptors by the cell, as compared to without the regulating (or without binding of the ligand to the chimeric receptor).

In some cases, regulating the expression of the target polynucleotide in the cell may decrease expression of one or more cytokines and/or one or more cell surface receptors by the cell, as compared to without the regulating (or without binding of the ligand to the chimeric receptor). In some cases, regulating the expression of the target polynucleotide in the cell may be evidenced by at least about 2 fold, 3 fold, 4 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, 100 fold, 150 fold, 200 fold, 300 fold, 400 fold, 500 fold, or greater reduction in the expression of the one or more cytokines and/or the one or more cell surface receptors by the cell, as compared to without the regulating (or without binding of the ligand to the chimeric receptor).

A variety of target cells can be killed using the systems and methods of the subject disclosure. A target cell to which this method can be applied includes a wide variety of cell types. A target cell can be in vitro. A target cell can be in vivo. A target cell can be ex vivo. A target cell can be an isolated cell. A target cell can be a cell inside of an organism. A target cell can be an organism. A target cell can be a cell in a cell culture. A target cell can be one of a collection of cells. A target cell can be a mammalian cell or derived from a mammalian cell. A target cell can be a rodent cell or derived from a rodent cell. A target cell can be a human cell or derived from a human cell. A target cell can be a prokaryotic cell or derived from a prokaryotic cell. A target cell can be a bacterial cell or can be derived from a bacterial cell. A target cell can be an archaeal cell or derived from an archaeal cell. A target cell can be a eukaryotic cell or derived from a eukaryotic cell. A target cell can be a pluripotent stem cell. A target cell can be a plant cell or derived from a plant cell. A target cell can be an animal cell or derived from an animal cell. A target cell can be an invertebrate cell or derived from an invertebrate cell. A target cell can be a vertebrate cell or derived from a vertebrate cell. A target cell can be a microbe cell or derived from a microbe cell. A target cell can be a fungi cell or derived from a fungi cell. A target cell can be from a specific organ or tissue.

A target cell can be a stem cell or progenitor cell. Target cells can include stem cells (e.g., adult stem cells, embryonic stem cells, induced pluripotent stem (iPS) cells) and progenitor cells (e.g., cardiac progenitor cells, neural progenitor cells, etc.). Target cells can include mammalian stem cells and progenitor cells, including rodent stem cells, rodent progenitor cells, human stem cells, human progenitor cells, etc. Clonal cells can comprise the progeny of a cell. A target cell can comprise a target nucleic acid. A target cell can be in a living organism. A target cell can be a genetically modified cell. A target cell can be a host cell.

A target cell can be a totipotent stem cell, however, in some embodiments of this disclosure, the term “cell” may be used but may not refer to a totipotent stem cell. A target cell can be a plant cell, but in some embodiments of this disclosure, the term “cell” may be used but may not refer to a plant cell. A target cell can be a pluripotent cell. For example, a target cell can be a pluripotent hematopoietic cell that can differentiate into other cells in the hematopoietic cell lineage but may not be able to differentiate into any other non-hematopoietic cell. A target cell may be able to develop into a whole organism. A target cell may or may not be able to develop into a whole organism. A target cell may be a whole organism.

A target cell can be a primary cell. For example, cultures of primary cells can be passaged 0 times, 1 time, 2 times, 4 times, 5 times, 10 times, 15 times or more. Cells can be unicellular organisms. Cells can be grown in culture.

A target cell can be a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

If the target cells are primary cells, they may be harvested from an individual by any method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank’s balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

Non-limiting examples of cells which can be target cells include, but are not limited to, lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph ); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley’s layer, Hair root sheath cell of Henle’s layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme -rich secretion), Von Ebner’s gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex -hormone sensitive), Gland of Moll cell in eyelid (specialized sweat gland), Sebaceous gland cell (lipid-rich sebum secretion), Bowman’s gland cell in nose (washes olfactory epithelium), Brunner’s gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin’s gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Of particular interest are cancer cells. In some embodiments, the target cell is a cancer cell. Non-limiting examples of cancer cells include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt’s lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman’s Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing’s sarcoma, Extracranial Germ Cell Tumor, Extragonadal Germ Cell Tumor, Extrahepatic Bile Duct Cancer, Extramammary Paget’s disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin’s lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi’s sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget’s disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter’s transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom’s macroglobulinemia, Warthin’s tumor, Wilms’ tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

In some embodiments, the target cells form a tumor. A tumor treated with the methods herein can result in stabilized tumor growth (e.g., one or more tumors do not increase more than 1%, 5%, 10%, 15%, or 20% in size, and/or do not metastasize). In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

Death of target cells can be determined by any suitable method, including, but not limited to, counting cells before and after treatment, or measuring the level of a marker associated with live or dead cells (e.g. live or dead target cells). Degree of cell death can be determined by any suitable method. In some embodiments, degree of cell death is determined with respect to a starting condition. For example, an individual can have a known starting amount of target cells, such as a starting cell mass of known size or circulating target cells at a known concentration. In such cases, degree of cell death can be expressed as a ratio of surviving cells after treatment to the starting cell population. In some embodiments, degree of cell death can be determined by a suitable cell death assay. A variety of cell death assays are available, and can utilize a variety of detection methodologies. Examples of detection methodologies include, without limitation, the use of cell staining, microscopy, flow cytometry, cell sorting, and combinations of these.

When a tumor is subject to surgical resection following completion of a therapeutic period, the efficacy of treatment in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%). In some embodiments, the necrosis percentage of the resected tissue is 100%, that is, no living tumor tissue is present or detectable.

In some cases, exposing a target cell to an immune cell or population of immune cells disclosed herein can be conducted either in vitro or in vivo. Exposing a target cell to an immune cell or population of immune cells generally refers to bringing the target cell in contact with the immune cell and/or in sufficient proximity such that an antigen of a target cell (e.g., membrane bound or non-membrane bound) can bind to the ligand interacting domain of the chimeric transmembrane receptor polypeptide expressed in the immune cell. Exposing a target cell to an immune cell or population of immune cells in vitro can be accomplished by co-culturing the target cells and the immune cells. Target cells and immune cells can be co-cultured, for example, as adherent cells or alternatively in suspension. Target cells and immune cells can be co-cultured in various suitable types of cell culture media, for example with supplements, growth factors, ions, etc. Exposing a target cell to an immune cell or population of immune cells in vivo can be accomplished, in some cases, by administering the immune cells to a subject, for example a human subject, and allowing the immune cells to localize to the target cell via the circulatory system. In some cases, an immune cell can be delivered to the immediate area where a target cell is localized, for example, by direct injection.

Exposing can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In some embodiments, cells expressing a system provided herein induce death of a target cell in an in vitro cell death assay. The cells expressing a system provided herein may exhibit enhanced ability to induce death of the target cell compared to control cells not expressing a system of the present disclosure. In some cases, the enhanced ability to induce death of the target cell is at least a 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3.0-fold, 3.5-fold, 4.0-fold, 5-fold, 10-fold, 100-fold, or 1000-fold increase in induced cell death. The degree of induced cell death can be determined at any suitable time point, for example, at least 4 hours, 6 hours, 8 hours, 12 hours, 16 hours, 24 hours, 36 hours, 48 hours, or 52 hours after contacting the cell to the target cell.

In some embodiments, a target polynucleotide can comprise one or more disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissue compared with tissue(s) or cells of a non-disease control. In some embodiments, it is a gene that becomes expressed at an abnormally high level. In some embodiments, it is a gene that becomes expressed at an abnormally low level. The altered expression can correlate with the occurrence and/or progression of the disease. A disease-associated gene also refers to a gene possessing mutation(s) or genetic variation that is directly responsible or is in linkage disequilibrium with a gene(s) that is response for the etiology of a disease. The transcribed or translated products may be known or unknown, and may be at a normal or abnormal level.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web. Exemplary genes associated with certain diseases and disorders are provided in Tables 2 and 3.

Mutations in these genes and pathways can result in production of improper proteins or proteins in improper amounts which affect function.

Promoters that can be used with the methods and compositions of the disclosure include, for example, promoters active in a eukaryotic, mammalian, non-human mammalian or human cell. The promoter can be an inducible or constitutively active promoter. Alternatively or additionally, the promoter can be tissue or cell specific. The promoter can be native or composite promoter.

Non-limiting examples of suitable eukaryotic promoters (i.e. promoters functional in a eukaryotic cell) can include those from cytomegalovirus (CMV) immediate early, herpes simplex virus (HSV) thymidine kinase, early and late SV40, long terminal repeats (LTRs) from retrovirus, human elongation factor-1 promoter (EF1), ubiquitin B promoter (UB), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter can be cell, tissue or tumor specific, such as CD45 promoter, AFP promoter, human Albumin promoter (Alb), MUC 1 promoter, COX2 promoter, SP-B promoter, OG-2 promoter. The promoter can be a fungi promoter. The promoter can be a plant promoter. A database of plant promoters can be found (e.g., PlantProm). The expression vector may also contain a ribosome binding site for translation initiation and a transcription terminator. The expression vector may also include appropriate sequences for amplifying expression. Another example of a promoter for the expression vector may include myeloproliferative sarcoma virus enhancer, negative control region deleted, dl587rev primer-binding site substituted (MND) promoter. A promoter for driving RNA can include RNA Pol III promoters (e.g., U6 or H1), Pol II promoters, and/or tRNA(val) promoter.

TABLE 2

| | |
|---|---|
| DISEASE/DISORDERS | GENE(S) |
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bc12; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular Degeneration | Abcr; Ccl2; Cc2; cp (ceruloplasmin); Timp3; cathepsinD; |
| | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide Repeat Disorders | HTT (Huntington’s Dx); SBMA/SMAX1/AR (Kennedy’s |
| | Dx); FXN/X25 (Friedrich’s Ataxia); ATX3 (Machado- |
| | Joseph’s Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer’s); Atxn7; Atxn10 |
| Fragile X Syndrome | FMR2; FXR1; FXR2; mGLUR5 |
| Secretase Related Disorders | APH-1 (alpha and beta); Presenilin (Psen1); nicastrin |
| | (Ncstn); PEN-2 |
| Others | Nos1; Parp1; Nat1; Nat2 |
| Prion - related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1(alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer’s Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson’s Disease | x-Synuclein; DJ-1; LRRK2; Parkin; PINK1 |

TABLE 3

| | |
|---|---|
| Blood and coagulation | Anemia (CDAN1, CDA1, RPS19, DBA, PKLR, PK1, NT5C3, UMPH1, |
| | PSN1, RHAG, RH50A, NRAMP2, SPTB, ALAS2, ANH1, ASB, |
| | ABCB7, ABC7, ASAT); Bare lymphocyte syndrome (TAPBP, TPSN, |
| diseases and disorders | TAP2, ABCB3, PSF2, RING11, MHC2TA, C2TA, RFX5, RFXAP, |
| | RFX5), Bleeding disorders (TBXA2R, P2RX1, P2X1); Factor H and |
| | factor H-like 1 (HF1, CFH, HUS); Factor V and factor VIII (MCFD2); |
| | Factor VII deficiency (F7); Factor X deficiency (F10); Factor XI |
| | deficiency (F11); Factor XII deficiency (F12, HAF); Factor XIIIA |
| | deficiency (F13A1, F13A); Factor XIIIB deficiency (F13B); Fanconi |
| | anemia (FANCA, FACA, FA1, FA, FAA, FAAP95, FAAP90, FLJ34064, |
| | FANCB, FANCC, FACC, BRCA2, FANCD1, FANCD2, FANCD, |
| | FACD, FAD, FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A (F8, F8C, |
| | HEMA); Hemophilia B (F9, HENB), Hemorrhagic disorders (PI, ATT, |
| | F5); Leukocyde deficiencies and disorders (ITGB2, CD18, LCAMB, |
| | LAD, EIF2B1, EIF2BA, EIF2B2, EIF2B3, EIF2B5, LVWM, CACH, |
| | CLE, EIF2B4); Sickle cell anemia (HBB); Thalassemia (HBA2, HBB, |
| | HBD, LCRB, HBA1). |
| Cell dysregulation and oncology diseases and disorders | B-cell non-Hodgkin lymphoma (BCL7A, BCL7); Leukemia (TAL1 |
| | TCL5, SCL, TAL2, FLT3, NBS1, NBS, ZNFN1A1, IK1, LYF1, |
| | HOXD4, HOX4B, BCR, CML, PHL, ALL, ARNT, KRAS2, RASK2, |
| | GMPS, AF10, ARHGEF12, LARG, KIAA0382, CALM, CLTH, |
| | CEBPA, CEBP, CHIC2, BTL, FLT3, KIT, PBT, LPP, NPM1, NUP214, |
| | D9S46E, CAN, CAIN, RUNX1, CBFA2, AML1, WHSC1L1, NSD3, |
| | FLT3, AF1Q, NPM1, NUMA1, ZNF145, PLZF, PML, MYL, STAT5B, |
| | AF10, CALM, CLTH, ARL11, ARLTS1, P2RX7, P2X7, BCR, CML, |
| | PHL, ALL, GRAF, NF1, VRNF, WSS, NFNS, PTPN11, PTP2C, SHP2, |
| | NS1, BCL2, CCND1, PRAD1, BCL1, TCRA, GATA1, GF1, ERYF1, |
| | NFE1, ABL1, NQO1, DIA4, NMOR1, NUP214, D9S46E, CAN, CAIN). |
| Inflammation and immune related diseases and disorders | AIDS (KIR3DL1, NKAT3, NKB1, AMB11, KIR3DS1, IFNG, CXCL12, |
| | SDF1); Autoimmune lymphoproliferative syndrome (TNFRSF6, APT1, |
| | FAS, CD95, ALPS1A); Combined immunodeficiency, (IL2RG, |
| | SCIDX1, SCIDX, IMD4); HIV-1 (CCL5, SCYA5, D17S136E, TCP228), |
| | HIV susceptibility or infection (IL10, CSIF, CMKBR2, CCR2, |
| | CMKBR5, CCCKR5 (CCR5)); Immunodeficiencies (CD3E, CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, PIDX, |
| | TNFRSF14B, TACI); Inflammation (IL-10, IL-1 (IL-1a, IL-1b), IL-13, |
| | IL-17 (IL-17a (CTLA8), IL-17b, IL-17c, IL-17d, IL-17f), 11-23, Cx3cr1, |
| | ptpn22, TNFa, NOD2/CARD15 for IBD, IL-6, IL-12 (IL-12a, IL-12b), |
| | CTLA4, Cx3cl1); Severe combined immunodeficiencies (SCIDs)(JAK3, |
| | JAKL, DCLREIC, ARTEMIS, SCIDA, RAG1, RAG2, ADA, PTPRC, |
| | CD45, LCA, IL7R, CD3D, T3D, IL2RG, SCIDX1, SCIDX, IMD4). |
| | Amyloid neuropathy (TTR, PALB); Amyloidosis (APOA1, APP, AAA, |
| Metabolic, liver, kidney and protein diseases and disorders | CVAP, AD1, GSN, FGA, LYZ, TTR, PALB); Cirrhosis (KRT18, KRT8, |
| | CIRH1A, NAIC, TEX292, KIAA1988); Cystic fibrosis (CFTR, ABCC7, |
| | CF, MRP7); Glycogen storage diseases (SLC2A2, GLUT2, G6PC, |
| | G6PT, G6PT1, GAA, LAMP2, LAMPB, AGL, GDE, GBE1, GYS2, |
| | PYGL, PFKM); Hepatic adenoma, 142330 (TCF1, HNF1A, MODY3), |
| | Hepatic failure, early onset, and neurologic disorder (SCOD1, SCO1), |
| | Hepatic lipase deficiency (LTPC), Hepatoblastoma, cancer and |
| | carcinomas (CTNNB1, PDGFRL, PDGRL, PRLTS, AXIN1, AXIN, |
| | CTNNB1, TP53, P53, LFS1, IGF2R, MPRI, MET, CASP8, MCH5; |
| | Medullary cystic kidney disease (UMOD, HNFJ, FJHN, MCKD2, |
| | ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/Skeletal diseases and disorders | Becker muscular dystrophy (DMD, BMD, MYF6), Duchenne Muscular |
| | Dystrophy (DMD, BMD);Emery-Dreifuss muscular dystrophy (LMNA, |
| | LMN1, EMD2, FPLD, CMD1A, HGPS, LGMD1B, LMNA, LMN1, |
| | EMD2, FPLD, CMD1A); Facioscapulohumeral muscular dystrophy |
| | (FSHMD1A, FSHD1A); Muscular dystrophy (FKRP, MDC1C, |
| | LGMD2I, LAMA2, LAMM, LARGE, KIAA0609, MDC1D, FCMD, |
| | TTID, MYOT, CAPN3, CANP3, DYSF, LGMD2B, SGCG, LGMD2C, |
| | DMDA1, SCG3, SGCA, ADL, DAG2, LGMD2D, DMDA2, SGCB, |
| | LGMD2E, SGCD, SGD, LGMD2F, CMD1L, TCAP, LGMD2G, |
| | CMD1N, TRIM32, HT2A, LGMD2H, FKRP, MDC1C, LGMD2I, TTN, |
| | CMD1G, TMD, LGMD2J, POMT1, CAV3, LGMD1C, SEPN1, SELN, |
| | RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, |
| | TIRC7, OC116, OPTB1); Muscular atrophy (VAPB, VAPC, ALS8, |
| | SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological and neuronal diseases and disorders | ALS (SOD1, ALS2, STEX, FUS, TARDBP, VEGF (VEGF-a, VEGF-b, |
| | VEGF-c); Alzheimer disease (APP, AAA, CVAP, AD1, APOE, AD2, |
| | PSEN2, AD4, STM2, APBB2, FE65L1, NOS3, PLAU, URK, ACE, |
| | DCP1, ACE1, MPO, PACIP1, PAXIP1L, PTIP, A2M, BLMH, BMH, |
| | PSEN1, AD3); Autism (Mecp2, BZRAP1, MDGA2, Sema5A, Neurexin |
| | 1, GLO1, MECP2, RTT, PPMX, MRX16, MRX79, NLGN3, NLGN4, |
| | KIAA1260, AUTSX2); Fragile X Syndrome (FMR2, FXR1, FXR2, |
| | mGLUR5); Huntington’s disease and disease like disorders (HD, IT15, |
| | PRNP, PRIP, JPH3, JP3, HDL2, TBP, SCA17); Parkinson disease |
| | (NR4A2, NURR1, NOT, TINUR, SNCAIP, TBP, SCA17, SNCA, |
| | NACP, PARK1, PARK4, DJ1, PARK7, LRRK2, PARK8, PINK1, |
| | PARK6, UCHL1, PARK5, SNCA, NACP, PARK1, PARK4, PRKN, |
| | PARK2, PDJ, DBH, NDUFV2); Rett syndrome (MECP2, RTT, PPMX, |
| | MRX16, MRX79, CDKL5, STK9, MECP2, RTT, PPMX, MRX16, |
| | MRX79, x-Synuclein, DJ-1); Schizophrenia (Neuregulin1 (Nrg1), Erb4 |
| | (receptor for Neuregulin), Complexin1 (Cplx1), Tph1 Tryptophan |
| | hydroxylase, Tph2, Tryptophan hydroxylase 2, Neurexin 1, GSK3, |
| | GSK3a, GSK3b, 5-HTT (Slc6a4), COMT, DRD (Drd1a), SLC6A3, |
| | DAOA, DTNBP1, Dao (Dao1)); Secretase Related Disorders (APH-1 |
| | (alpha and beta), Presenilin (Psen1), nicastrin, (Ncstn), PEN-2, Nos1, |
| | Parp1, Natl, Nat2); Trinucleotide Repeat Disorders (HTT (Huntington’s |
| | Dx), SBMA/SMAX1/AR (Kennedy’s Dx), FXN/X25 (Friedrich’s |
| | Ataxia), ATX3 (Machado- Joseph’s Dx), ATXN1 and ATXN2 |
| | (spinocerebellar ataxias), DMPK (myotonic dystrophy), Atrophin-1 and |
| | Atn1 (DRPLA Dx), CBP (Creb-BP - global instability), VLDLR |
| | (Alzheimer’s), Atxn7, Atxn10). |
| Ocular diseases and disorders | Age-related macular degeneration (Abcr, Ccl2, Cc2, cp (ceruloplasmin), |
| | Timp3, cathepsinD, Vldlr, Ccr2); Cataract (CRYAA, CRYA1, CRYBB2, |
| | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, |
| | CRYGD, CRYG4, BFSP2, CP49, CP47, HSF4, CTM, HSF4, CTM, |
| | MIP, AQP0, CRYAB, CRYA2, CTPP2, CRYBB1, CRYGD, CRYG4, |
| | CRYBB2, CRYB2, CRYGC, CRYG3, CCL, CRYAA, CRYA1, GJA8, |
| | CX50, CAE1, GJA3, CX46, CZP3, CAE3, CCM1, CAM, KRIT1); |
| | Corneal clouding and dystrophy (APOA1, TGFBI, CSD2, CDGG1, |
| | CSD, BIGH3, CDG2, TACSTD2, TROP2, M1S1, VSX1, RINX, PPCD, |
| | PPD, KTCN, COL8A2, FECD, PPCD2, PIP5K3, CFD); Cornea plana |
| | congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, |
| | NTG, NPG, CYP1B1, GLC3A); Leber congenital amaurosis (CRB1, |
| | RP12, CRX, CORD2, CRD, RPGRIP1, LCA6, CORD9, RPE65, RP20, |
| | AIPL1, LCA4, GUCY2D, GUC2D, LCA1, CORD6, RDH12, LCA3); |
| | Macular dystrophy (ELOVL4, ADMD, STGD2, STGD3, RDS, RP7, |
| | PRPH2, PRPH, AVMD, AOFMD, VMD2). |

Systems and compositions of the present disclosure are useful for other varieties of applications. For example, systems and methods of the present disclosure are useful in methods of regulating gene expression and/or cellular activity critical for cell proliferation, differentiation, trans-differentiation, and/or de-differentiation during tissue (e.g., an organ) growth, repair, regeneration, regenerative medicine, and/or engineering. Examples of the tissue include epithelial, connective, nerve, muscle, organ, and other tissues. Other exemplary tissues include artery, ligament, skin, tendon, kidney, nerve, liver, pancreas, bladder, bone, lung, blood vessels, heart valve, cartilage, eyes, etc.

Systems and methods of the present disclosure may be combined with or modified by other systems and methods, such as, for example, those described in U.S. Pat. No. 9,856,497 (“CHIMERIC PROTEINS AND METHODS OF REGULATING GENE EXPRESSION”), Patent Cooperation Treaty Patent Publication No. 2017/123559 (“CHIMERIC PROTEINS AND METHODS OF REGULATING GENE EXPRESSION”), Patent Cooperation Treaty Patent Publication No. 2017/123556 (“CHIMERIC PROTEINS AND METHODS OF IMMUNOTHERAPY”), Patent Cooperation Treaty Patent Publication No. 2019/014390 (“METHODS AND SYSTEMS FOR CONDITIONALLY REGULATING GENE EXPRESSION”), and Patent Cooperation Treaty Patent Application No. PCT/US2019/023721 (“GENE REGULATION VIA CONDITIONAL NUCLEAR LOCALIZATION OF GENE MODULATING POLYPEPTIDES”), each of which is entirely incorporated herein by reference.

### EXAMPLES

Various aspects of the disclosure are further illustrated by the following non-limiting examples.

### Example 1: System and Methods for Regulating Signaling of a Receptor in a Cell

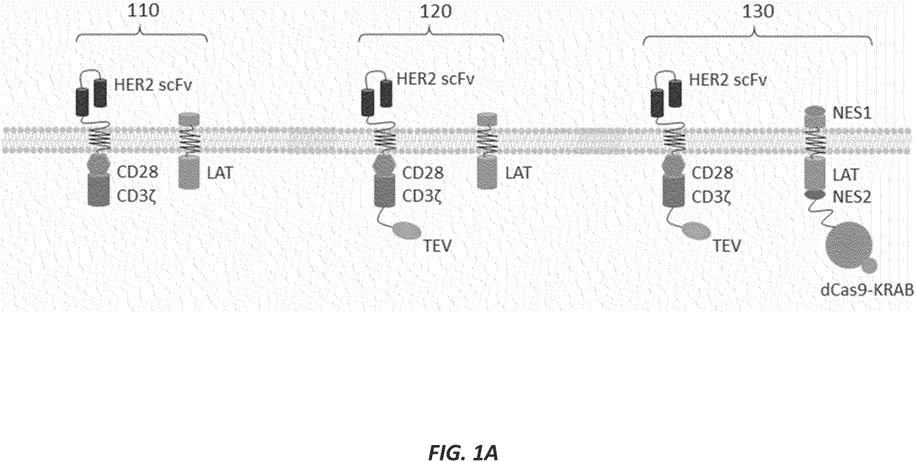

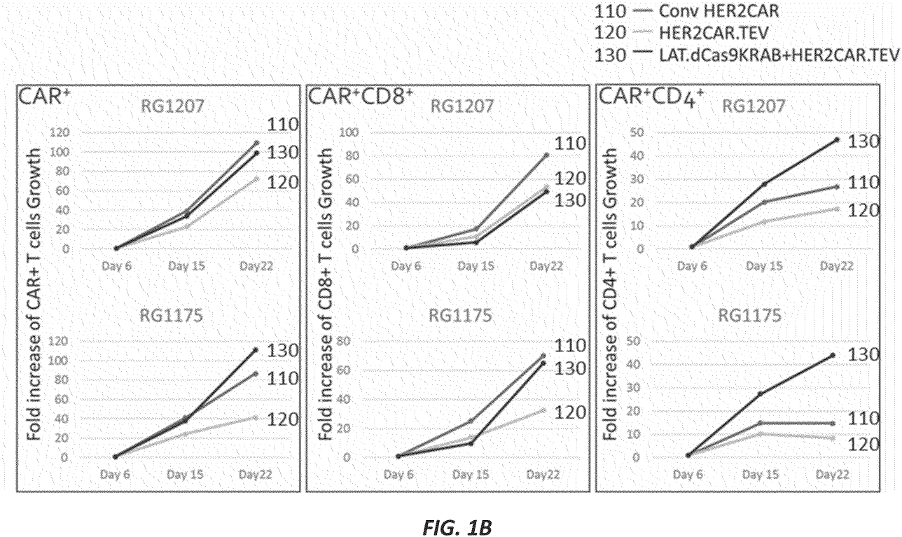

FIG. 1A schematically illustrates three example systems comprising a receptor (e.g., a chimeric receptor) and an adaptor protein of the receptor. The adaptor protein may be a wild-type adaptor protein. Alternatively, the adaptor protein may be a chimeric adaptor protein comprising at least one heterologous nuclear localization signal (NES) and/or a gene modulating polypeptide (GMP) comprising an actuator (e.g., dCase9-KRAB) capable of modulating expression of a target polynucleotide (e.g., gene) in a cell. System 110 (i.e., “Conventional HER2” or “Conv.HER2”) comprises a CAR that includes a ligand binding antibody (e.g., anti-HER2 scFv), a transmembrane domain, and an intracellular domain. The intracellular domain of the CAR comprises (i) at least a portion of a signaling domain of CD28 and/or (ii) at least a portion of a signaling domain of CD3zeta. The system 110 further comprises a wild-type adaptor protein of a cell (e.g., an adaptor protein of a T-cell receptor (TCR) of the cell, such as the linker for activation of T cell (LAT)). The wild-type adaptor protein may be endogenous or exogenous to the cell.

Referring to FIG. 1A, system 120 (i.e., “HER2.TEV”) comprises a modified version of the CAR of the system 110, wherein the CAR is further linked to a cleavage moiety (e.g., Tobacco Etch Virus (TEV) proteinase) capable of cleaving a target cleavage recognition site (e.g., TEV cleavage site (TCS)). The system 120 further comprises the native adaptor protein, as illustrated and described in the system 110. In this system, the cell may further express the GMP comprising the actuator moiety linked to the cleavage recognition site of the cleavage moiety.

Referring to FIG. 1A, system 130 (i.e. “NES1-LATb-NES2-TCS-dCas9-KRAB/HER2.TEV” or “LAT-dCas9-KRAB/HER2.TEV” or “LdCK/HER2.TEV”) comprises the CAR of the system 120. The system 130 further comprises a chimeric polypeptide comprising the adaptor protein. The adaptor protein is flanked by a first heterologous NES (i.e., NES1) and a second heterologous NES (i.e., NES2). The second heterologous NES is linked to the GMP comprising the actuator moiety linked to the cleavage recognition site of the cleavage moiety. Upon signaling of the CAR by contacting the CAR with a ligand (e.g., HER2), the chimeric polypeptide comprising the adaptor protein may be recruited towards the CAR, such that the cleavage moiety can cleave the cleavage recognition site, thereby releasing the actuator (e.g., dCas9KRAB) from the GMP of the chimeric polypeptide.

FIG. 1B schematically illustrates effect of T cell proliferation by the three systems 110, 120, and 130, as provided in FIG. 1A. The three systems 110, 120, and 130 may be transduced into primary human T cells. For each system, 110, 120, or 130, the receptor and the adaptor protein may be part of the same vector or different vectors. Each system, 110, 120, or 130, may be under the control of an endogenous and/or exogenous promoter of the host cell. The primary human T cells may be obtained from two donors, RG1207 and RG1175. The transduced primary human T cells may be cultured (e.g., in vitro) to allow cell proliferation. The transduced primary human T cells may be enriched (e.g., at day 6) using a cell sorter, and subsequently cultured. Expression of the respective receptor of the three systems (e.g., HER2 CAR of system 110 and HER2 CAR-TEV of systems 120 and 130) and/or one or more markers of the primary human T cells (e.g., CD4 and/or CD8) may be analyzed overtime (e.g., at least for 9 days, at least for 16 days) as marker(s) indicative or cell number or proliferation. Proliferation of the engineered primary human T cell may be calculated and represented as a fold increase of the expression level of the marker(s) with respect to that of the respective marker(s) at day 6.

Referring to FIG. 1B, the presence of the at least one heterologous NES in the adaptor protein of the system 130 may enhance proliferation of the host cell, in comparison to the system 110 and/or the system 120. In some cases, with CAR expression as a marker for cell proliferation, the system 130 may enhance proliferation of the primary human T cell (e.g. from RG1207) by at least about 30% in comparison to the system 120. With CAR expression as a marker for cell proliferation, the system 130 may enhance proliferation of the primary human T cell (e.g. from RG1175) by at least about 170% in comparison to the system 120, and by at least about 30% in comparison to the system 110. In some cases, with CAR and CD8 expressions as markers for cell proliferation, the system 130 may enhance proliferation of the primary human T cell (e.g. from RG1175) by at least about 100% in comparison to the system 110. In some cases, with CAR and CD4 expressions as markers for cell proliferation, the system 130 may enhance proliferation of the primary human T cell (e.g. from RG1207) by at least about 140% in comparison to the system 120, and by at least about 80% in comparison to the system 110. With CAR and CD4 expressions as markers for cell proliferation, the system 130 may enhance proliferation of the primary human T cell (e.g. from RG1175) by at least about 300% in comparison to the system 120, and by at least about 180% in comparison to the system 110.

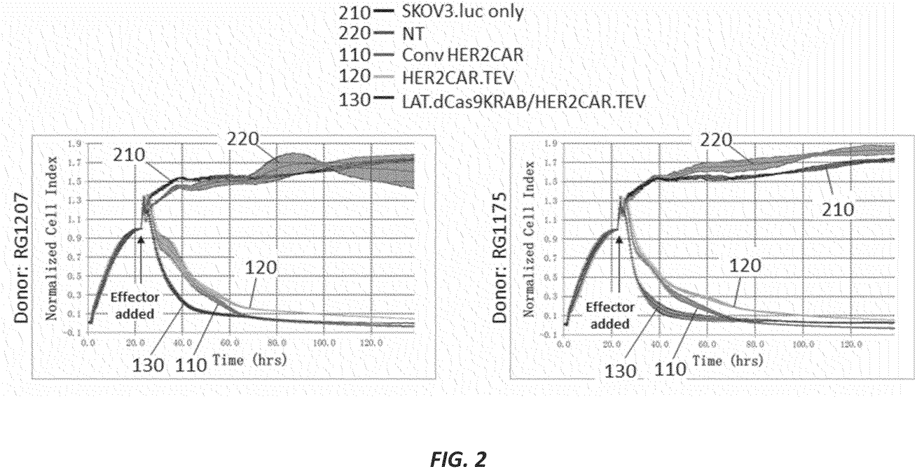

FIG. 2 schematically illustrates effect on T cells’ cytotoxicity against target cells (e.g., tumor cells, such as ovarian tumor cells) by the three systems 110, 120, and 130, as provided in FIG. 1A. The host primary human T cells may be provided and engineered, as provided and illustrated in FIG. 1B. The effector cells (e.g., T cells transduced with one of the three systems 110, 120, and 130, or control cells) may be cultured with the target cells (e.g., ovarian tumor cells, SKOV3, that carry a luciferase reporter, i.e., “SKOV3.luc”) at an effector-to-target (E:T) ratio (e.g., 1:5, 1:10, or 1:15), and cultured over the course of multiple days (e.g., 5 days). In some cases, the ovarian tumor cells may be seeded (e.g., in a 96-well Xcelligence tissue culture plate), and subsequently (e.g., 24 hours later), the control non-transduced (NT) T cells or the T cells transduced with one of the three systems 110, 120, and 130 may be co-cultured at 1:5 E:T ratio for 5 days. Kinetics (e.g., growth or metabolic activity, i.e., “cell index”) may be monitored or measured at predetermined frequencies (e.g., once every 15 minutes) over the course of multiple days. In some cases, live cell proliferation, morphology, and/or viability may be measured by xCELLigence Real Time Cell Analysis Instruments.

Referring to FIG. 2 (left), for the primary T cells from RG1207, the cell index kinetics of the ovarian tumor cells may exhibit a faster cytotoxicity of the target tumor cells by the T cells comprising the system 130, in comparison to that comprising the system 110 or 120, as well as to the control cells. For example, at 40 hours post co-culture, the cell index of the ovarian tumor cells co-cultured with the T cells expressing the system 130 may be about 50% lower than that of the ovarian tumor cells co-cultured with the T cells expressing the system 110 or 120.

Referring to FIG. 2 (right), for the primary T cells from RG1175, the cell index kinetics of the ovarian tumor cells may exhibit a faster cytotoxicity of the target tumor cells by the T cells comprising the system 130, in comparison to that comprising the system 110 or 120, as well as to the control cells. For example, at 40 hours post co-culture, the cell index of the ovarian tumor cells co-cultured with the T cells expressing the system 130 may be about 60% lower than that of the ovarian tumor cells co-cultured with the T cells expressing the system 110 or 120.

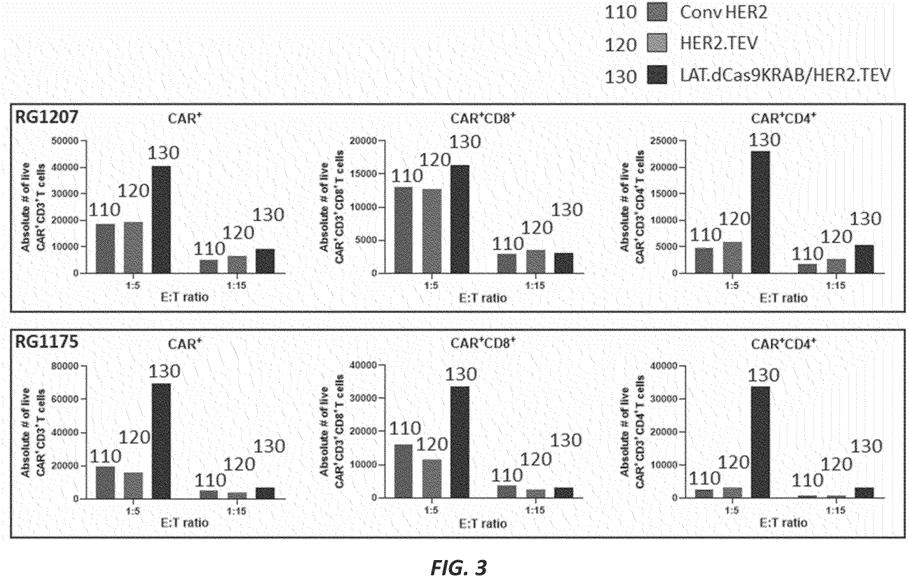

FIG. 3 schematically illustrates effect on T cells’ viability and/or recovery by the three systems 110, 120, and 130, as provided in FIG. 1A. The host primary human T cells may be provided and engineered, as provided and illustrated in FIG. 1B. The effector cells (e.g., T cells transduced with one of the three systems 110, 120, and 130, or control cells) may be cultured with the target cells (e.g., ovarian tumor cells, SKOV3, that carry a luciferase reporter, i.e., “SKOV3.luc”) at an effector-to-target (E:T) ratio (e.g., 1:5 or 1:15), and cultured over the course of multiple days (e.g., 5 days), as provided and illustrated in FIG. 2. After the co-culture with the target cells, the T cells may be harvested, and the expression of the CAR, CD8, and/or CD4 of the T cells may be analyzed by flow cytometry. Data from the flow cytometry may be used to determine an absolute number of live T cells expressing the CAR, CD8, and/or CD4.

Referring to FIG. 3, the presence of the at least one heterologous NES in the adaptor protein of the system 130 may enhance cell viability and/or recovery of the host cell, in comparison to the system 110 and/or the system 120. In some cases, with CAR expression as a cell marker, the system 130 may enhance cell viability and/or recovery of the primary human T cell (e.g. from RG1207 and E:T ratio of 1:5) by at least about 100%, in comparison to the systems 110 or 120. With CAR and CD8 as cell markers, the system 130 may enhance cell viability and/or recovery of the primary human T cell (e.g. from RG1207 and E:T ratio of 1:5) by at least about 30%, in comparison to the systems 110 or 120. With CAR and CD4 as cell markers, the system 130 may enhance cell viability and/or recovery of the primary human T cell (e.g. from RG1207 and E:T ratio of 1:5) by at least about 200%, in comparison to the systems 110 or 120. In some cases, with CAR expression as a cell marker, the system 130 may enhance cell viability and/or recovery of the primary human T cell (e.g. from RG1175 and E:T ratio of 1:5) by at least about 250%, in comparison to the systems 110 or 120. With CAR and CD8 as cell markers, the system 130 may enhance cell viability and/or recovery of the primary human T cell (e.g. from RG1175 and E:T ratio of 1:5) by at least about 120%, in comparison to the systems 110 or 120. With CAR and CD4 as cell markers, the system 130 may enhance cell viability and/or recovery of the primary human T cell (e.g. from RG1175 and E:T ratio of 1:5) by at least about 1,000% (i.e. 10-fold), in comparison to the systems 110 or 120.

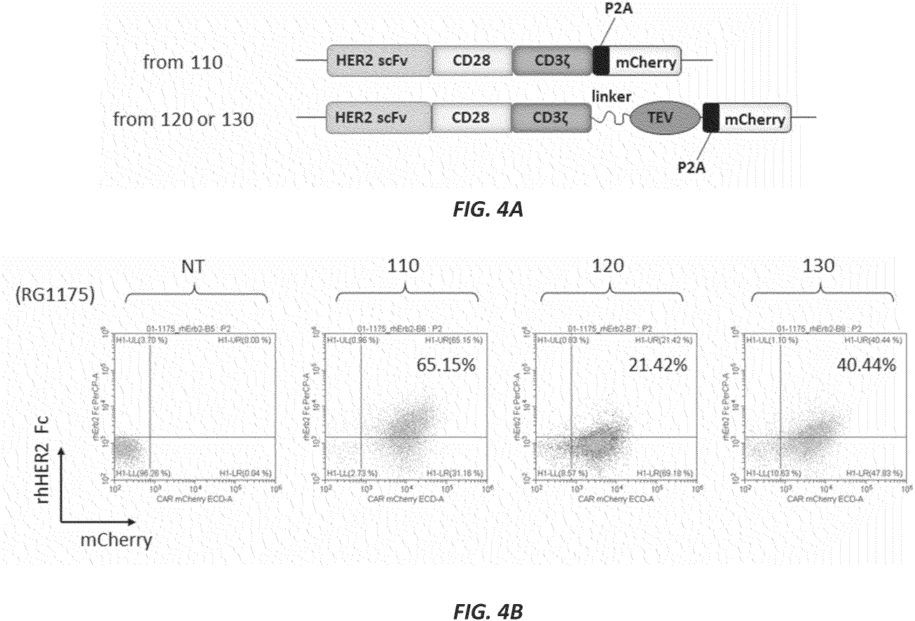

FIG. 4A schematically illustrates portions of two vectors, each respectively encoding a receptor. FIG. 4A (above) may be a portion of a vector encoding the Conv.HER2 CAR of the system 110 (as shown in FIG. 1A), and FIG. 4A (below) may be a portion of a vector encoding the HER2.TEV CAR of the systems 120 and 130 (as shown in FIG. 1A). For both vectors, an end (e.g., the C-terminus) of the receptor may be linked to a reporter protein (e.g., mCherry) via a self-cleavage polypeptide (e.g., porcine teschovirus-1 2A (P2A)).

FIG. 4B schematically illustrates detection of HER2CAR expression of different host T cells by antibody staining. Non-transduced (NT) T cells or T cells transduced with the system 110, 120, or 130, as provided and illustrated in FIG. 2, may be stained with recombinant human HER2-Fc chimera (rhHER2 Fc) and PerCP-conjugated anti-Fc for dual-detection of the HER2CAR in each system. As shown by comparison of the host cells expressing the systems 110 or 120, the addition of the cleavage moiety (e.g., TEV) to the HER2CAR may reduce the proportion of the cells expressing the HER2CAR from about 65% to about 21%. However, as shown by comparison of the host cells expressing the systems 120 or 130, the addition of the at least one heterologous NES to the adaptor protein of the receptor may enhance the proportion of the cells expressing the HER2CAR from about 21% to about 40%. As such, the presence of the at least one heterologous NES linked to the adaptor protein of the receptor may enhance expression and/or stability of the receptor (e.g., the HER2CAR) in the host cells.

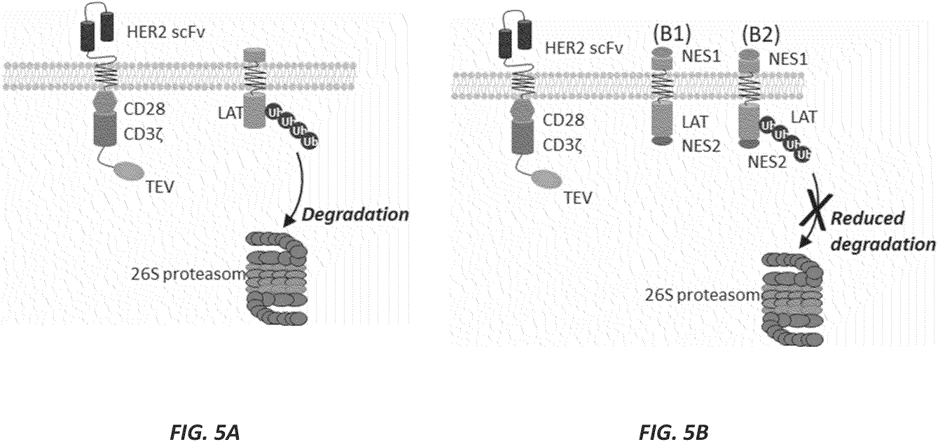

FIGS. 5A and 5B schematically illustrate the effect on ubiquitination and/or proteasome-mediated degradation of an adaptor protein of a receptor in a cell by the absence (FIG. 5A) or presence (FIG. 5B) of at least one heterologous nuclear export signal (NES) linked to the adaptor protein.

Referring to FIG. 5A, an endogenous, non-engineered adaptor protein (e.g., LAT) may undergo ubiquitination, followed by proteasome-mediated degradation (e.g., via 26S proteasome) during and/or in the absence of signaling of a respective receptor. In contrast, referring to FIG. 5B, the cell may comprise the system 130 (as provided in FIG. 5A). In the presence of the at least one heterologous NES linked to the adaptor protein (e.g., the LAT that is flanked by a first heterologous NES and a second heterologous NES), the modified adaptor protein (i) may not undergo any ubiquitination, indicated as “B1” or (ii) may undergo ubiquitination, but may not interact with the proteasome due to reduced chance of displacement from the cell membrane and/or reduced binding to the proteasome, indicated as “B2”.

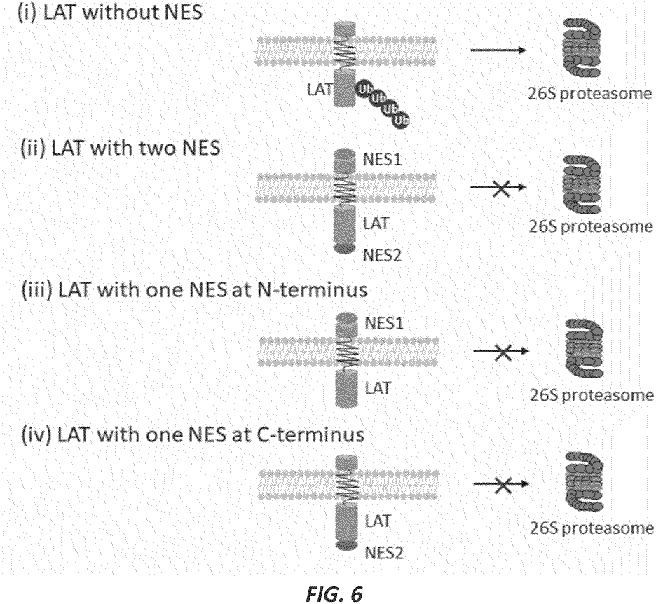

FIG. 6 schematically illustrates various modifications of an adaptor protein (e.g., LAT) of a receptor (e.g., TCR or CAR), and the effect of such modifications on ubiquitination and/or proteasome-mediated degradation of the adaptor protein. As shown in (i), non-engineered LAT may undergo ubiquitination, followed by proteasome-mediated degradation (e.g., via 26S proteasome) during and/or in the absence of signaling of a respective receptor. In contrast, as shown in (ii) through (iv), the LAT linked to at least one or two heterologous NES domains may not undergo any ubiquitination or may undergo reduced ubiquitination as compared to the LAT without any heterologous NES. As a result, the modified LAT may reduce or prevent proteasome-mediated degradation, and improve half-life and/or stability of the LAT in the cell membrane, in comparison to the LAT without any heterologous NES.

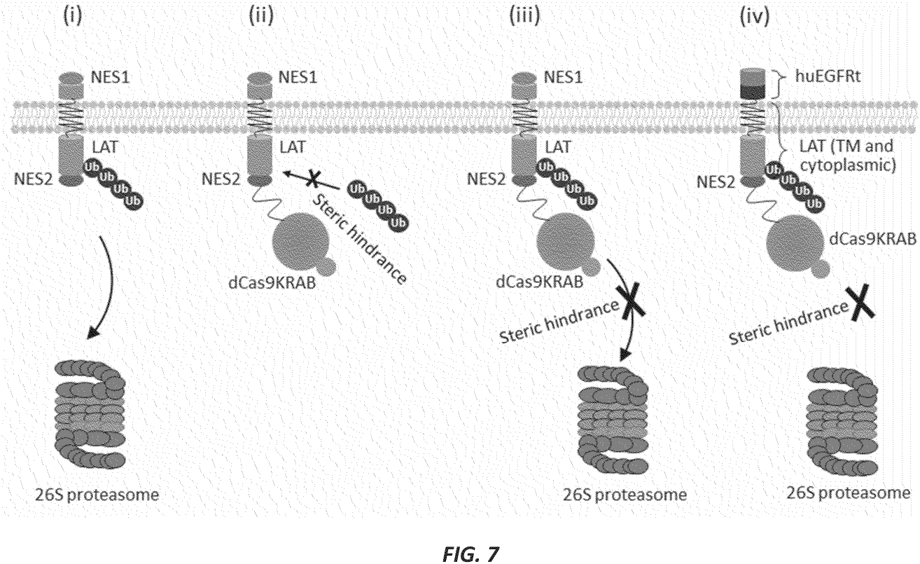

FIG. 7 schematically illustrates another modification of an adaptor protein (e.g., LAT) of a receptor (e.g., TCR or CAR), and the effect of such modification on ubiquitination and/or proteasome-mediated degradation of the adaptor protein. In examples (i) through (iii), the adaptor protein may be the LAT, which may be part of a chimeric polypeptide comprising NES1-LATb-NES2-TCS-dCas9-KRAB, as illustrated in FIG. 1A. Referring to FIG. 7, as shown in (i), non-engineered LAT may undergo ubiquitination, followed by proteasome-mediated degradation (e.g., via 26S proteasome) during and/or in the absence of signaling of a respective receptor. In contrast, as shown in (ii), the presence of an additional polypeptide (e.g., dCas9KRAB) linked to the chimeric polypeptide comprising the LAT and the at least one heterologous NES may block access (e.g., via steric hindrance) of the ubiquitin ligase to its substrate on the LAT, thereby inhibiting ubiquitination of the LAT and thus proteasome-mediated degradation of the LAT. Alternatively, as shown in (iii), the adaptor protein linked to the at least one heterologous NES may still undergo ubiquitination. However, the presence of an additional polypeptide (e.g., dCas9KRAB) linked to the chimeric polypeptide comprising the LAT and the at least one heterologous NES may block access (e.g., via steric hindrance) of one or more proteasomes to the ubiquitin(s), thereby inhibiting or reducing proteasome-mediated degradation of the LAT. In another example, as shown in (iv), the adaptor protein linked to a heterologous NES (i.e., NES2 in the intracellular domain) and a suicide switch (e.g., huEGFRt in the extracellular domain) may still undergo ubiquitination. However, the presence of an additional polypeptide (e.g., dCas9KRAB) linked to the chimeric polypeptide comprising the LAT and the heterologous NES may block access (e.g., via steric hindrance) of one or more proteasomes to the ubiquitin(s), thereby inhibiting or reducing proteasome-mediated degradation of the LAT.

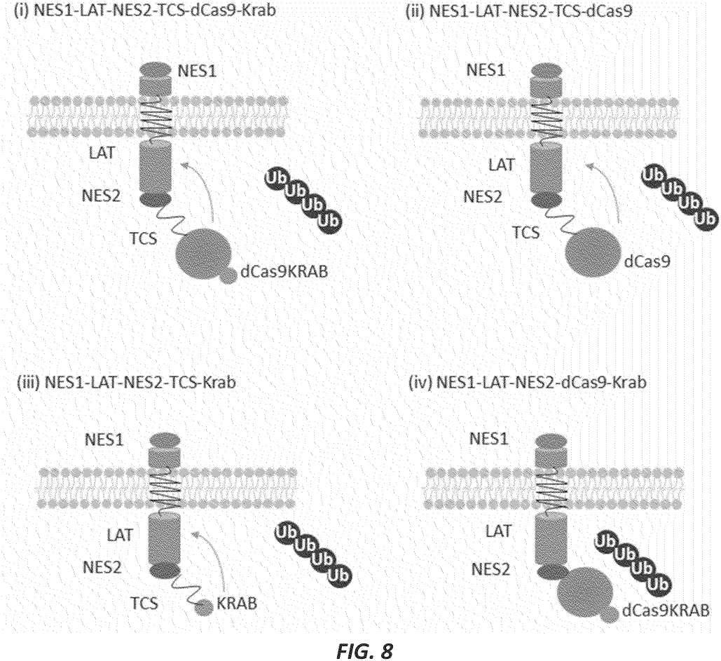

FIG. 8 schematically illustrates various modifications of an adaptor protein (e.g., LAT) of a receptor that may reduce or prevent ubiquitination of the adaptor protein. The presence of an additional polypeptide (e.g., dCas9 and/or KRAB) linked to the chimeric polypeptide comprising the LAT and the at least one heterologous NES may block access (e.g., via steric hindrance) of the ubiquitin ligase to its substrate on the LAT, thereby inhibiting ubiquitination of the LAT and thus proteasome-mediated degradation of the LAT. As shown in (i), the chimeric polypeptide may be NES1-LAT-NES2-TCS-dCas9-Krab, wherein the TCS linker may allow dCas9-KRAB to fold back onto LAT and block access of the ubiquitin ligase to its substrate on the LAT. As shown in (ii), the chimeric polypeptide may be NES1-LAT-NES2-TCS-dCas9, wherein the TCS linker may allow dCas9 to fold back onto LAT and block access of the ubiquitin ligase to its substrate on the LAT. As shown in (iii), the chimeric polypeptide may be NES1-LAT-NES2-TCS-Krab, wherein the TCS linker may allow KRAB to fold back onto LAT and block access of the ubiquitin ligase to its substrate on the LAT. As shown in (iv), the chimeric polypeptide may be NES-1LAT-NEs2-dCas9-Krab, wherein the bulkiness of the dCas9 alone or with Krab may be sufficient to block access of the ubiquitin ligase to its substrate on the LAT. In all of the cases shown in (i) through (iv), reducing or blocking access of the ubiquitination ligase to the LAT may reduce or prevent ubiquitination of the LAT, thereby (1) reducing proteasome-mediated degradation of the LAT and/or (2) enhanced or prolonged signaling of the respective receptor of the adaptor protein.

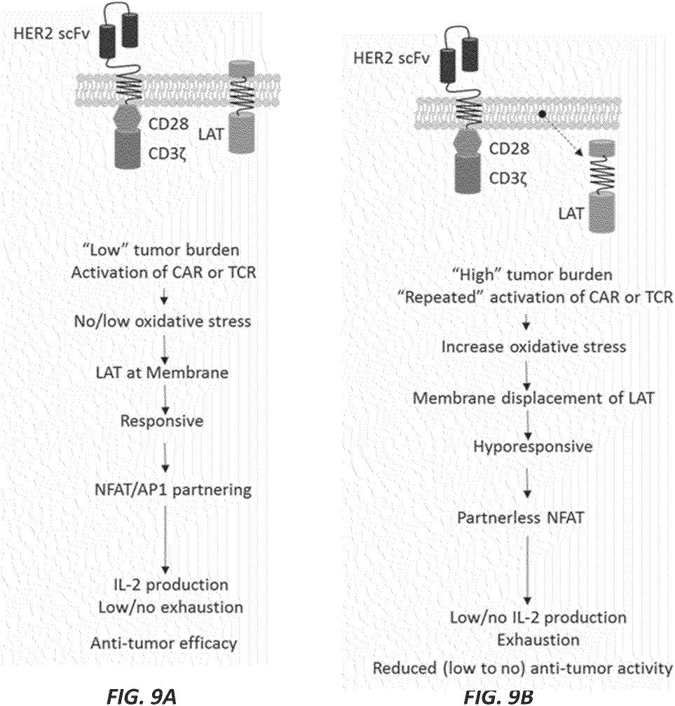

FIG. 9 schematically illustrates effect on anti-tumor activity of T cell by the presence of modified adaptor proteins. As shown in FIG. 9A, the host cell (e.g., T cell) expresses the system 110 comprising a non-modified adaptor protein (e.g., LAT, as shown in FIG. 1A). Under low tumor burden or in the absence of target tumor cells, activation of CAR and its adaptor protein, LAT, in the T cell may be minimal (or at a steady state) and may lead to low oxidative stress. In contrast, as shown in FIG. 9B, under high tumor burden or in the absence of target tumor cells, prolonged or repeated activation of the CAR and its adaptor protein, LAT, may lead to accumulation of high oxidative stress. Such high oxidative stress may cause displacement of the LAT from the cell membrane, resulting in rendering the CAR activity hyporesponsive to the target tumor cells and eventual exhaustion of the LAT.

As shown in FIG. 9C, the host cell (e.g., T cell) expresses the system 130 comprising a modified adaptor protein. The modified adaptor protein may be linked to the at least one heterologous NES (as shown in FIG. 1A) and/or may comprise a mutation of one or more redox-sensitive amino acid residues to render them redox-insensitive. Under high tumor burden or in the absence of target tumor cells, the presence of the at least one heterologous NES and/or the modification of the one or more redox-sensitive amino acid residues may maintain LAT at the cell membrane, thereby enhancing immune synapse, as well as T cell responsiveness and activation without exhaustion. For LAT, examples of the redox-sensitive amino acid sequences may be selected from the group consisting of the following cysteine residues: C9, C26, C29, and C117.

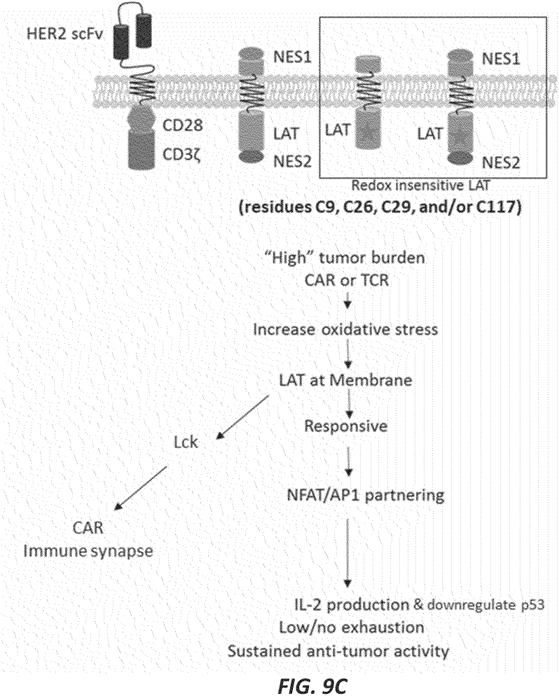

FIG. 10A schematically illustrates a portion of a vector encoding a receptor (e.g., a chimeric receptor) and a modified adaptor protein of the receptor (e.g., human truncated EGFR huEGFRt/LAT), wherein the receptor and the modified adaptor protein are linked by a self-cleavage polypeptide. The receptor may be a CAR comprising anti-HER2 scFv, at least a portion of CD28, and at least a portion of CD3zeta. The modified adaptor protein of the receptor may be at least a portion of a LAT or a functional modification of the LAT, linked to at least a truncated portion of a epidermal growth factor receptor (huEGFRt). The LAT may be linked to at least one heterologous NES to enhance receptor signaling and enhanced anti-tumor activity of the host cell (e.g., T cell). The huEGFRt may serve as a safety switch to deactivate or induce death of the host cell, e.g., by using antibody against the huEGFRt, wherein the antibody comprises a cytotoxic compound.

FIG. 10B schematically illustrates expression of the chimeric receptor and the modified adaptor proteins provided in FIG. 10A. The CAR is shown in (i). As shown in (ii) and (iii), the huEGFRt is fused to the cytoplasmic domain of LAT (ii) or redox-insensitive LAT (iii). The redox-insensitive LAT may be that provided and illustrated in FIG. 9C. The huEGFRt may include a transmembrane domain and EGFR extracellular domain, which can be a target site for Cetuximab. T cells engineered with a construct expressing the CAR may (1) respond to CAR-specific tumor antigen, leading to CD28 and CD3zeta signaling, after which (2) the huEGFRt /LAT may amplify and prolong the CAR activation. To mitigate potential risk of overactive CAR signaling and the resulting adverse events, the huEGFRt may serve as a suicide switch. At the onset of cytokine release syndrome, Cetuximab can be administered to bind extracellular domain of huEGFRt, leading to ADCC-mediated ablation of CAR T cells.

### Example 2: Example LAT Sequences

Human LAT isoform a:

1 meeailvpcv lgllllpila mlmalcvhch rlpgsydsts sdslyprgiq fkrphtvapw
61 ppayppvtsy pplsqpdllp iprspqplgg shrtpssrrd sdgansvasy enegasgirg
121 aqagwgvwgp swtrltpvsl ppepacedad ededdyhnpg ylvvlpdstp atstaapsap
181 alstpgirds afsmesiddy vnvpesgesa easldgsrey vnvsqelhpg aaktepaals
241 sqeaeeveee gapdyenlqe ln (SEQ ID NO: 4)

Human LAT isoform b:

1 meeailvpcv lgllllpila mlmalcvhch rlpgsydsts sdslyprgiq fkrphtvapw
61 ppayppvtsy pplsqpdllp iprspqplgg shrtpssrrd sdgansvasy eneepaceda
121 dededdyhnp gylvvlpdst patstaapsa palstpgird safsmesidd yvnvpesges
181 aeasldgsre yvnvsqelhp gaaktepaal ssqeaeevee egapdyenlq eln (SEQ ID NO: 5)

Human LAT isoform c:

1 meeailvpcv lgllllpila mlmalcvhch rlpgsydsts sdslyprgiq fkrphtvapw
61 ppayppvtsy pplsqpdllp ipspqplggs hrtpssrrds dgansvasye neepacedad
121 ededdyhnpg ylvvlpdstp atstaapsap alstpgirds afsmesiddy vnvpesgesa
181 easldgsrey vnvsqelhpg aaktepaals sqeaeeveee gapdyenlqe ln (SEQ ID NO: 6)

Human LAT isoform d:

1 meataaswqv avpvlggasr plgprgaasl lraplqmeea ilvpcvlgll llpilamlma
61 lcvhchrlpg sydstssdsl yprgiqfkrp htvapwppay ppvtsyppls qpdllpiprs
121 pqplggshrt pssrrdsdga nsvasyenee pacedadede ddyhnpgylv vlpdstpats
181 taapsapals tpgirdsafs mesiddyvnv pesgesaeas ldgsreyvnv sqelhpgaak
241 tepaalssqe aeeveeegap dyenlqeln (SEQ ID NO: 7)

### Example 3: N Terminal NES Sequence

Nucleotide: CTGGCCCTGAAGCTGGCCGGCCTGGACATC (SEQ ID NO: 8)

Amino acid: LALKLAGLDI (SEQ ID NO: 2)

### Example 4: C Terminal NES Sequence

Nucleotide: CTGCAGCTGCCTCCACTGGAGAGACTGACCCTG (SEQ ID NO: 9)

Amino acid: LQLPPLERLTL (SEQ ID NO:3)

### Example 5: Example Chimeric Polypeptide Comprising LAT With Two NES Sequences (the Two NES Sequences are Underlined)

Nucleotide:

ATGGGCCTGGCCCTGAAGCTGGCCGGCCTGGACATC gaggaggccatcct
ggtcccctgcgtgctggggctcctgctgctgcccatcctggccatgttga
tggcactgtgtgtgcactgccacagactgccaggctcctacgacagcaca
tcctcagatagtttgtatccaaggggcatccagttcaaacggcctcacac
ggttgccccctggccacctgcctacccacctgtcacctcctacccacccc
tgagccagccagacctgctccccatcccaagatccccgcagccccttggg
ggctcccaccggacgccatcttcccggcgggattctgatggtgccaacag
tgtggcgagctacgagaacgaggaaccagcctgtgaggatgcggatgagg
atgaggacgactatcacaacccaggctacctggtggtgcttcctgacagc
accccggccactagcactgctgccccatcagctcctgcactcagcacccc
tggcatccgagacagtgccttctccatggagtccattgatgattacgtga
acgttccggagagcggggagagcgcagaagcgtctctggatggcagccgg
gagtatgtgaatgtgtcccaggaactgcatcctggagcggctaagactga
gcctgccgccctgagttcccaggaggcagaggaagtggaggaagaggggg
ctccagattacgagaatctgcaggagctgaacAGCGGCCGCCTGCAGCTG CCTCCACTGGAGAGACTGACCCTG (SEQID NO:10)

Amino acid:

MGLALKLAGLD IEEAILVPCVLGLLLLPILAMLMALCVHCHRLPGSYDST
SSDSLYPRGIQFKRPHTVAPWPPAYPPVTSYPPLSQPDLLPIPRSPQPLG
GSHRTPSSRRDSDGANSVASYENEEPACEDADEDEDDYHNPGYLVVLPDS
TPATSTAAPSAPALSTPGIRDSAFSMESIDDYVNVPESGESAEASLDGSR
EYVNVSQELHPGAAKTEPAALSSQEAEEVEEEGAPDYENLQELNSGRLQL PPLERLTL (SEQ ID NO:1)

### Example 6: Example Ubiquitination Residue(s) of LAT Isoform B

K52 and/or K204

### Example 7: Example Cysteine Residue(s) of LAT Isoform B

C9, C26, C29 and/or C117

### Example 8: System and Methods for Regulating Signaling of a Receptor in a Cell

Any subject system disclosed herein (e.g., a system comprising a chimeric receptor and an adaptor protein of the receptor that comprises one or more heterologous NES sequences) may be tested in vivo (e.g., in a mouse tumor model) to assess the efficacy of the one or more heterologous NES sequences in the adaptor protein. In some examples, the systems 110 and 130 as illustrated in FIG. 1A may be expressed in immune cells (e.g., T cells), and the engineered immune cells may be tested in vivo in a mouse tumor model to study their anti-tumor efficacy. The system 110 may be referred to as “Conv CAR” in this Example. The system 130 without a gRNA (e.g., anti-PD-1 sgRNA) may be referred to as “NOsg” in this Example. The system 130 with the anti-PD-1 sgRNA may be referred to as “PD1sg” in this Example. A plurality of experiment cohorts may be tested in the in vivo study, as shown in Table 4.

TABLE 4

| Cohort # | Treatment | # of cells administered | # of mice | Remarks |
|---|---|---|---|---|
| 1 | PBS (Background control) | - | 7 | - |
| 2 | Non-engineered T cells | 3 million | 7 | - |
| 3 | Non-engineered T cells | 3 million | 7 | with Atezolizumab |
| 4 | Conv CAR T cells | 3 million | 7 | - |
| 5 | Conv CAR T cells | 3 million | 7 | with Atezolizumab |
| 6 | Conv CAR T cells | 1 million | 7 | - |
| 7 | Conv CAR T cells | 1 million | 7 | with Atezolizumab |
| 8 | NOsg T cells | 3 million | 7 | - |
| 9 | NOsg T cells | 3 million | 7 | with Atezolizumab |
| 10 | NOsg T cells | 1 million | 7 | - |
| 11 | NOsg T cells | 1 million | 7 | with Atezolizumab |
| 12 | NOsg T cells | 1 million | 7 | with Atezolizumab, until Day 27 |
| 13 | PD1sg T cells | 3 million | 8 | - |
| 13 | PD1sg T cells | 1 million | 8 | - |

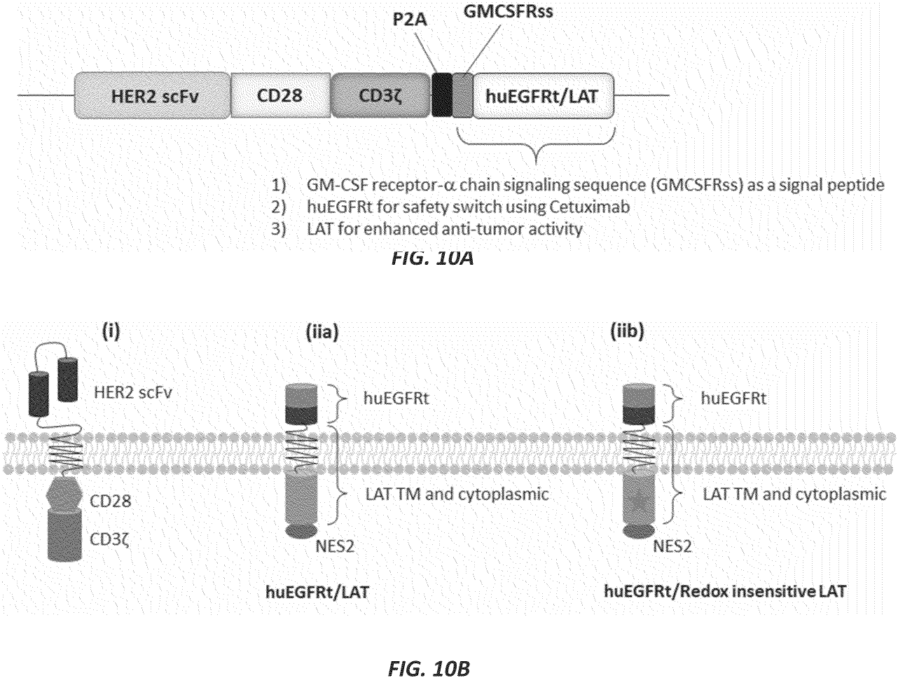

As show in FIG. 11, T cells transduced with HER2-TEV receptor and LAT-dCas9-KRAB adaptor protein (LAT-dCas9-KRAB/HER2 CAR-TEV) had better proliferation and cytokine production than T cells transduced with conventional HER2 CAR during killing of tumor cells. Head and neck tumor cells, FaDu carrying luciferase reporter and overexpression of PD-L1, were seeded in 48-well plates. 24 hours later, non-transduced (NT) T cells, conventional HER2-CAR-transduced T cells, LAT-dCas9-KRAB/HER2 CAR-TEV-transduced T cells (NOsg), or LAT-dCas9-KRAB/HER2 CAR-TEV/PD1sg-transduced T cells (PD1sg) were co-cultured at 1:20 effector to target ratio for 6 days. Kinetics of live tumor cells proliferated CAR+ T cells were monitored at day 1, 2, 3, and 6 by flow cytometry analysis. Supernatant of the co-culture was analyzed by ELISA for the detections of secreted IL2 and TNFα. T cells transduced with LAT-dCas9-KRAB/HER2 CAR-TEV/PD1sg had shown an increased number of proliferation starting on day 3 (bottom left panel of FIG. 11) compared to T cells that were either transduced with conventional HER2-CAR only (Conv CAR) or with LAT-dCas9-KRAB/HER2 CAR-TEV (NOsg). T cells transduced with LAT-dCas9-KRAB/HER2 CAR-TEV/PD1sg had also shown increased secretions of IL2 (top right panel of FIG. 11) and TNFα (bottom right panel of FIG. 11) starting on day 2 of the assay when compared to IL2 and TNFα secreted by T cells that were either transduced with conventional HER2-CAR only (Conv CAR) or with LAT-dCas9-KRAB/HER2 CAR-TEV (NOsg).

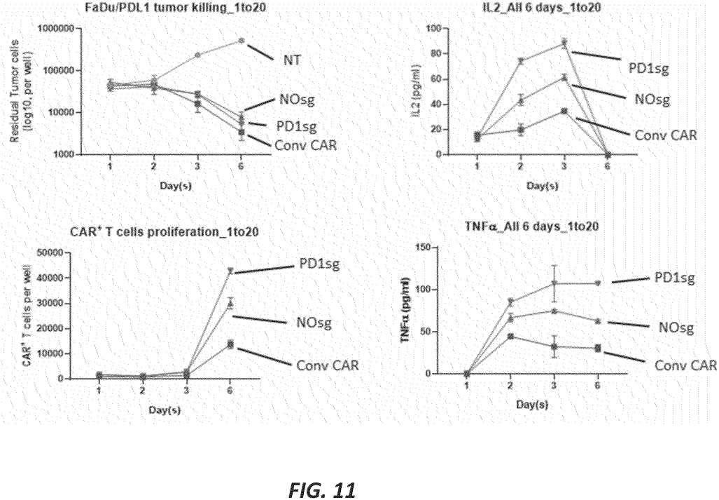

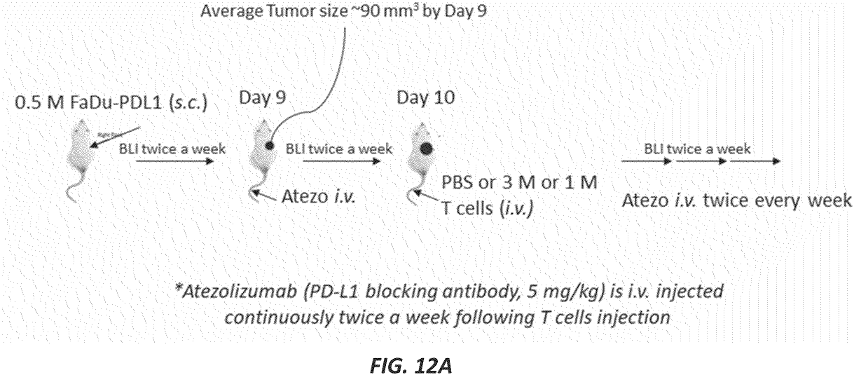

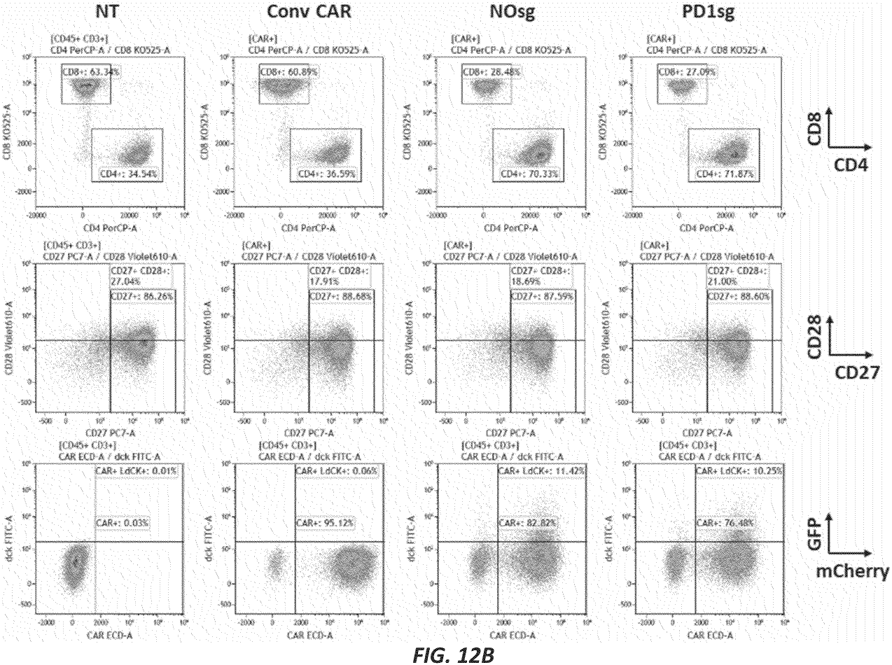

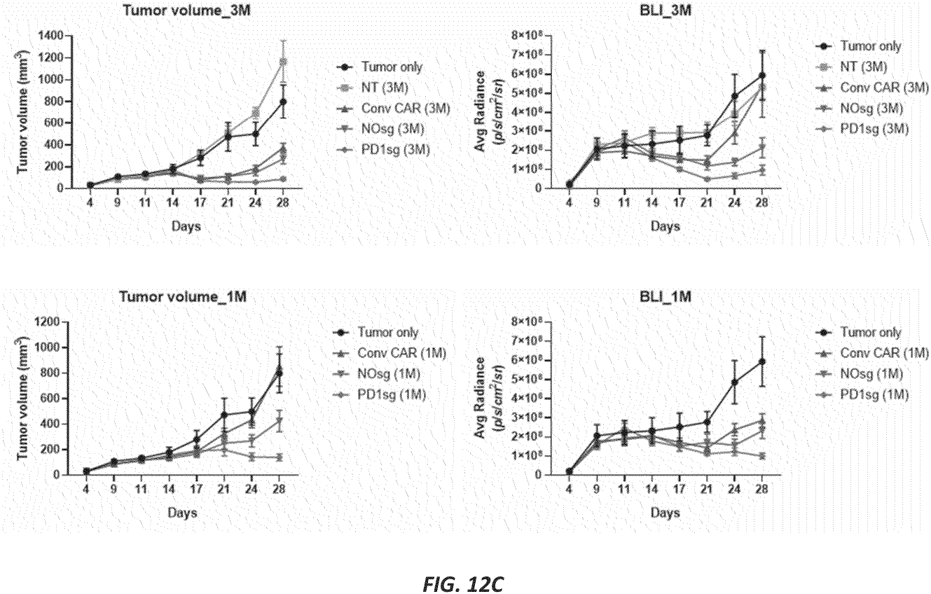

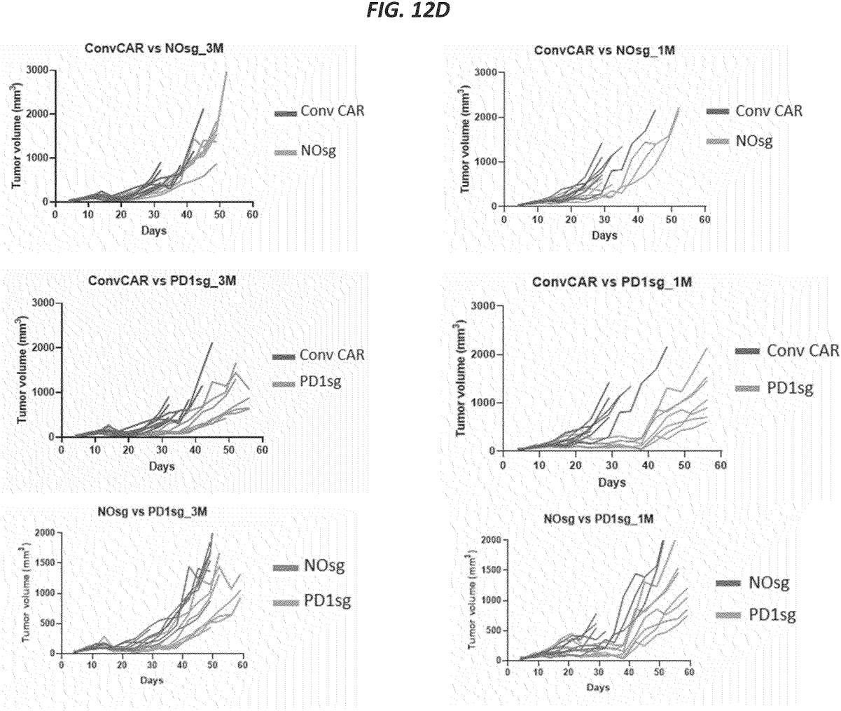

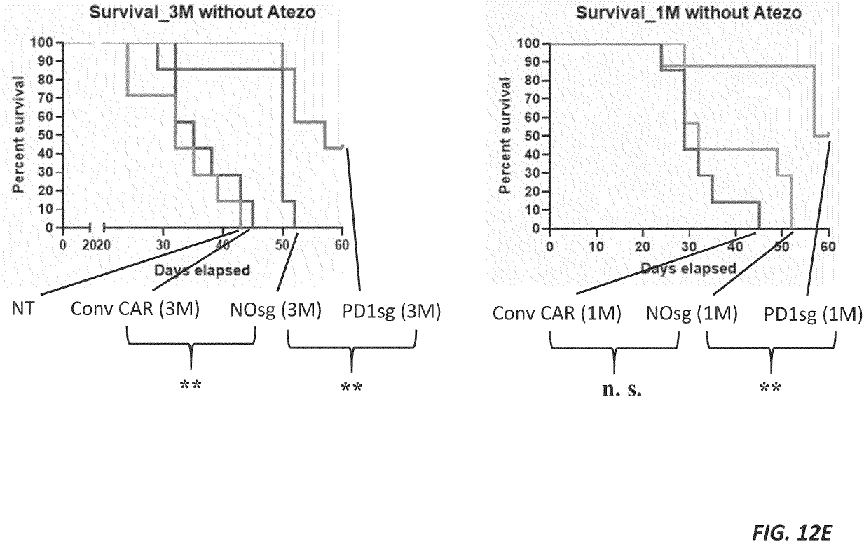

FIG. 12A illustrates experiments for examining the in vivo tumor cell killing activity of the T cells transduced with the systems comprising a receptor (e.g., a chimeric receptor) and an adaptor protein of the receptor as described herein. 0.5 million (0.5 M) PD-L1 positive squamous cell carcinoma cells (FaDu-PLD1 cells) were transplanted into NSG mice via subcutaneous (s.c.) injection. 1 × 10⁶ (1 M) or 3 × 10⁶ (3 M) of T cells that were not transduced (NT), transduced with conventional CAR (Conv CAR), transduced with LAT-dCas9-KRAB/HER2 CAR-TEV (NOsg system), or LAT-dCas9-KRAB/HER2 CAR-TEV/PD1sg (PD1sg system) were systemically administered 10 days after transplantation of the FaDu tumor cells. The bioluminescence of FaDu-PDL1 cells and size of tumor was monitored at different time points by bioluminescence imaging (BLI). As shown in FIG. 12B, the remaining T cells not used for injection were examined by phenotyping of surface expression markers prior to these T cells being activated. T cells transduced with the NOsg system or the PD1sg system exhibited a higher proportion of T cells that are CD4⁺/CD8⁺ in comparison to T cells transduced with the Conv CAR system or control T cells (top row of FIG. 12B). In some cases, CD4⁺ T cells can be referred to as helper T cells, while CD8⁺ T cells can be referred to as cytotoxic T cells. Both CD8⁺ and CD4⁺ T cells can kill tumor cells, but the CD4⁺ T cells can persist cell killing activity longer. In some cases, without wishing to be bound by theory, a presence of LAT linked to one or more heterologous NES can induce the T cell to differentiate into CD4⁺ T cells. In some cases, without wishing to be bound by theory, CD4⁺ T cells can persist longer in the body than other types of T cells (e.g., CD8⁺ killer T cells), and CD4⁺ T cells can also target and kill tumor/cancer cells.

Also shown in FIG. 12B, the T cells transduced with LAT-dCas9-KRAB/HER2 CAR-TEV/PD1sg (PD1sg) exhibited a comparable proportion of cells positive for CD27, a marker for T cell activation and differentiation, relative to other T cell groups, e.g., the NOsg system and the Conv CAR system (middle row of FIG. 12B). T cells belonging to the PD1sg system, the NOsg system, and the Conv CAR can be proliferative, differentiating, and active for targeting and/or killing tumor cells.

The last/bottom row of FIG. 12B illustrates that T cells belonging to the PD1sg group had expressed comparable levels of CAR, in comparison to T cells belonging to groups of Conv CAR and NOsg as indicated by the proportion of T cells positive for mCherry-tagged CAR and GFP-tagged LAT. In some cases, NOsg or PD1sg systems can exhibit a lower expression profile of the CAR than Conv CAR.

As shown in FIG. 12C, tumor volume and bioluminescence were measured at indicated time points. Data was presented as means ± SEM (n = 7-8). Top panels of FIG. 12C showed that 3 × 10⁶ (3 M) T cells transduced with the PD1sg system were effective at decreasing tumor volume (top left panel) and BLI measurements (top right panel) in mice compared to untreated mice (Tumor only), mice treated with 3 M non-transduced T cells (NT), mice treated with 3 M T cells transduced with Conv CAR, and mice treated with 3 M T cells transduced with NOsg system. Similar observations of decreasing tumor volumes (bottom left panel) and BLI measurements (bottom right panel) were made in the mice treated with 1 × 10⁶ (1 M) T cells transduced with the PD1sg system.

FIG. 12D illustrates comparisons of tumor volumes measured for each individual mouse at different time points. Top row illustrates that administering 3 × 10⁶ (3 M, top left panel) or 1 × 10⁶ (1 M, top right panel) T cells transduced with NOsg system moderately delayed the growth of tumor volume in comparison to T cells transduced with Conv CAR. Middle and bottom rows illustrate that T cells transduced with the PD1sg were effective at delaying the growth of tumor volumes in comparison to both T cells transduced with Conv CAR (middle row) and T cells transduced with the NOsg system. Middle left panel illustrates that the 3 × 10⁶ (3 M) T cells transduced with the PD1sg system were more effective at delaying the growth of tumor volumes compared to 3 × 10⁶ (3 M) T cells transduced with Conv CAR. Middle right panel illustrates that the 1 × 10⁶ (1 M) T cells transduced with the PD1sg system were more effective at delaying the growth of tumor volumes compared to 1 × 10⁶ (1 M) T cells transduced with Conv CAR. Bottom left panel illustrates that the 3 × 10⁶ (3 M) T cells transduced with the PD1sg system were more effective at delaying the growth of tumor volumes compared to 3 × 10⁶ (3 M) T cells transduced with the NOsg system. Bottom right panel illustrates that the 1 × 10⁶ (1 M) T cells transduced with the PD1sg system were more effective at delaying the growth of tumor volumes compared to 1 × 10⁶ (1 M) T cells transduced with the NOsg system.

FIG. 12E illustrates Kaplan-Meier survival curves for 3 × 10⁶ (3 M) cells or 1 × 10⁶ (1 M) cells dose group of administration of HER2 CAR T cells. The end point was established as a tumor volume of >2,000 mm³. *P < 0.05; **P < 0.01; ***P < 0.001. Mice treated with 3 M (left panel) or 1 M (right panel) of T cells transduced with the PD1sg system had shown significant longer survivals compared to untreated mice (NT), mice treated with T cells transduced with Conv CAR, and mice treated with T cells transduced with NOsg system. FIG. 12E also illustrates that the T cells transduced with NOsg were more effective at extending the lifespan of the mice than the untreated mice and the mice treated with Conv CAR. Without wishing to be bound by theory, a presence of T cells comprising a LAT linked to one or more heterologous NES sequences can enhance survival (or lifespan) of a subject having or suspected of having a tumor/cancer, in comparison to a presence of T cells comprising a naïve or endogenous LAT without any heterologous NES sequence.

Upon reaching the end point of the experiments of FIG. 12E, mice were euthanized and tumors were harvested for flow cytometry analysis as shown in FIG. 12F. Using hCD45 as a marker, the % of lymphocytes in total tumor mass was plotted between groups as indicated in the left panel. The % of lymphocytes was increased in mice treated with both T cells transduced with the PD1sg system and T cells transduced with the NOsg system, in comparison to that in mice treated with T cells transduced with Conv CAR. Expression of PD1 in the lymphocytes in total tumor mass between the groups was also compared, as shown in the right panel of FIG. 12F. Data was presented as means ± SEM (n = 3-8). PD1 expression in the lymphocytes was lower in mice treated with T cells transduced with the PD1sg system than that in mice treated with T cells transduced with either the NOsg system or the Conv CAR system.

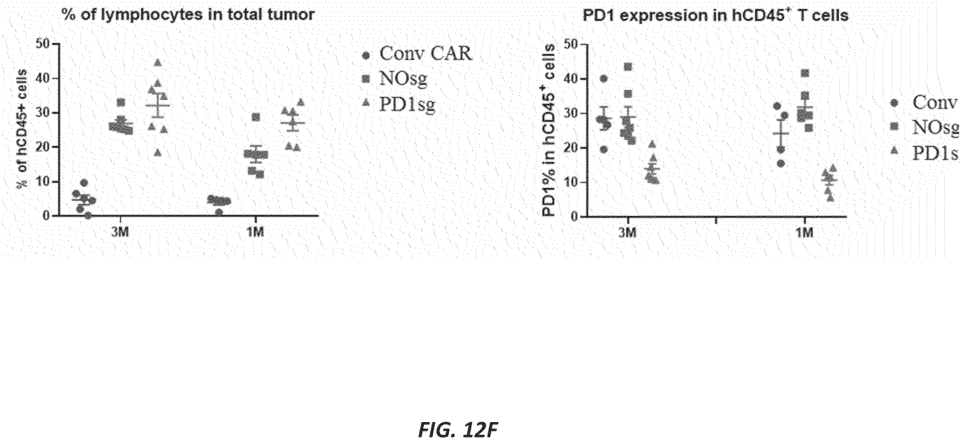

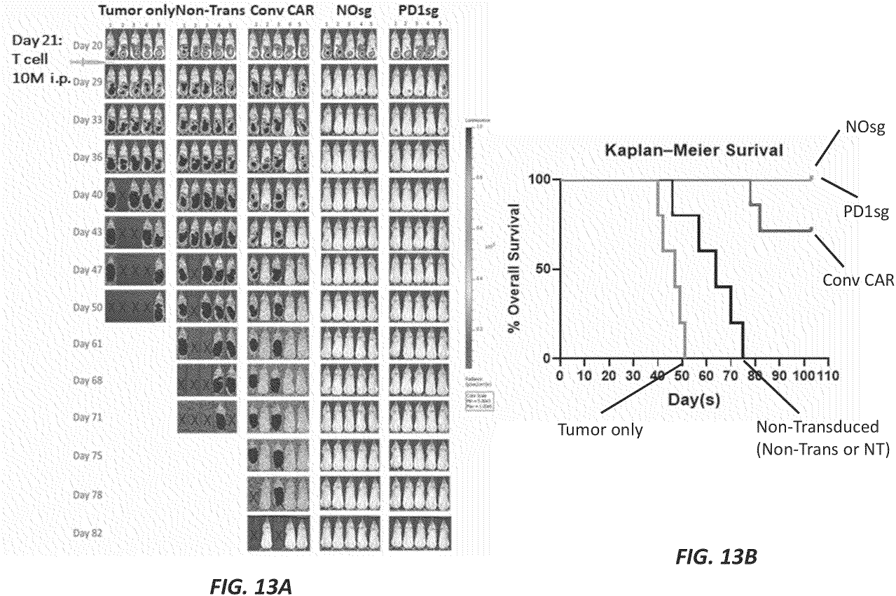

FIG. 13 illustrates how the T cells transduced with the LAT-dCas9-KRAB/HER2CAR-TEV system enhanced the anti-tumor effects of HER2 CAR T cells against SKOV3 tumor cells in vivo. FIG. 13A shows that the SKOV3 tumor cells expressing luciferase (10 million) were injected intraperitoneally into NSG mice. 10 × 10⁶ of non-transduced T cells, T cells transduced with Conv CAR, T cells transduced with the NOsg system, or T cells transduced with PD1sg were intraperitoneally administered 21 days after transplantation of tumor cells in the NSG mice. The bioluminescence of SKOV3 cells was monitored at different time points as indicated. Mice treated with T cells transduced with PD1sg system or the NOsg system had shown longer survival and/or decreased BLI measurements compared to untreated mice (Tumor only), mice treated with non-transduced T cells (Non-Trans), and mice treated with T cells transduced with Conv CAR. FIG. 13B illustrates Kaplan-Meier survival curves after administration of HER2 CAR T cells to the mice of FIG. 13A. Mice treated with T cells transduced with either the NOsg system or the PD1sg system had shown increased survival rate compared to untreated mice (Tumor only), mice treated with NT T cells, and mice treated with T cells transduced with Conv CAR. Without wishing to be bound by theory, even in the absence of the ability to target and downregulate the expression of PD1 in the T cells by action of a functioning actuator-PD1 sgRNA unit (i.e., the NOsg system), the presence of the anti-HER2 CAR and the modified LAT comprising one or more heterologous NES sequences can be sufficient to enhance survival and/or anti-tumor/cancer activity, thereby enhancing survival of the subject with the tumor/cancer.

### Example 9: Modifications of the Systems and Methods for Regulating Signaling of a Receptor in a Cell

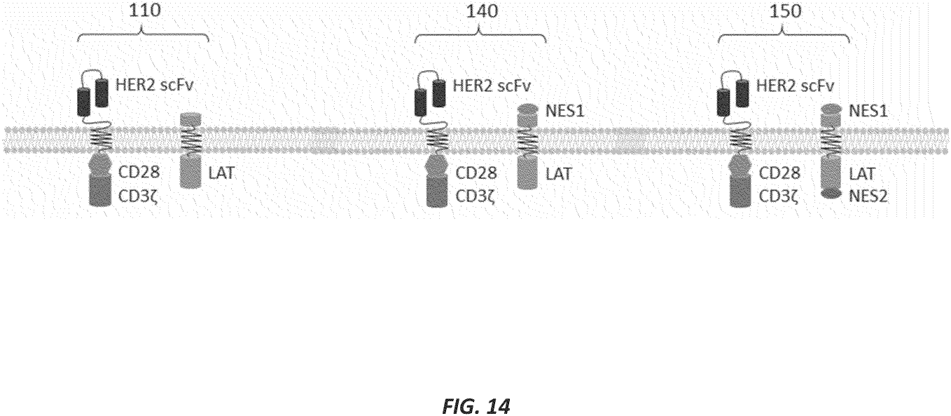

FIG. 14 schematically illustrates two additional systems (140 and 150) based on modifications of system 110. System 110 (i.e., “Conventional HER2” or “Conv.HER2”) comprises a CAR that includes a ligand binding antibody (e.g., anti-HER2 scFv), a transmembrane domain, and an intracellular domain. The intracellular domain of the CAR comprises (i) at least a portion of a signaling domain of CD28 and/or (ii) at least a portion of a signaling domain of CD3zeta. The system 110 further comprises a wild-type adaptor protein of a cell (e.g., an adaptor protein of a T-cell receptor (TCR) of the cell, such as the linker for activation of T cell (LAT)). The wild-type adaptor protein may be endogenous or exogenous to the cell.

System 140 or 150 comprises a receptor (e.g., a chimeric receptor) and an adaptor protein of the receptor. The adaptor protein may be a wild-type adaptor protein. Alternatively, the adaptor protein may be a chimeric adaptor protein comprising one heterologous nuclear localization signal (NES) on the N-terminus of the adaptor protein as shown in system 140 or two heterologous NES flanking the adaptor protein as shown in system 150. In some cases, the adaptor protein as shown in system 140 may be LAT. In some cases, the adaptor protein as shown in system 150 may be LAT.

In some embodiments, cells (e.g., lymphocytes such as T cells) transduced with system 140 or system 150 may be more proliferative compared to cells transduced with system 110. In some embodiments, the proliferation of the cells transduced with system 140 or system 150 may be higher than the proliferation of the cells transduced with system 110 by about 1.1 folds to about 1,000 folds. In some embodiments, the proliferation of the cells transduced with system 140 or system 150 may be higher than the proliferation of the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the proliferation of the cells transduced with system 140 or system 150 may be higher than the proliferation of the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the proliferation of the cells transduced with system 140 or system 150 may be higher than the proliferation of the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the proliferation of the cells transduced with system 140 or system 150 may be higher than the proliferation of the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may be more viable compared to cells transduced with system 110. In some embodiments, the viability of the cells transduced with system 140 or system 150 may be higher than the viability of the cells transduced with system 110 by about 1.1 folds to about 1,000 folds. In some embodiments, the viability of the cells transduced with system 140 or system 150 may be higher than the viability of the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the viability of the cells transduced with system 140 or system 150 may be higher than the viability of the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the viability of the cells transduced with system 140 or system 150 may be higher than the viability of the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the viability of the cells transduced with system 140 or system 150 may be higher than the viability of the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may be more adapt at migrating with or without chemotaxis signaling compared to cells transduced with system 110. In some embodiments, the migration of the cells transduced with system 140 or system 150 may be higher than the migration of the cells transduced with system 110 by about 1.1 folds to about 1,000 folds. In some embodiments, the migration of the cells transduced with system 140 or system 150 may be higher than the migration of the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the migration of the cells transduced with system 140 or system 150 may be higher than the migration of the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the migration of the cells transduced with system 140 or system 150 may be higher than the migration of the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the migration of the cells transduced with system 140 or system 150 may be higher than the migration of the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may exhibit enhanced intracellular signaling (e.g., enhanced TCR-related intracellular signaling) compared to cells transduced with system 110. In some embodiments, the intracellular signaling of the cells transduced with system 140 or system 150 may be higher than the intracellular signaling of the cells transduced with system 110 by about 1.1 folds to about 1,000 folds. In some embodiments, the intracellular signaling of the cells transduced with system 140 or system 150 may be higher than the intracellular signaling of the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the intracellular signaling of the cells transduced with system 140 or system 150 may be higher than the intracellular signaling of the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the intracellular signaling of the cells transduced with system 140 or system 150 may be higher than the intracellular signaling of the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the intracellular signaling of the cells transduced with system 140 or system 150 may be higher than the intracellular signaling of the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may induce more cell cytotoxicity (e.g., against a target cell, such as a tumor/cancer cell) compared to cells transduced with system 110. In some embodiments, the cell cytotoxicity of the cells transduced with system 140 or system 150 may be higher than the cell cytotoxicity of the cells transduced with system 110 by about 1.1 folds to about 1,000 folds. In some embodiments, the cell cytotoxicity of the cells transduced with system 140 or system 150 may be higher than the cell cytotoxicity of the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the cell cytotoxicity of the cells transduced with system 140 or system 150 may be higher than the cell cytotoxicity of the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the cell cytotoxicity of the cells transduced with system 140 or system 150 may be higher than the cell cytotoxicity of the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the cell cytotoxicity of the cells transduced with system 140 or system 150 may be higher than the cell cytotoxicity of the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may secrete more cytokines (e.g., IL-12, IL-23, IL-27, IL-30, IL-35, or any cytokines as disclosed herein) compared to cytokines secreted by the cells transduced with system 110. In some embodiments, the cytokine secretion by the cells transduced with system 140 or system 150 is higher than cytokine secretion by the cells transduced with system 110 by about 1.1 folds to about 1,000 folds. In some embodiments, the cytokine secretion by the cells transduced with system 140 or system 150 is higher than cytokine secretion by the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the cytokine secretion by the cells transduced with system 140 or system 150 is higher than cytokine secretion by the cells transduced with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the cytokine secretion by the cells transduced with system 140 or system 150 is higher than cytokine secretion by the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the cytokine secretion by the cells transduced with system 140 or system 150 is higher than cytokine secretion by the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some embodiments, cells (e.g., lymphocytes such as T cells) transduced with system 140 or system 150 may exhibit enhanced ability to reduce a size of or obliterate a tumor compared to the cells transduced with system 110. In some embodiments, the ability to reduce a size of or obliterate a tumor by the cells transduced with system 140 or system 150 is higher than the ability to reduce a size of or obliterate a tumor against target cells by the cells transduced with system 110 by about 1.1 folds to about 1,000 folds. In some embodiments, the ability to reduce a size of or obliterate a tumor by the cells transduced with system 140 or system 150 is higher than the ability to reduce a size of or obliterate a tumor by the cells transduced with system 110 by about 1.1 folds to about 1.2 folds, about 1.1 folds to about 1.5 folds, about 1.1 folds to about 2 folds, about 1.1 folds to about 2.5 folds, about 1.1 folds to about 5 folds, about 1.1 folds to about 10 folds, about 1.1 folds to about 20 folds, about 1.1 folds to about 50 folds, about 1.1 folds to about 100 folds, about 1.1 folds to about 500 folds, about 1.1 folds to about 1,000 folds, about 1.2 folds to about 1.5 folds, about 1.2 folds to about 2 folds, about 1.2 folds to about 2.5 folds, about 1.2 folds to about 5 folds, about 1.2 folds to about 10 folds, about 1.2 folds to about 20 folds, about 1.2 folds to about 50 folds, about 1.2 folds to about 100 folds, about 1.2 folds to about 500 folds, about 1.2 folds to about 1,000 folds, about 1.5 folds to about 2 folds, about 1.5 folds to about 2.5 folds, about 1.5 folds to about 5 folds, about 1.5 folds to about 10 folds, about 1.5 folds to about 20 folds, about 1.5 folds to about 50 folds, about 1.5 folds to about 100 folds, about 1.5 folds to about 500 folds, about 1.5 folds to about 1,000 folds, about 2 folds to about 2.5 folds, about 2 folds to about 5 folds, about 2 folds to about 10 folds, about 2 folds to about 20 folds, about 2 folds to about 50 folds, about 2 folds to about 100 folds, about 2 folds to about 500 folds, about 2 folds to about 1,000 folds, about 2.5 folds to about 5 folds, about 2.5 folds to about 10 folds, about 2.5 folds to about 20 folds, about 2.5 folds to about 50 folds, about 2.5 folds to about 100 folds, about 2.5 folds to about 500 folds, about 2.5 folds to about 1,000 folds, about 5 folds to about 10 folds, about 5 folds to about 20 folds, about 5 folds to about 50 folds, about 5 folds to about 100 folds, about 5 folds to about 500 folds, about 5 folds to about 1,000 folds, about 10 folds to about 20 folds, about 10 folds to about 50 folds, about 10 folds to about 100 folds, about 10 folds to about 500 folds, about 10 folds to about 1,000 folds, about 20 folds to about 50 folds, about 20 folds to about 100 folds, about 20 folds to about 500 folds, about 20 folds to about 1,000 folds, about 50 folds to about 100 folds, about 50 folds to about 500 folds, about 50 folds to about 1,000 folds, about 100 folds to about 500 folds, about 100 folds to about 1,000 folds, or about 500 folds to about 1,000 folds. In some embodiments, the ability to reduce a size of or obliterate a tumor by the cells transduced with system 140 or system 150 is higher than the ability to reduce a size of or obliterate a tumor with system 110 by about 1.1 folds, about 1.2 folds, about 1.5 folds, about 2 folds, about 2.5 folds, about 5 folds, about 10 folds, about 20 folds, about 50 folds, about 100 folds, about 500 folds, or about 1,000 folds. In some embodiments, the ability to reduce a size of or obliterate a tumor by the cells transduced with system 140 or system 150 is higher than the ability to reduce a size of or obliterate a tumor by the cells transduced with system 110 by at least about 1.1 folds, about 1.2 folds, about 1.5 folds, about 2 folds, about 2.5 folds, about 5 folds, about 10 folds, about 20 folds, about 50 folds, about 100 folds, or about 500 folds. In some embodiments, the ability to reduce a size of or obliterate a tumor by the cells transduced with system 140 or system 150 is higher than the ability to reduce a size of or obliterate a tumor by the cells transduced with system 110 by at most about 1.2 folds, about 1.5 folds, about 2 folds, about 2.5 folds, about 5 folds, about 10 folds, about 20 folds, about 50 folds, about 100 folds, about 500 folds, or about 1,000 folds.

In some instances, cells transduced with system 140 and cells transduced with system 150 may have similar proliferation rates. In some instances, cells transduced with system 140 and cells transduced with system 150 may have similar adaptation for migration. In some instances, cells transduced with system 140 and cells transduced with system 150 may induce similar cytotoxicity against a target cell. In some instances, cells transduced with system 140 and cells transduced with system 150 may secrete similar levels of cytokines. In some instances, cells transduced with system 140 and cells transduced with system 150 may have similar degree of intracellular signaling. In some instances, cells transduced with system 140 and cells transduced with system 150 may have similar ability to reduce a size of or obliterate a tumor.

In some instances, proliferation rate may be higher in cells transduced with system 140 compared to cells transduced with system 150. In some instances, cells transduced with system 140 may be more adapt at migration compared to cells transduced with system 150. In some instances, cells transduced with system 140 may induce more cytotoxicity compared to cells transduced with system 150. In some cases, cells transduced with system 140 may secrete more cytokines compared to cells transduced with system 150. In some instances, cells transduced with system 140 may exhibit a higher degree of intracellular signaling compared to cells transduced with system 150. In some instances, cells transduced with system 140 may exhibit an enhanced ability to reduce a size of or obliterate a tumor compared to cells transduced with system 150.

In some embodiments, proliferation rate may be lower in cells transduced with system 140 compared to cells transduced with system 150. In some instances, cells transduced with system 140 may be less adapt at migration compared to cells transduced with system 150. In some instances, cells transduced with system 140 may induce less cytotoxicity compared to cells transduced with system 150. In some cases, cells transduced with system 140 may secrete less cytokines compared to cells transduced with system 150. In some instances, cells transduced with system 140 may exhibit a lower degree of intracellular signaling compared to cells transduced with system 150. In some instances, cells transduced with system 140 may exhibit a reduced ability to reduce a size of or obliterate a tumor compared to cells transduced with system 150.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1-165. (canceled)

166. A chimeric polypeptide comprising a plurality of heterologous nuclear export signal (NES) domains linked to an adaptor protein of a receptor,
wherein the adaptor protein comprises at least a portion of a Linker for Activation of T cells (LAT).

167. The chimeric polypeptide of claim 166, wherein:
(a) the receptor is a chimeric antigen receptor (CAR); or
(b) the receptor is a T cell receptor (TCR).

168. The chimeric polypeptide of claim 166, wherein the plurality of heterologous NES domains comprises a first heterologous NES domain and a second heterologous NES domain, wherein the adaptor protein is disposed between the first heterologous NES domain and the second heterologous NES domain.

169. The chimeric polypeptide of claim 168, wherein the first heterologous NES domain is disposed in an extracellular region of the chimeric polypeptide and the second heterologous NES domain is in an intracellular region of the chimeric polypeptide.

170. The chimeric polypeptide of claim 166, wherein upon introduction of the chimeric polypeptide into a cell comprising the receptor, the chimeric polypeptide prolongs or enhances signaling of the receptor in the cell, as compared to the adaptor protein without the plurality of heterologous NES domains.

171. The chimeric polypeptide of claim 170, wherein the plurality of heterologous NES domains enhances translocation of the adaptor protein into a membrane of the cell.

172. The chimeric polypeptide of claim 170, wherein the plurality of heterologous NES domains reduces displacement of the adaptor protein from a membrane of the cell during the signaling of the receptor.

173. The chimeric polypeptide of claim 170, wherein the plurality of heterologous NES domains reduces degradation of the adaptor protein during the signaling of the receptor.

174. The chimeric polypeptide of claim 170, wherein the plurality of heterologous NES domains stabilizes the adaptor protein in a membrane of the cell, as compared to the adaptor protein without the plurality of heterologous NES domains.

175. The chimeric polypeptide of claim 166, wherein a heterologous NES domain of the plurality of heterologous NES domains comprises a polynucleotide sequencing having the pattern of LxxLxL, wherein each L is a hydrophobic amino acid residue selected from the group consisting of leucine, isoleucine, valine, phenylalanine, and methionine.

176. The chimeric polypeptide of claim 166, wherein a heterologous NES domain of the plurality of heterologous NES domains comprises a polynucleotide sequencing having the pattern of LxxxLxL, wherein each L is a hydrophobic amino acid residue selected from the group consisting of leucine, isoleucine, valine, phenylalanine, and methionine.

177. The chimeric polypeptide of claim 166, wherein a heterologous NES domain of the plurality of heterologous NES domains comprises a polynucleotide sequencing having the pattern of LxxxLxxLxL, wherein each L is a hydrophobic amino acid residue selected from the group consisting of leucine, isoleucine, valine, phenylalanine, and methionine.

178. The chimeric polypeptide of claim 166, wherein the at least the portion of the LAT comprises at least one mutation, as compared to a wild-type LAT.

179. The chimeric polypeptide of claim 178, wherein the at least one mutation is at one or more cysteine residues of the wild-type LAT..

180. The chimeric polypeptide of claim 178, wherein the at least one mutation is at one or more lysine residues of the wild-type LAT.

181. The chimeric polypeptide of claim 166, wherein the chimeric polypeptide further comprises at least one additional polypeptide, wherein upon introduction of the chimeric polypeptide into a cell, a charge, size, and/or position of the at least one additional polypeptide relative to the chimeric polypeptide is sufficient to inhibit or reduce interaction between the adaptor protein and a cellular component of the cell.

182. The chimeric polypeptide of claim 181, wherein:
(a) the at least one additional polypeptide reduces degradation of the adaptor protein in the cell, as compared to the adaptor protein without the at least one additional polypeptide;
(b) the at least one additional polypeptide is disposed at an intracellular portion of the chimeric polypeptide;
(c) the at least one additional polypeptide is flanked by the adaptor protein and one of the plurality of heterologous NES domains;
(d) one of the plurality of heterologous NES domains is flanked by the adaptor protein and the at least one additional polypeptide; or
(e) the cellular component comprises a nucleic acid, a polynucleotide, an amino acid, a polypeptide, lipid, a carbohydrate, a small molecule, an enzyme, a ribosome, a proteasome, a variant thereof, or any combination thereof.

183. The chimeric polypeptide of claim 166, wherein the chimeric polypeptide further comprises a recognition moiety that is specifically recognized by an antibody.

184. The chimeric polypeptide of claim 183, wherein:
(a) upon introduction of the chimeric polypeptide into a cell, contacting of the recognition moiety by the antibody promotes or enhances (i) antibody-dependent cellular cytotoxicity, or (ii) complement-dependent cytotoxicity of the cell;
(b) the recognition moiety comprises epidermal growth factor receptor (EGFR) or a fragment thereof; or
(c) the antibody comprises at least one toxin capable of inducing death of the cell.

185. A cell comprising the chimeric polypeptide of claim 166.


SEQUENCE LISTING (10 sequences)

SEQ ID NO: 1 - protein, 258 aa, synthetic construct
  REGION 1..258: note = Description of Artificial Sequence: Synthetic polypeptide
MGLALKLAGLDIEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELNSGRLQLPPLERLTL

SEQ ID NO: 2 - protein, 10 aa, synthetic construct
  REGION 1..10: note = Description of Artificial Sequence: Synthetic peptide
LALKLAGLDI

SEQ ID NO: 3 - protein, 11 aa, synthetic construct
  REGION 1..11: note = Description of Artificial Sequence: Synthetic peptide
LQLPPLERLTL

SEQ ID NO: 4 - protein, 262 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEGASGIRGAQAGWGVWGPSWTRLTPVSLPPEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 5 - protein, 233 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 6 - protein, 232 aa, Homo sapiens
MEEAILVPCVLGLLLLPILAMLMALCVHCHRLPGSYDSTSSDSLYPRGIQFKRPHTVAPWPPAYPPVTSYPPLSQPDLLPIPSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 7 - protein, 269 aa, Homo sapiens
MEATAASWQVAVPVLGGASRPLGPRGAASLLRAPLQMEEAILVPCVLGLLLLPILAMLMALCVHCHRLPGSYDSTSSDSLYPRGIQFKRPHTVAPWPPAYPPVTSYPPLSQPDLLPIPRSPQPLGGSHRTPSSRRDSDGANSVASYENEEPACEDADEDEDDYHNPGYLVVLPDSTPATSTAAPSAPALSTPGIRDSAFSMESIDDYVNVPESGESAEASLDGSREYVNVSQELHPGAAKTEPAALSSQEAEEVEEEGAPDYENLQELN

SEQ ID NO: 8 - DNA, 30 bp, synthetic construct
  misc_feature 1..30: note = Description of Artificial Sequence: Synthetic oligonucleotide
  source 1..30: mol_type = other DNA
ctggccctgaagctggccggcctggacatc

SEQ ID NO: 9 - DNA, 33 bp, synthetic construct
  misc_feature 1..33: note = Description of Artificial Sequence: Synthetic oligonucleotide
  source 1..33: mol_type = other DNA
ctgcagctgcctccactggagagactgaccctg

SEQ ID NO: 10 - DNA, 774 bp, synthetic construct
  misc_feature 1..774: note = Description of Artificial Sequence: Synthetic polynucleotide
  source 1..774: mol_type = other DNA
atgggcctggccctgaagctggccggcctggacatcgaggaggccatcctggtcccctgcgtgctggggctcctgctgctgcccatcctggccatgttgatggcactgtgtgtgcactgccacagactgccaggctcctacgacagcacatcctcagatagtttgtatccaaggggcatccagttcaaacggcctcacacggttgccccctggccacctgcctacccacctgtcacctcctacccacccctgagccagccagacctgctccccatcccaagatccccgcagccccttgggggctcccaccggacgccatcttcccggcgggattctgatggtgccaacagtgtggcgagctacgagaacgaggaaccagcctgtgaggatgcggatgaggatgaggacgactatcacaacccaggctacctggtggtgcttcctgacagcaccccggccactagcactgctgccccatcagctcctgcactcagcacccctggcatccgagacagtgccttctccatggagtccattgatgattacgtgaacgttccggagagcggggagagcgcagaagcgtctctggatggcagccgggagtatgtgaatgtgtcccaggaactgcatcctggagcggctaagactgagcctgccgccctgagttcccaggaggcagaggaagtggaggaagagggggctccagattacgagaatctgcaggagctgaacagcggccgcctgcagctgcctccactggagagactgaccctg